(19) United States
(12) Patent Application Publication
(10) Pub. No.: US 2021/0363242 A1
(43) Pub. Date: Nov. 25, 2021

(54) METHODS OF TREATING CANCER WITH ANTIBODIES AGAINST TIM3

(71) Applicant: Bristol-Myers Squibb Company, Princeton, NJ (US)

(72) Inventors: Xiao Min SCHEBYE, San Carlos, CA (US); Mark J. SELBY, San Francisco, CA (US); Michelle Minhua HAN, Piedmont, CA (US); Christine BEE, San Francisco, CA (US); Andy X. DENG, San Mateo, CA (US); Anan CHUNTHARAPAI, Daly City, CA (US); Brigitte DEVAUX, Palo Alto, CA (US); Huiming LI, Lexington, MA (US); Paul O. SHEPPARD, Granite Falls, WA (US); Alan J. KORMAN, Piedmont, CA (US); Daniel F. ARDOUREL, Woodinville, WA (US); Ekaterina DEYANOVA, Lawrenceville, NJ (US); Richard Yu-Cheng HUANG, Bridgewater, NJ (US); Guodong CHEN, East Brunswick, NJ (US); Michelle Renne KUHNE, San Francisco, CA (US); Hong-An TRUONG, San Francisco, CA (US); Poliana PATAH, New Brunswick, NJ (US); Jeffrey R. JACKSON, Schwenksville, PA (US); Ronald A. FLEMING, Lawrenceville, NJ (US)

(73) Assignee: Bristol-Myers Squibb Company, Princeton, NJ (US)

(21) Appl. No.: 16/962,805

(22) PCT Filed: Jan. 15, 2019
(86) PCT No.: PCT/US2019/013638

§ 371 Date: Jul. 16, 2020

Related U.S. Application Data
(60) Provisional application No. 62/617,828, filed on Jan. 16, 2018.
(60) Provisional application No. 62/618,561, filed on Jan. 17, 2018.
(60) Provisional application No. 62/633,477, filed on Feb. 21, 2018.

(51) Int. Cl. C07K 16/28; A61K 39/395; A61P 35/00

(52) U.S. Cl. CPC C07K 16/2803; C07K 16/2818; A61K 2039/545; A61P 35/00; A61K 39/3955

(57) ABSTRACT

This disclosure provides a method for treating a subject afflicted with a tumor or a cancer, wherein the method comprises administering to the subject therapeutically effective amounts of an anti-TIM3 antibody, alone or in combination with an inhibitor of the PD-1 signaling pathway (e.g., anti-PD-1 antibody). In some embodiments, the antibody is administered as a flat dose or a weight-based dose.

### REFERENCE TO SEQUENCE LISTING SUBMITTED ELECTRONICALLY VIA EFS-WEB

The content of the electronically submitted sequence listing in ASCII text file (Name: 3338_117PC03_SequenceListing_ST25.txt; Size: 913,417 bytes; and Date of Creation: Jan. 14, 2019) filed with the application is herein incorporated by reference in its entirety.

### SUMMARY OF THE DISCLOSURE

Provided herein are methods of treating a tumor or a subject afflicted with a tumor or a cancer comprising administering to the subject a therapeutically effective amount of an antibody that binds specifically to a human T-cell immunoglobulin and mucin-domain containing-3 (TIM3) and, e.g., inhibits TIM3 activity (“anti-TIM3 antibody”), wherein the anti-TIM3 antibody is administered at a flat dose ranging from about 4 mg to about 960 mg or a weight-based dose ranging from about 0.05 mg/kg to about 12 mg/kg.

In some embodiments, the anti-TIM3 antibody is administered at a flat dose ranging from about 8 mg to about 800 mg, about 24 mg to about 800 mg, about 72 mg to about 800 mg, about 200 mg to about 800 mg, about 240 mg to about 800 mg, about 300 mg to about 800 mg, about 360 mg to about 800 mg, about 400 mg to about 800 mg, about 480 mg to about 800 mg, 8 mg to about 640 mg, about 24 mg to about 640 mg, about 72 mg to about 640 mg, about 200 mg to about 640 mg, about 240 mg to about 640 mg, about 300 mg to about 640 mg, about 360 mg to about 640 mg, about 400 mg to about 640 mg, about 480 mg to about 640 mg, 8 mg to about 500 mg, about 24 mg to about 500 mg, about 72 mg to about 500 mg, about 200 mg to about 500 mg, about 240 mg to about 500 mg, about 300 mg to about 500 mg, about 360 mg to about 500 mg, about 400 mg to about 500 mg, about 480 mg to about 500 mg, about 240 mg to about 480 mg, or about 360 mg to about 480 mg. In some embodiments, the anti-TIM3 antibody is administered at a flat dose of about 8 mg, about 24 mg, about 50 mg, about 72 mg, about 100 mg, about 150 mg, about 200 mg, about 240 mg, about 250 mg, about 300 mg, about 350 mg, about 360 mg, about 400 mg, about 450 mg, about 480 mg, about 500 mg, about 540 mg, about 560 mg, about 600 mg, about 640 mg, about 650 mg, about 660 mg, about 700 mg, about 720 mg, about 750 mg, about 760 mg, or about 800 mg.

In some embodiments, the anti-TIM3 antibody is administered at a weight-based dose ranging from about 0.1 mg/kg to about 10 mg/kg, about 0.3 mg/kg to about 10 mg/kg, 0.9 mg/kg to about 10 mg/kg, about 1 mg/kg to about 10 mg/kg, about 2.5 mg/kg to about 10 mg/kg, about 3 mg/kg to about 10 mg/kg, about 4 mg/kg to about 10 mg/kg, about 5 mg/kg to about 10 mg/kg, about 6 mg/kg to about 10 mg/kg, about 7 mg/kg to about 10 mg/kg, about 8 mg/kg to about 10 mg/kg, about 9 mg/kg to about 10 mg/kg, about 0.1 mg/kg to about 8 mg/kg, about 0.3 mg/kg to about 8 mg/kg, 0.9 mg/kg to about 8 mg/kg, about 1 mg/kg to about 8 mg/kg, about 2.5 mg/kg to about 8 mg/kg, about 3 mg/kg to about 8 mg/kg, about 4 mg/kg to about 8 mg/kg, about 5 mg/kg to about 8 mg/kg, about 6 mg/kg to about 8 mg/kg, about 7 mg/kg to about 8 mg/kg, about 0.1 mg/kg to about 6 mg/kg, about 0.3 mg/kg to about 6 mg/kg, 0.9 mg/kg to about 6 mg/kg, about 1 mg/kg to about 6 mg/kg, about 2.5 mg/kg to about 6 mg/kg, about 3 mg/kg to about 6 mg/kg, about 4 mg/kg to about 6 mg/kg, or about 5 mg/kg to about 6 mg/kg. In some embodiments, the anti-TIM3 antibody is administered at a weight-based dose of about 0.1 mg/kg, about 0.3 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, or about 12 mg/kg.

In some embodiments, the methods of the present disclosure further comprise administering a therapeutically effective amount of an anti-PD-1 antibody. In some embodiments, the anti-PD-1 antibody is administered at a flat dose ranging from about 80 mg to about 640 mg or a weight-based dose ranging from about 1 mg/kg to about 8 mg/kg.

In some embodiments, the anti-PD-1 antibody is administered at a flat dose ranging from about 100 mg to about 640 mg, about 120 mg to about 640 mg, about 150 mg to about 640 mg, about 160 mg to about 640 mg, about 180 mg to about 640 mg, about 240 mg to about 640 mg, about 300 mg to about 640 mg, about 320 mg to about 640 mg, about 360 mg to about 640 mg, about 400 mg to about 640 mg, about 420 mg to about 640 mg, about 480 mg to about 640 mg, about 540 mg to about 640 mg, about 100 mg to about 540 mg, about 120 mg to about 540 mg, about 150 mg to about 540 mg, about 160 mg to about 540 mg, about 180 mg to about 540 mg, about 240 mg to about 540 mg, about 300 mg to about 540 mg, about 320 mg to about 540 mg, about 360 mg to about 540 mg, about 400 mg to about 540 mg, about 420 mg to about 540 mg, about 480 mg to about 540 mg, about 100 mg to about 480 mg, about 120 mg to about 480 mg, about 150 mg to about 480 mg, about 160 mg to about 480 mg, about 180 mg to about 480 mg, about 240 mg to about 480 mg, about 300 mg to about 480 mg, about 320 mg to about 480 mg, about 360 mg to about 480 mg, about 400 mg to about 480 mg, about 420 mg to about 480 mg, about 240 mg to about 400 mg, about 300 mg to about 400 mg, about 320 mg to about 400 mg, or about 360 mg to about 400 mg. In some embodiments, the anti-PD-1 antibody is administered at a flat dose of about 160 mg, about 200 mg, about 240 mg, about 300 mg, about 360 mg, about 420 mg, about 450 mg, about 480 mg, about 500 mg, about 540 mg, about 600 mg, or about 640 mg.

In some embodiments, the anti-PD-1 antibody is administered at a weight-based dose ranging from about 1 mg/kg to about 7 mg/kg, about 1 mg/kg to about 6 mg/kg, about 1 mg/kg to about 5 mg/kg, about 1 mg/kg to about 4 mg/kg, about 1 mg/kg to about 3 mg/kg, about 1 mg/kg to about 2 mg/kg, about 2 mg/kg to about 7 mg/kg, about 2 mg/kg to about 6 mg/kg, about 2 mg/kg to about 5 mg/kg, about 2 mg/kg to about 4 mg/kg, about 2 mg/kg to about 3 mg/kg, about 3 mg/kg to about 7 mg/kg, about 3 mg/kg to about 6 mg/kg, about 3 mg/kg to about 5 mg/kg, about 3 mg/kg to about 4 mg/kg, about 4 mg/kg to about 7 mg/kg, about 4 mg/kg to about 6 mg/kg, about 4 mg/kg to about 5 mg/kg, about 5 mg/kg to about 7 mg/kg, about 5 mg/kg to about 6 mg/kg, or about 6 mg/kg to about 7 mg/kg. In some embodiments, the anti-PD-1 antibody is administered at a weight-based dose of about 1 mg/kg, about 2 mg/kg, about 3 mg/kg, about 4 mg/kg, about 5 mg/kg, about 6 mg/kg, about 7 mg/kg, or about 8 mg/kg.

In some embodiments, the anti-TIM3 antibody is administered at a dosing interval of about 1, 2, 3, 4, 5, or 6 weeks. In some embodiments, the anti-PD-1 antibody is administered at a dosing interval of about 1, 2, 3, 4, 5, or 6 weeks.

In some embodiments, the anti-TIM3 antibody is administered at a flat dose of about 200 mg and the anti-PD-1 antibody is administered at a flat dose of about 480 mg. In some embodiments, the anti-TIM3 antibody is administered at a flat dose of about 480 mg and the anti-PD-1 antibody is administered at a flat dose of about 480 mg. In some embodiments, the anti-TIM3 antibody is administered at a flat dose of about 800 mg and the anti-PD-1 antibody is administered at a flat dose of about 480 mg. In some embodiments, the anti-TIM3 antibody is administered at a dosing interval of 4 weeks. In some embodiments, the anti-PD-1 antibody is administered at a dosing interval of 4 weeks.

In some embodiments, the anti-TIM3 antibody is administered at a flat dose of about 4 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 8 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 72 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 150 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 480 mg at a dosing interval of 4 weeks and the anti-PD-1 antibody is administered at a flat dose of about 480 mg at a dosing interval of 4 weeks.

In some embodiments, the anti-TIM3 antibody is administered to the subject prior to the administration of the anti-PD-1 antibody. In some embodiments, the anti-TIM3 antibody is administered to the subject after the administration of the anti-PD-1 antibody. In some embodiments, the anti-TIM3 antibody and the anti-PD-1 antibody are administered concurrently in separate compositions. In some embodiments, the anti-TIM3 antibody and the anti-PD-1 antibody are admixed as a single composition for concurrent administration.

In some embodiments, the tumor is derived from a cancer selected from the group consisting of a bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, renal cancer, lung cancer, stomach cancer, germ cell cancer, bone cancer, liver cancer, thyroid cancer, skin cancer, neoplasm of the central nervous system, lymphoma, leukemia, myeloma, sarcoma, virus-related cancer, and any combinations thereof. In some embodiments, the cancer is an advanced, recurring, metastatic, and/or refractory cancer. In some embodiments, the cancer is a renal cancer (e.g., renal cell carcinoma). In some embodiments, the cancer is a colorectal cancer (e.g., colorectal carcinoma). In some embodiments, the cancer is a lung cancer (e.g., non-small cell lung cancer). In some embodiments, the cancer is a head and neck cancer (e.g., squamous carcinoma of the head and neck). In some embodiments, the cancer is a breast cancer (e.g., triple negative breast cancer). In some embodiments, the cancer is a skin cancer (e.g., melanoma). In some embodiments, the cancer is a bladder cancer (e.g., urothelial carcinoma). In some embodiments, the cancer is a lymphoma (e.g., classical Hodgkin's lymphoma). In some embodiments, the cancer is a liver cancer (e.g., hepatocellular carcinoma).

In some embodiments, the cancer is refractory to a prior cancer therapy selected from the group consisting of an anti-angiogenic therapy regimen (e.g., sunitinib, sorafenib, pazopanib, axitinib, tivozanib, and bevacizumab), a standard systemic therapy for metastatic and/or unresectable disease (e.g., Oxaliplatin and Irinotecan), platinum-based chemotherapy, anti-PD(L)-1 therapy, and any combinations thereof.

In some embodiments, the tumor comprises one or more cells that express human TIM3. In some embodiments, the tumor comprises one or more cells that express PD-L1, PD-L2, or both.

In some embodiments, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some embodiments, the administration reduces the size of the tumor relative to the size of the tumor prior to the administration. In some embodiments, the size of the tumor is reduced by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to the size of the tumor prior to the administration.

In some embodiments, the administration induces a proliferation of tumor infiltrating lymphocytes (TILs) in the tumor.

In some embodiments, the anti-PD-1 antibody cross-competes with nivolumab. In some embodiments, the anti-PD-1 antibody is nivolumab.

In some embodiments, the anti-TIM3 antibody cross-competes for binding to human TIM3 with a reference antibody selected from Table 2. In some embodiments, the anti-TIM3 antibody binds to human TIM3 at a same epitope as the reference antibody, as determined by HDX.

In some embodiments, the anti-TIM3 antibody comprises a heavy chain CDR1, CDR2, and CDR3 and a light chain CDR1, CDR2, and CDR3, wherein

(i) the heavy chain CDR1 comprises an amino acid sequence selected from the group consisting of SEQ ID NO: 41, SEQ ID NO: 42, SEQ ID NO: 43, SEQ ID NO: 44, and SEQ ID NO: 45;

(ii) the heavy chain CDR2 comprises an amino acid sequence selected from the group consisting of SEQ ID NO: 46, SEQ ID NO: 122, SEQ ID NO: 123, SEQ ID NO: 124, SEQ ID NO: 47, SEQ ID NO: 125, SEQ ID NO: 48, SEQ ID NO: 49, SEQ ID NO: 50, SEQ ID NO: 51, SEQ ID NO: 52, SEQ ID NO: 413, and SEQ ID NO: 415;

(iii) the heavy chain CDR3 comprises an amino acid sequence selected from the group consisting of SEQ ID NO: 53, SEQ ID NO: 126, SEQ ID NO: 127, SEQ ID NO: 128, SEQ ID NO: 129, SEQ ID NO: 128, SEQ ID NO: 54, SEQ ID NO: 55, SEQ ID NO: 56, SEQ ID NO: 57, SEQ ID NO: 58, SEQ ID NO: 59, SEQ ID NO: 414, and SEQ ID NO: 416;

(iv) the light chain CDR1 comprises an amino acid sequence selected from the group consisting of SEQ ID NO: 64 and SEQ ID NO: 65;

(v) the light chain CDR2 comprises an amino acid sequence selected from the group consisting of SEQ ID NO: 66 and SEQ ID NO: 67; and/or

(vi) the light chain CDR3 comprises an amino acid sequence selected from the group consisting of SEQ ID NO: 68, SEQ ID NO: 69, SEQ ID NO: 70, SEQ ID NO: 71; and SEQ ID NO: 419.

In some embodiments, the anti-TIM3 antibody comprises a heavy chain CDR1, CDR2, and CDR3 and a light chain CDR1, CDR2, and CDR3, wherein

(a1) the heavy chain CDR1, CDR2, and CDR3 comprise the amino acid sequences of SEQ ID NOs: 41, 46, and 53, respectively, and the light chain CDR1, CDR2, and CDR3 comprise the amino acid sequences of SEQ ID NOs: 64, 66, and 68, respectively;

(a2) the heavy chain CDR1, CDR2, and CDR3 comprise the amino acid sequences of SEQ ID NOs: 41, 122, and 53, respectively, and the light chain CDR1, CDR2, and CDR3 comprise the amino acid sequences of SEQ ID NOs: 64, 66, and 68, respectively;

(a3) the heavy chain CDR1, CDR2, and CDR3 comprise the amino acid sequences of SEQ ID NOs: 41, 123, and 53, respectively, and the light chain CDR1, CDR2, and CDR3 comprise the amino acid sequences of SEQ ID NOs: 64, 66, and 68, respectively;

(a4) the heavy chain CDR1, CDR2, and CDR3 comprise the amino acid sequences of SEQ ID NOs: 41, 124, and 53, respectively, and the light chain CDR1, CDR2, and CDR3 comprise the amino acid sequences of SEQ ID NOs: 64, 66, and 68, respectively;

(a5) the heavy chain CDR1, CDR2, and CDR3 comprise the amino acid sequences of SEQ ID NOs: 41, 46, and 126, respectively, and the light chain CDR1, CDR2, and CDR3 comprise the amino acid sequences of SEQ ID NOs: 64, 66, and 68, respectively;

(a6) the heavy chain CDR1, CDR2, and CDR3 comprise the amino acid sequences of SEQ ID NOs: 41, 46, and 127, respectively, and the light chain CDR1, CDR2, and CDR3 comprise the amino acid sequences of SEQ ID NOs: 64, 66, and 68, respectively;

(a7) the heavy chain CDR1, CDR2, and CDR3 comprise the amino acid sequences of SEQ ID NOs: 41, 46, and 128, respectively, and the light chain CDR1, CDR2, and CDR3 comprise the amino acid sequences of SEQ ID NOs: 64, 66, and 68, respectively;

(a8) the heavy chain CDR1, CDR2, and CDR3 comprise the amino acid sequences of SEQ ID NOs: 41, 46, and 129, respectively, and the light chain CDR1, CDR2, and CDR3 comprise the amino acid sequences of SEQ ID NOs: 64, 66, and 68, respectively;

(a9) the heavy chain CDR1, CDR2, and CDR3 comprise the amino acid sequences of SEQ ID NOs: 41, 122, and 128, respectively, and the light chain CDR1, CDR2, and CDR3 comprise the amino acid sequences of SEQ ID NOs: 64, 66, and 68, respectively;

(a10) the heavy chain CDR1, CDR2, and CDR3 comprise the amino acid sequences of SEQ ID NOs: 41, 122, and 126, respectively, and the light chain CDR1, CDR2, and CDR3 comprise the amino acid sequences of SEQ ID NOs: 64, 66, and 68, respectively;

(b1) the heavy chain CDR1, CDR2, and CDR3 comprise the amino acid sequences of SEQ ID NOs: 42, 47, and 54, respectively, and the light chain CDR1, CDR2, and CDR3 comprise the amino acid sequences of SEQ ID NOs: 64, 66, and 69, respectively;

(b2) the heavy chain CDR1, CDR2, and CDR3 comprise the amino acid sequences of SEQ ID NOs: 42, 125, and 54, respectively, and the light chain CDR1, CDR2, and CDR3 comprise the amino acid sequences of SEQ ID NOs: 64, 66, and 69, respectively;

(c) the heavy chain CDR1, CDR2, and CDR3 comprises the amino acid sequences of SEQ ID NOs: 43, 48, and 55, respectively, and the light chain CDR1, CDR2, and CDR3 comprises the amino acid sequences of SEQ ID NOs: 64, 66, and 69, respectively;

(d) the heavy chain CDR1, CDR2, and CDR3 comprise the amino acid sequences of SEQ ID NOs: 44, 49, and 56, respectively, and the light chain CDR1, CDR2, and CDR3 comprise the amino acid sequences of SEQ ID NOs: 64, 66, and 68, respectively;

(e1) the heavy chain CDR1, CDR2, and CDR3 comprise the amino acid sequences of SEQ ID NOs: 45, 50, and 57, respectively, and the light chain CDR1, CDR2, and CDR3 comprise the amino acid sequences of SEQ ID NOs: 64, 66, and 69, respectively;

(e2) the heavy chain CDR1, CDR2, and CDR3 comprise the amino acid sequences of SEQ ID NOs: 45, 50, and 57, respectively, and the light chain CDR1, CDR2, and CDR3 comprise the amino acid sequences of SEQ ID NOs: 64, 66, and 71, respectively;

(e3) the heavy chain CDR1, CDR2, and CDR3 comprise the amino acid sequences of SEQ ID NOs: 45, 50, and 57, respectively, and the light chain CDR1, CDR2, and CDR3 comprise the amino acid sequences of SEQ ID NOs: 65, 67, and 70, respectively;

(f) the heavy chain CDR1, CDR2, and CDR3 comprise the amino acid sequences of SEQ ID NOs: 45, 51, and 58, respectively, and the light chain CDR1, CDR2, and CDR3 comprise the amino acid sequences of SEQ ID NOs: 64, 66, and 68, respectively;

(g) the heavy chain CDR1, CDR2, and CDR3 comprise the amino acid sequences of SEQ ID NOs: 45, 52, and 59, respectively, and the light chain CDR1, CDR2, and CDR3 comprise the amino acid sequences of SEQ ID NOs: 64, 66, and 69, respectively;

(h) the heavy chain CDR1, CDR2, and CDR3 comprise the amino acid sequences of SEQ ID NOs: 45, 413, and 414, respectively, and the light chain CDR1, CDR2, and CDR3 comprise the amino acid sequences of SEQ ID NOs: 64, 66, and 69, respectively;

(i1) the heavy chain CDR1, CDR2, and CDR3 comprise the amino acid sequences of SEQ ID NOs: 45, 415, and 416, respectively, and the light chain CDR1, CDR2, and CDR3 comprise the amino acid sequences of SEQ ID NOs: 64, 66, and 68, respectively; or

(i2) the heavy chain CDR1, CDR2, and CDR3 comprise the amino acid sequences of SEQ ID NOs: 45, 415, and 416, respectively, and the light chain CDR1, CDR2, and CDR3 comprise the amino acid sequences of SEQ ID NOs: 64, 66, and 419, respectively.

In some embodiments, the anti-TIM3 antibody comprises:

(1) a heavy chain variable region comprising an amino acid sequence selected from the group consisting of SEQ ID NO: 34, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 364, SEQ ID NO: 35, SEQ ID NO: 120, SEQ ID NO: 36, SEQ ID NO: 37, SEQ ID NO: 38, SEQ ID NO: 121; SEQ ID NO: 39, SEQ ID NO: 40, SEQ ID NO: 410, SEQ ID NO: 411, and SEQ ID NO: 412; and/or

(2) a light chain variable region comprising an amino acid sequence selected from the group consisting of SEQ ID NO: 60, SEQ ID NO: 61, SEQ ID NO: 62, SEQ ID NO: 63; SEQ ID NO: 417, and SEQ ID NO: 418.

In some embodiments, the anti-TIM3 antibody is selected from the group consisting an IgG1, an IgG2, an IgG3, an IgG4, and a variant thereof. In some embodiments, the anti-TIM3 antibody is an IgG1 antibody. In some embodiments, the anti-TIM3 antibody comprises an effectorless IgG1 Fc.

In some embodiments, the anti-TIM3 antibody comprises:

(1) a heavy chain comprising an amino acid sequence selected from the group consisting of SEQ ID NO: 15 (or 22), SEQ ID NO: 92 (or 102), SEQ ID NO: 93 (or 103), SEQ ID NO: 94 (or 104), SEQ ID NO: 95 (or 105), SEQ ID NO: 96 (or 106), SEQ ID NO: 97 (or 107), SEQ ID NO: 98 (or 108), SEQ ID NO: 99 or (109), SEQ ID NO: 351 (or 352), SEQ ID NO: 16 (or 23), SEQ ID NO: 100 or (110), SEQ ID NO: 17 (or 24), SEQ ID NO: 18 (or 25), SEQ ID NO: 19 (or 26), SEQ ID NO: 101 (or 111), SEQ ID NO: 20 (or 27), SEQ ID NO: 21 (or 28), SEQ ID NO: 390 (or 391), SEQ ID NO: 398 (or 399), and SEQ ID NO: 404 (or 405); and/or

(2) a light chain comprising an amino acid sequence selected from the group consisting of SEQ ID NO: 29, SEQ ID NO: 30, SEQ ID NO: 33, and SEQ ID NO: 408.

In some embodiments, the anti-TIM3 antibody comprises a heavy chain and a light chain, wherein:

(a1) the heavy chain comprises the amino acid sequence of SEQ ID NO: 15 (or 22) and the light chain comprises the amino acid sequence of SEQ ID NO: 29;

(a2) the heavy chain comprises the amino acid sequence of SEQ ID NO: 92 (or 102) and the light chain comprises the amino acid sequence of SEQ ID NO: 29;

(a3) the heavy chain comprises the amino acid sequence of SEQ ID NO: 93 (or 103) and the light chain comprises the amino acid sequence of SEQ ID NO: 29;

(a4) the heavy chain comprises the amino acid sequence of SEQ ID NO: 94 (or 104) and the light chain comprises the amino acid sequence of SEQ ID NO: 29;

(a5) the heavy chain comprises the amino acid sequence of SEQ ID NO: 95 (or 105) and the light chain comprises the amino acid sequence of SEQ ID NO: 29;

(a6) the heavy chain comprises the amino acid sequence of SEQ ID NO: 96 (or 106) and the light chain comprises the amino acid sequence of SEQ ID NO: 29;

(a7) the heavy chain comprises the amino acid sequence of SEQ ID NO: 97 (or 107) and the light chain comprises the amino acid sequence of SEQ ID NO: 29;

(a8) the heavy chain comprises the amino acid sequence of SEQ ID NO: 98 (or 108) and the light chain comprises the amino acid sequence of SEQ ID NO: 29;

(a9) the heavy chain comprises the amino acid sequence of SEQ ID NO: 99 or (109) and the light chain comprises the amino acid sequence of SEQ ID NO: 29;

(a10) the heavy chain comprises the amino acid sequence of SEQ ID NO: 351 (or 352) and the light chain comprises the amino acid sequence of SEQ ID NO: 29;

(b1) the heavy chain comprises the amino acid sequence of SEQ ID NO: 16 (or 23) and the light chain comprises the amino acid sequence of SEQ ID NO: 30;

(b2) the heavy chain comprises the amino acid sequence of SEQ ID NO: 100 or (110) and the light chain comprises the amino acid sequence of SEQ ID NO: 30;

(c) the heavy chain comprises the amino acid sequence of SEQ ID NO: 17 (or 24) and the light chain comprises the amino acid sequence of SEQ ID NO: 30;

(d) the heavy chain comprises the amino acid sequence of SEQ ID NO: 18 (or 25) and the light chain comprises the amino acid sequence of SEQ ID NO: 29;

(e1) the heavy chain comprises the amino acid sequence of SEQ ID NO: 19 (or 26) and the light chain comprises the amino acid sequence of SEQ ID NO: 33;

(e2) the heavy chain comprises the amino acid sequence of SEQ ID NO: 101 (or 111) and the light chain comprises the amino acid sequence of SEQ ID NO: 33;

(f) the heavy chain comprises the amino acid sequence of SEQ ID NO: 20 (or 27) and the light chain comprises the amino acid sequence of SEQ ID NO: 29;

(g) the heavy chain comprises the amino acid sequence of SEQ ID NO: 21 (or 28) and the light chain comprises the amino acid sequence of SEQ ID NO: 30;

(h) the heavy chain comprises the amino acid sequence of SEQ ID NO: 390 (or 391) and the light chain comprises the amino acid sequence of SEQ ID NO: 408;

(i1) the heavy chain comprises the amino acid sequence of SEQ ID NO: 398 (or 399) and the light chain comprises the amino acid sequence of SEQ ID NO: 29; or

(i2) the heavy chain comprises the amino acid sequence of SEQ ID NO: 404 (or 405) and the light chain comprises the amino acid sequence of SEQ ID NO: 29.

In some embodiments, the administration is performed in combination with an additional therapeutic agent. In some embodiments, the additional therapeutic agent is selected from the group consisting of a chemotherapy, radiation, surgery, hormone deprivation, angiogenesis inhibitors, additional immune checkpoint inhibitors, and any combinations thereof. In some embodiments, the additional immune checkpoint inhibitors comprise an anti-LAG-3 antibody, an anti-CTLA-4 antibody, an anti-GITR antibody, or an anti-PD-L1 antibody.

### DETAILED DESCRIPTION OF DISCLOSURE

This disclosure relates to methods for treating a tumor or a subject afflicted with a tumor or a cancer comprising administering to the subject an anti-TIM3 antibody that inhibits TIM3 activity. In some embodiments, the anti-TIM3 antibody is administered at a flat dose or a weight-based dose. In some embodiments, the anti-TIM3 antibody is administered in combination with another therapeutic agent (e.g., an inhibitor of the PD-1 signaling pathway, e.g., an anti-PD-1 antibody). In some embodiments, the tumor is a solid tumor, e.g., an advanced and/or metastatic solid tumor.

### Definitions

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “overdose” refers to the accidental or intentional administration of any dose of a product that is considered both excessive and medically important.

As used herein, “dose limiting toxicities” are defined based on the incidence, intensity, and duration of an AE for which no clear alternative cause is identified. Participants experiencing a DLT will not be retreated with study drug, and will enter the safety follow-up period of the study.

In some embodiments, any one of the following study drug-related events will be considered a hepatic DLT: (1) Grade 4 elevations in serum transaminases (AST, ALT), alkaline phosphatase (ALP) or total bilirubin, in the absence of cholestasis; (2) Grade 3 elevations in serum transaminases (AST, ALT) or alkaline phosphatase (ALP) in the absence of cholestasis, lasting longer than 5 days; (3) Grade 2 elevations in AST or ALT with symptomatic liver inflammation (e.g., right upper quadrant tenderness, jaundice, and pruritus); or (4) AST or ALT>3×ULN and concurrent total bilirubin>2×ULN without initial findings of cholestasis (elevated ALP, e.g., findings consistent with Hy's law or FDA definition of potential drug-induced liver injury [pDILI]).

In some embodiments, any one of the following study drug-related events will be considered a hematologic DLT: (1) Grade 4 neutropenia≥7 days in duration; (2) Grade 4 thrombocytopenia; (3) Grade 3 thrombocytopenia with bleeding, or any requirement for platelet transfusion; (4) Febrile neutropenia; (5) Grade 3 hemolysis (i.e., requiring transfusion or medical intervention such as steroids); or (6) Grade 4 anemia not explained by underlying disease.

In some embodiments, any one of the following study drug-related events will be considered a dermatologic DLT: (1) Grade 4 rash; (2) Grade 3 rash if no improvement (i.e., resolution to <Grade 1) after a 1 to 2 week infusion delay.

In some embodiments, any of the following events will be considered a DLT: (1) Grade 2 drug-related uveitis, episcleritis, iritis eye pain or blurred vision that does not respond to topical therapy and does not improve to Grade 1 severity until the following dose OR requires systemic treatment; (2) Grade 3 drug-related uveitis, episcleritis, iritis, pneumonitis, bronchospasm or neurologic toxicity; (3) Grade 4 hypersensitivity reaction, or Grade 3 that does not resolve to Grade 1 in <6 hours; (4) Grade ≥2 colitis that lasts more than 5 days; (5) Grade ≥3 colitis that lasts more than 48 hours; or (6) Grade ≥3 colitis that lasts more than 48 hours.

In some embodiments, other ≥Grade 3 study drug-related toxicity will be considered a DLT, with the exception of the following: (1) Grade 3 electrolyte abnormalities that are not complicated by associated clinical adverse experiences, last less than 72 hours and either resolve spontaneously or respond to conventional medical intervention; (2) Grade 3 nausea, vomiting, or diarrhea that lasts less than 48 hours, and either resolves spontaneously or responds to conventional medical intervention; (3) Grade 3 or grade 4 elevation of amylase or lipase not associated with clinical or radiographic evidence of pancreatitis; (4) Grade 3 fever not associated with hemodynamic compromise (e.g., hypotension, clinical or laboratory evidence of impaired end-organ perfusion); (5) Grade 3 endocrinopathy that is well controlled by hormone replacement; (6) Grade 3 tumor flare (defined as pain, irritation, or rash that localizes to sites of known or suspected tumor); and (7) Grade 3 fatigue; and (8) Grade ≤3 infusion reaction that returns to Grade 1 in <6 hours.

The term “T-cell immunoglobulin and mucin-domain containing-3” or “TIM3” as used herein refers to a receptor that is a member of the T cell immunoglobulin and mucin domain (TIM) family of proteins. Primary ligand for TIM3 include phosphatidylserine (TIM3-L). TIM3 is also referred to as hepatitis A virus cellular receptor 2 (HAVCR2), T-cell immunoglobulin mucin receptor 3, TIM-3, TIMD3, TIMD-3, Kidney Injury Molecule-3, KIM-3, and CD366. The term “TIM3” includes any variants or isoforms of TIM3 which are naturally expressed by cells. Accordingly, antibodies described herein can cross-react with TIM3 from species other than human (e.g., cynomolgus TIM3). Alternatively, the antibodies can be specific for human TIM3 and do not exhibit any cross-reactivity with other species. TIM3 or any variants and isoforms thereof, can either be isolated from cells or tissues which naturally express them or be recombinantly produced using well-known techniques in the art and/or those described herein.

Two isoforms of human TIM3 have been identified. Isoform 1 (Accession No. NP_116171; SEQ ID NO: 286) consists of 301 amino acids and represents the canonical sequence. Isoform 2 (Accession No. AAH20843; SEQ ID NO: 287) consists of 142 amino acids, and is soluble. It lacks amino acid residues 143-301, which encode the transmembrane domain, the cytoplasmic domain, and part of the extracellular domain of TIM3. The amino acid residues 132-142 also differ from the canonical sequence described above.

Below are the amino acid sequences of the two known human TIM3 isoforms.

(A) Human TIM3 isoform 1 (Accession No. NP_116171; SEQ ID NO: 286; encoded by the nucleotide sequence having Accession No. NM_032782.4; SEQ ID NO: 288):

MFSHLPFDCVLLLLLLLLTRSSEVEYRAEVGQNAYLPCFYTPAAPGNLV
PVCWGKGACPVFECGNVVLRTDERDVNYWTSRYWLNGDFRKGDVSLTIE
NVTLADSGIYCCRIQIPGIMNDEKFNLKLVIKPAKVTPAPTRQRDFTAA
FPRMLTTRGHGPAETQTLGSLPDINLTQISTLANELRDSRLANDLRDSG
ATIRIGIYIGAGICAGLALALIFGALIFKWYSHSKEKIQNLSLISLANL
PPSGLANAVAEGIRSEENIYTIEENVYEVEEPNEYYCYVSSRQQPSQPL
GCRFAMP

(B) Human TIM3 isoform 2 (Accession No. AAH20843; SEQ ID NO: 287; encoded by the nucleotide sequence having Accession No. BC020843.1; SEQ ID NO: 289):

MFSHLPFDCVLLLLLLLLTRSSEVEYRAEVGQNAYLPCFYTPAAPGNLV
PVCWGKGACPVFECGNVVLRTDERDVNYWTSRYWLNGDFRKGDVSLTIE
NVTLADSGIYCCRIQIPGIMNDEKFNLKLVIKPGEWTFACHLYE

The signal sequence of isoforms 1 and 2 corresponds to amino acids 1-21 (underlined). Thus, the mature isoforms 1 and 2 consist of amino acids 22 to 301 or 142, respectively. The extracellular domain of mature human TIM3 consists of amino acids 22-202 of SEQ ID NO: 286 and has the amino acid sequence:

(SEQ ID NO: 290)
SEVEYRAEVGQNAYLPCFYIPAAPGNLVPVCWGKGACPVFECGNVVLRI
DERDVNYWTSRYWLNGDFRKGDVSLTIENVTLADSGIYCCRIQIPGIMN
DEKFNLKLVIKPAKVITAPTRQRDFTAAFPRMLITRGHGPAETQTLGSL
PDINLTQISTLANELRDSRLANDLRDSGATIRIG.

Cynomolgus TIM3 protein consists of the following amino acid sequence (including a signal sequence):

(SEQ ID NO: 360)
MFSHLPFDCVLLLLLLLLTRSSEVEYIAEVGQNAYLPCSYTPAPPGNLV
PVCWGKGACPVFDCSNVVLRTENRDVNDRTSGRYWLKGDFHKGDVSLTI
ENVTLADSGVYCCRIQIPGIMNDEKHNLKLVVIKPAKVTPAPTLQRDLT
SAFPRMLTTGEHGPAETQTPGSLPDVNLTQIFTLTNELRDSGATIRTAI
YIAAGISAGLALALIFGALIFKWYSHSKEKTQNLSLISLANIPPSGLAN
AVAEGIRSEENIYTIEEDVYEVEEPNEYYCYVSSGQQPSQPLGCRFAMP

The term “antibody” refers, in some embodiments, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In certain antibodies, e.g., naturally-occurring IgG antibodies, the heavy chain constant region is comprised of a hinge and three domains, CH1, CH2 and CH3. In certain antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. A heavy chain may have the C-terminal lysine or not. Unless specified otherwise herein, the amino acids in the variable regions are numbered using the Kabat numbering system and those in the constant regions are numbered using the EU system.

An “IgG antibody”, e.g., a human IgG1, IgG2, IgG3 and IgG4 antibody, as used herein has, in some embodiments, the structure of a naturally-occurring IgG antibody, i.e., it has the same number of heavy and light chains and disulfide bonds as a naturally-occurring IgG antibody of the same subclass. For example, an anti-TIM3 IgG1, IgG2, IgG3 or IgG4 antibody consists of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, 10⁻⁸ M or less, 5×10⁻⁹ M or less, or between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to human TIM3 can, in some embodiments, also have cross-reactivity with TIM3 antigens from certain primate species (e.g., cynomolgus TIM3), but cannot cross-react with TIM3 antigens from other species or with an antigen other than TIM3.

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some embodiments, the anti-TIM3 antibodies described herein are of the IgG1 subtype. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen (e.g., human TIM3). It has been shown that the antigen-binding function of an antibody can be performed by fragments of a full-length antibody. Examples of binding fragments encompassed within the term “antigen-binding portion” of an antibody, e.g., an anti-TIM3 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody,” as used herein, refers to an antibody from a population of substantially homogeneous antibodies, i.e., the individual antibodies comprised in the population are substantially similar and bind the same epitope(s) (e.g., the antibodies display a single binding specificity and affinity), except for possible variants that may arise during production of the monoclonal antibody, such variants generally being present in minor amounts. The modifier “monoclonal” indicates the character of the antibody as being obtained from a substantially homogeneous population of antibodies, and is not to be construed as requiring production of the antibody by any particular method. The term “human monoclonal antibody” refers to an antibody from a population of substantially homogeneous antibodies that display(s) a single binding specificity and which has variable and optional constant regions derived from human germline immunoglobulin sequences. In some embodiments, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The anti-TIM3 antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody are replaced with corresponding amino acids derived from human immunoglobulins. In some embodiments of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally-occurring variants within a specific isotype group, which variants differ in a few amino acids (see, e.g., Jefferis et al. (2009) mAbs 1:1). Anti-TIM3 antibodies described herein can be of any allotype. As used herein, antibodies referred to as “IgG1f,” “IgG1.1f,” or “IgG1.3f” isotype are IgG1, effectorless IgG1.1, and effectorless IgG1.3 antibodies, respectively, of the allotype “f,” i.e., having 214R, 356E and 358M according to the EU index as in Kabat, as shown, e.g., in SEQ ID NO: 3.

The phrases “an antibody recognizing an antigen” and “an antibody specific for an antigen” are used interchangeably herein with the term “an antibody which binds specifically to an antigen.”

An “isolated antibody” refers to an antibody that is substantially free of other antibodies having different antigenic specificities (e.g., an isolated antibody that binds specifically to TIM3 is substantially free of antibodies that bind specifically to antigens other than TIM3). An isolated antibody that binds specifically to TIM3 can, however, have cross-reactivity to other antigens, such as TIM3 molecules from different species. Moreover, an isolated antibody can be substantially free of other proteins and cellular material. In some embodiments, an antibody includes a conjugate attached to another agent (e.g., small molecule drug).

As used herein, an antibody that “inhibits binding of TIM3-L to TIM3” is intended to refer to an antibody that inhibits the binding of TIM3 to its ligand, e.g., phosphatidylserine, e.g., in binding assays using CHO cells transfected with human TIM3 or TIM3 expressing activated T cells, with an EC₅₀ of about 1 μg/mL or less, such as about 0.9 μg/mL or less, about 0.85 μg/mL or less, about 0.8 μg/mL or less, about 0.75 μg/mL or less, about 0.7 μg/mL or less, about 0.65 μg/mL or less, about 0.6 μg/mL or less, about 0.55 μg/mL or less, about 0.5 μg/mL or less, about 0.45 μg/mL or less, about 0.4 μg/mL or less, about 0.35 μg/mL or less, about 0.3 μg/mL or less, about 0.25 μg/mL or less, about 0.2 μg/mL or less, about 0.15 μg/mL or less, about 0.1 μg/mL or less, or about 0.05 μg/mL or less, in art-recognized methods, e.g., the FACS-based binding assays described herein.

An “effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally-occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc include the various allotypes of Fes (see, e.g., Jefferis et al. (2009) mAbs 1: 1).

The term “epitope” or “antigenic determinant” refers to a site on an antigen (e.g., TIM3) to which an immunoglobulin or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from TIM3) are tested for reactivity with a given antibody (e.g., anti-TIM3 antibody). Methods of determining spatial conformation of epitopes include techniques in the art and those described herein, for example, x-ray crystallography, antigen mutational analysis, 2-dimensional nuclear magnetic resonance and HDX-MS (see, e.g., Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on TIM3” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some embodiments, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, N.Y., USA 1999. Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include: competition for binding to T cells expressing TIM3, e.g., by flow cytometry, such as described in the Examples. Other methods include: SPR (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., recombinant human TIM3, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human TIM3” refers to an antibody that binds to soluble or cell bound human TIM3 with a K_{D} of 10⁻⁷ M or less, such as approximately less than 10⁻⁸ M, 10⁻⁹ M or 10⁻¹⁰ M or even lower. An antibody that “cross-reacts with cynomolgus TIM3” refers to an antibody that binds to cynomolgus TIM3 with a K_{D} of 10⁻⁷ M or less, such as approximately less than 10⁻⁸ M, 10⁻⁹ M or 10⁻¹⁰ M or even lower. In some embodiments, such antibodies that do not cross-react with TIM3 from a non-human species exhibit essentially undetectable binding against these proteins in standard binding assays.

The term “k_{assoc}” or “kₐ”, as used herein, is intended to refer to the association rate of a particular antibody-antigen interaction, whereas the term “k_{dis}” or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e., k_{d}/kₐ) and is expressed as a molar concentration (M). K_{D} values for antibodies can be determined using methods well established in the art. Available methods for determining the K_{D} of an antibody include surface plasmon resonance, a biosensor system such as a BIACORE® system or flow cytometry and Scatchard analysis.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻¹⁰ M or less, or 10⁻⁸ M or less.

The term “EC₅₀” in the context of an in vitro or in vivo assay using an antibody or antigen binding fragment thereof, refers to the concentration of an antibody or an antigen-binding portion thereof that induces a response that is 50% of the maximal response, i.e., halfway between the maximal response and the baseline.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some embodiments, a predicted nonessential amino acid residue in an anti-TIM3 antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some embodiments, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response.

“Immuno stimulating therapy” or “immuno stimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

“T effector” (“T_{eff}”) cells refers to T cells (e.g., CD4⁺ and CD8⁺ T cells) with cytolytic activities as well as T helper (Th) cells, e.g., Th1 cells, which cells secrete cytokines and activate and direct other immune cells, but does not include regulatory T cells (Treg cells). Certain anti-TIM3 antibodies described herein activate T_{eff} cells, e.g., CD4⁺ and CD8⁺ T_{eff} cells and Th1 cells.

An increased ability to stimulate an immune response or the immune system, can result from an enhanced agonist activity of T cell co-stimulatory receptors and/or an enhanced antagonist activity of inhibitory receptors. An increased ability to stimulate an immune response or the immune system can be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, “administering” refers to the physical introduction of a composition comprising a therapeutic agent to a subject, using any of the various methods and delivery systems known to those skilled in the art. Different routes of administration for the anti-TIM3 antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In some embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8⁺ T cells.

As used herein, the terms “inhibits” or “blocks” (e.g., referring to inhibition/blocking of binding of TIM3-L to TIM3 on cells) are used interchangeably and encompass both partial and complete inhibition/blocking. In some embodiments, the anti-TIM3 antibody inhibits binding of TIM3-L to TIM3 by at least about 50%, for example, about 60%, 70%, 80%, 90%, 95%, 99%, or 100%, determined, e.g., as further described herein. In some embodiments, the anti-TIM3 antibody inhibits binding of TIM3-L to TIM3 by no more than 50%, for example, by about 40%, 30%, 20%, 10%, 5% or 1%, determined, e.g., as further described herein.

As used herein, the phrase “inhibits growth of a tumor” includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

As used herein, “cancer” refers a broad group of diseases characterized by the uncontrolled growth of abnormal cells in the body. A “cancer” or “cancer tissue” can include a tumor. Unregulated cell division can result in the formation of malignant tumors or cells that invade neighboring tissues and can metastasize to distant parts of the body through the lymphatic system or bloodstream. Following metastasis, the distal tumors can be said to be “derived from” the pre-metastasis tumor. For example, a “tumor derived from” a melanoma refers to a tumor that is the result of a metastasized melanoma. Because the distal tumor is derived from the pre-metastasis tumor, the “derived from” tumor can also comprise the pre-metastasis tumor, e.g., a tumor derived from a melanoma can comprise a melanoma. In some embodiments, the cancer or tumor comprises a solid tumor. In some embodiments, the cancer or tumor comprises a solid tumor that is advanced. In some embodiments, the cancer or tumor comprises a solid tumor that has spread. In some embodiments, the cancer or tumor comprises an advanced malignant tumor. In some embodiments, the cancer or tumor is a metastatic cancer or tumor (e.g., stage 4 cancer or tumor).

The terms “treat,” “treating,” “treatment,” and “therapy,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

As used herein, the term “standard-of-care” is used to refer to a treatment process that an ordinary skilled prudent physician uses to treat a certain disease, such as cancer. The standard-of care can vary depending on the type and stage of cancer, the patient's condition and treatment history, and the like, and will be apparent to those skilled in the art.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-i” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that promotes cancer regression in a subject. In some embodiments, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some embodiments, a therapeutically effective amount or dosage of the drug completely inhibits cell growth or tumor growth, i.e., inhibits cell growth or tumor growth by 100%. The ability of a compound to inhibit tumor growth can be evaluated using the assays described herein. Alternatively, this property of a composition can be evaluated by examining the ability of the compound to inhibit cell growth, such inhibition can be measured in vitro by assays known to the skilled practitioner. In some embodiments described herein, tumor regression can be observed and continue for a period of at least about 20, 30, 40, 50, or 60 days. Notwithstanding these ultimate measurements of therapeutic effectiveness, evaluation of immunotherapeutic drugs must also make allowance for “immune-related response patterns.”

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

The term “patient” refers to a human subject that receives either prophylactic or therapeutic treatment.

As used herein, the term “subject” refers to a human. In some embodiments, the subject is treatment naïve. In some embodiments, the subject has received at least one prior therapy for the treatment of a cancer or tumor. In some embodiments, the subject has received, and then progressed, relapsed, or been intolerant to at least one standard treatment regimen. Various standard of care therapies are known in the art for particular types of cancers or tumors. In some embodiments, the at least one standard treatment regimen comprises a treatment in the advanced or metastatic setting according to solid tumor histology.

The use of the term “flat dose” with regard to the methods and dosages described herein means a dose that is administered to a patient without regard for the weight or body surface area (BSA) of the patient. The flat dose is therefore not provided as a mg/kg dose, but rather as an absolute amount of the agent (e.g., the anti-TIM3 antibody). For example, a 60 kg person and a 100 kg person would receive the same dose of an antibody (e.g., 480 mg of an anti-TIM3 antibody).

The term “weight based” dose or dosing as referred to herein means that a dose that is administered to a patient is calculated based on the weight of the patient. For example, when a patient with 60 kg body weight requires 3 mg/kg of an anti-TIM3 antibody, one can calculate and use the appropriate amount of the anti-TIM3 antibody (i.e., 180 mg) for administration.

The use of the term “fixed dose” with regard to a method of the disclosure means that two or more different antibodies in a single composition (e.g., anti-TIM3 antibody and a second antibody, e.g., an anti-PD-1 or anti-PD-L1 antibody) are present in the composition in particular (fixed) ratios with each other. In some embodiments, the fixed dose is based on the weight (e.g., mg) of the antibodies. In some embodiments, the fixed dose is based on the concentration (e.g., mg/ml) of the antibodies.

As used herein, the terms “ug” and “uM” are used interchangeably with “μg” and “μM,” respectively.

Various aspects described herein are described in further detail in the following subsections.

A list of abbreviations in provided in Table 1.

TABLE 1
List of Abbreviations

| Term | Definition |
|---|---|
| Ab | Antibody |
| ADA | anti-drug antibody |
| ADCC | antibody-dependent cell-mediated cytotoxicity |
| ADCP | antibody-dependent cellular phagocytosis |
| AE | adverse event |
| AI | accumulation index |
| AI_AUC | accumulation index ratio of AUC at steady state |
| | to that after the first dose |
| ALP | alkaline phosphatase |
| ALT | alanine aminotransferase |
| ART | antiretroviral therapy |
| AST | aspartate aminotransferase |
| AT | aminotransaminases |
| AUC | area under the concentration-time curve (exposure) |
| AUC(0-T) | area under the concentration-time curve from time |
| | zero to the time of the last |
| | quantifiable concentration |
| AUC(TAU) | area under the concentration-time curve in one |
| | dosing interval |
| BCR | B-cell receptor |
| BICR | blinded independent central review |
| BLRM | Bayesian Logistic Regression Model |
| BRAF | B-Raf proto-oncogene |
| BMS | Bristol-Myers Squibb (i.e., Applicant) |
| BOR | best overall response |
| C | Cycle |
| Cav-gss | steady-state exposures |
| CDC | complement-dependent-cytotoxicity |
| Ceoi | concentration at the end of infusion |
| CI | confidence interval |
| CLT | total body clearance |
| CLTs | total body serum clearance |

### I. Methods of the Disclosure

The present disclosure is directed to a method for treating a tumor or a subject having a cancer or a tumor comprising administering to the subject a therapeutically effective dose of an antibody that binds specifically to a human T-cell immunoglobulin and mucin-domain containing-3 (TIM3) and, e.g., inhibits TIM3 activity (“anti-TIM3 antibody”), e.g., TIM3.18, as a monotherapy or in combination with a PD-1/PD-L1 pathway inhibitor.

In some embodiments, the subject for the present methods is a subject having a solid cancer or solid tumor. In some embodiments, the subject for the present methods is a subject having a solid tumor that is advanced or metastatic. In some embodiments, the subject for the present methods is a subject having advanced cancer that is metastatic, recurrent and/or unresectable. In some embodiments, the subject for the present methods is a subject having cancer that is refractory to (i.e., has not responded to or is resistant to), or has progressed on or after, an immuno-oncology therapy. In some embodiments, the subject for the present methods is a subject having cancer that is resistant to, or has progressed on, an anti-PD1/PD-L1 agent therapy. In some embodiments, the subject for the present methods is a subject having cancer that is refractory to (i.e., has not responded to or is resistant to), or has progressed on, an anti-PD1/PD-L1 agent therapy alone or combined with another agent, e.g., another immuno-oncology agent.

In some embodiments, the present disclosure includes a method of treating a subject having a cancer or a tumor, comprising administering to the subject a therapeutically effective dose of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the anti-TIM3 antibody is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure is directed to a method of treating a subject having a solid cancer or solid tumor, comprising administering to the subject a therapeutically effective amount of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the TIM3 Ab is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure includes a method of treating a subject having a solid tumor that is advanced or metastatic, comprising administering to the subject a therapeutically effective amount of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the anti-TIM3 antibody is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure is directed to a method of treating a subject having advanced cancer that is metastatic, recurrent, and/or unresectable, comprising administering to the subject a therapeutically effective amount of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the anti-TIM3 antibody is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure includes a method of treating a subject having cancer that is refractory to (i.e., has not responded to or is resistant to), or has progressed on or after, an immuno-oncology therapy, comprising administering to the subject a therapeutically effective amount of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the anti-TIM3 antibody is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure is directed to a method of treating a subject having cancer that is resistant to, or has progressed on, an anti-PD1/PD-L1 agent therapy, comprising administering to the subject a therapeutically effective amount of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the anti-TIM3 antibody is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure includes a method of treating a subject having a cancer that is refractory to (i.e., has not responded to or is resistant to), or has progressed on, an anti-PD1/PD-L1 agent therapy alone or combined with another agent, e.g., another immuno-oncology agent, comprising administering to the subject a therapeutically effective amount of an anti-TIM3 antibody, e.g., TIM3.18.IgG1, TIM3.18.IgG1.1, TIM3.18.IgG1.3 or TIM3.18.IgG4, as a monotherapy or in combination with an PD1/PD-L1 pathway inhibitor, wherein the anti-TIM3 antibody is administered every 1, 2, 3, 4 or 5 weeks. In some embodiments, the PD1/PD-L1 pathway inhibitor administered in combination with an anti-TIM3 antibody is administered every 1, 2, 3, 4 or 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 every 2 weeks.

In some embodiments, the present disclosure provides a method of treating cancer (e.g., solid tumor, such as breast, lung, renal, colon, colorectal, liver, melanoma or head and neck) in a subject, comprising administering to the subject 8 mg of an anti-TIM3 antibody comprising the VH and VL domains of TIM3.18 every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 every 2 weeks.

In some embodiments, the present disclosures is directed to a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 every 4 weeks.

In some embodiments, disclosed herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 every 4 weeks.

In some embodiments, disclosed herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 every 5 weeks.

In some embodiments, the present disclosure includes method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 every 5 weeks

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG1 every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1 every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 140 mg of TIM3.18.IgG1 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1 every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1 every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1 every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG1 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 8 mg of TIM3.18.IgG1 every 3 weeks

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1 every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1 every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of TIM3.18.IgG1 every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 72 mg of TIM3.18.IgG1 every 3 weeks.

In some embodiments, the present application is directed to a method of treating cancer (e.g., solid tumor, such as breast, lung, renal, colon, colorectal, liver, melanoma or head and neck cancer) in a subject, comprising administering to the subject 140 mg of TIM3.18.IgG1 every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1 every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1 every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 800 mg of TIM3.18.IgG1 every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1 every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1 every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1 every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 24 mg of TIM3.18.IgG1 every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of TIM3.18.IgG1 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1 every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 140 mg of TIM3.18.IgG1 every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 200 mg of TIM3.18.IgG1 every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 350 mg of TIM3.18.IgG1 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1 every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1 every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1 every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1 every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1 every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1 every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1 every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1 every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1 every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1 every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1 every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1 every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG1.1f every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.1f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.1f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.1f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.1f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.1f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 140 mg of TIM3.18.IgG1.1f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1.1f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1.1f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.1f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.1f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.1f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.1f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG1.1f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 8 mg of TIM3.18.IgG1.1f every 3 weeks

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.1f every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of TIM3.18.IgG1.1f every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 72 mg of TIM3.18.IgG1.1f every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1.1f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.1f every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.1f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.1f every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 800 mg of TIM3.18.IgG1.1f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.1f every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.1f every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.1f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.1f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of TIM3.18.IgG1.1f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.1f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 140 mg of TIM3.18.IgG1.1f every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 200 mg of TIM3.18.IgG1.1f every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 350 mg of TIM3.18.IgG1.1f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.1f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.1f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.1f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.1f every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.1f every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.1f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 72 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1.1f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.1f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.1f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG1.3f every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.3f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.3f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.3f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.3f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 72 mg of TIM3.18.IgG1.3f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 140 mg of TIM3.18.IgG1.3f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1.3f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1.3f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.3f every 2 weeks.

In some embodiments, the present disclosure is provides a method of treating cancer (e.g., solid tumor, such as breast, lung, renal, colon, colorectal, liver, melanoma or head and neck cancer) in a subject, comprising administering to the subject 600 mg of TIM3.18.IgG1.3f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.3f every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.3f every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG1.3f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 8 mg of TIM3.18.IgG1.3f every 3 weeks

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.3f every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of TIM3.18.IgG1.3f every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 72 mg of TIM3.18.IgG1.3f every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG1.3f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.3f every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.3f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.3f every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 800 mg of TIM3.18.IgG1.3f every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.3f every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.3f every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.3f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.3f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of TIM3.18.IgG1.3f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.3f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 200 mg of TIM3.18.IgG1.3f every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 350 mg of TIM3.18.IgG1.3f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.3f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.3f every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.3f every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.3f every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.3f every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.3f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 72 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f every 5 weeks.

In some embodiments, the present disclosure includes method of treating cancer (e.g., solid tumor, such as breast, lung, renal, colon, colorectal, liver, melanoma or head and neck cancer) in a subject, comprising administering to the subject 200 mg of TIM3.18.IgG1.3f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.3f every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.3f every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG4P every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG4P every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG4P every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG4P every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG4P every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG4P every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 140 mg of TIM3.18.IgG4P every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG4P every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG4P every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG4P every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG4P every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG4P every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG4P every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of TIM3.18.IgG4P every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 8 mg of TIM3.18.IgG4P every 3 weeks

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG4P every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG4P every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of TIM3.18.IgG4P every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 72 mg of TIM3.18.IgG4P every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 140 mg of TIM3.18.IgG4P every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG4P every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG4P every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG4P every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG4P every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 800 mg of TIM3.18.IgG4P every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG4P every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG4P every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG4P every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG4P every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 24 mg of TIM3.18.IgG4P every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of TIM3.18.IgG4P every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG4P every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG4P every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 200 mg of TIM3.18.IgG4P every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 350 mg of TIM3.18.IgG4P every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG4P every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG4P every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG4P every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG4P every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG4P every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG4P every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG4P every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG4P every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG4P every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG4P every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG4P every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of TIM3.18.IgG4P every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG4P every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG4P every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG4P every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG4P every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG4P every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, every 2 weeks.

In some embodiments, the present application includes a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, every 2 weeks.

In some embodiments, the present application provides a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, every 2 weeks.

In some embodiments, the present application provides a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, every 2 weeks.

In some embodiments, the present application is directed to a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, every 3 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, every 3 weeks.

In some embodiments, the present application includes a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, every 4 weeks.

In some embodiments, the present application includes a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, every 4 weeks.

In some embodiments, the present application includes a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, every 4 weeks.

In some embodiments, the present application provides a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, at 240 mg every 2 weeks.

In some embodiments, disclosed herein is a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, at 360 mg every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, at 360 mg every 3 weeks.

In some embodiments, disclosed herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, at 360 mg every 3 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, at 480 mg every 4 weeks.

In some embodiments provided herein is a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of an anti-TIM3 antibody comprising the VH and VL domains of TIM3.18 and a PD-1/PD-L1 antagonist, e.g., nivolumab, at 600 mg every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of an anti-TIM3 antibody comprising the VH and VL domains of TIM3.18 and a PD-1/PD-L1 antagonist, e.g., nivolumab, at 600 mg every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 24 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 48 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 72 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 140 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 200 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 350 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 480 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 600 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 800 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of an anti-TIM3 antibody comprising the VH and VL domains of TIM3.18 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, at 480 mg every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, at 480 mg every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG1 and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG1 and a PD-1/PD-L1 antagonist, e.g., nivolumab, at 600 mg every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG1 and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG1 and a PD-1/PD-L1 antagonist, e.g., nivolumab, at 600 mg every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG1 and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1 and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1 and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1 and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG1 and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1 and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1 and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1 and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1 and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, at 480 mg every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, at 480 mg every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG1.1f and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG1.1f and a PD-1/PD-L1 antagonist, e.g., nivolumab, at 600 mg every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG1.1f and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG1.1f and a PD-1/PD-L1 antagonist, e.g., nivolumab, at 600 mg every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG1.1f and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.1f and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.1f and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1.1f and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG1.1f and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.1f and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.1f and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1.1f and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.1f and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, at 480 mg every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, at 480 mg every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG1.3f and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG1.3f and a PD-1/PD-L1 antagonist, e.g., nivolumab, at 600 mg every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG1.3f and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG1.3f and a PD-1/PD-L1 antagonist, e.g., nivolumab, at 600 mg every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG1.3f and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG1.3f and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG1.3f and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG1.3f and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG1.3f and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG1.3f and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG1.3f and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG1.3f and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG1.3f and a PD-1/PD-L1 antagonist, e.g., nivolumab, at 600 mg every 5 weeks.

In some embodiments, provided herein is a method of treating cancer (e.g., solid tumor, such as breast, lung, renal, colon, colorectal, liver, melanoma or head and neck cancer) in a subject, comprising administering to the subject 4 mg of TIM3.18.IgG4P and a PD-1/PD-L1 antagonist, e.g., nivolumab, at 240 mg every 2 weeks.

In some embodiments, provided herein is a method of treating cancer (e.g., solid tumor, such as breast, lung, renal, colon, colorectal, liver, melanoma or head and neck cancer) in a subject, comprising administering to the subject 8 mg of TIM3.18.IgG4P and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 16 mg of TIM3.18.IgG4P and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG4P and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG4P and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG4P and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG4P and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG4P and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG4P and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG4P and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 600 mg of TIM3.18.IgG4P and a PD-1/PD-L1 antagonist, e.g., nivolumab, at 240 mg every 2 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG4P and a PD-1/PD-L1 antagonist, e.g., nivolumab, at 240 mg every 2 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG4P and a PD-1/PD-L1 antagonist, e.g., nivolumab, at 240 mg every 2 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 4 mg of TIM3.18.IgG4P and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 8 mg of TIM3.18.IgG4P and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG4P and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 24 mg of TIM3.18.IgG4P and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 48 mg of TIM3.18.IgG4P and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG4P and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG4P and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG4P and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 350 mg of TIM3.18.IgG4P and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 480 mg of TIM3.18.IgG4P and a PD-1/PD-L1 antagonist, e.g., nivolumab, at 360 mg every 3 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 600 mg of TIM3.18.IgG4P and a PD-1/PD-L1 antagonist, e.g., nivolumab, at 360 mg every 3 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 800 mg of TIM3.18.IgG4P and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 1000 mg of TIM3.18.IgG4P and a PD-1/PD-L1 antagonist, e.g., nivolumab, at 360 mg every 3 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 4 mg of TIM3.18.IgG4P and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 8 mg of TIM3.18.IgG4P and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 16 mg of TIM3.18.IgG4P and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 24 mg of TIM3.18.IgG4P and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 48 mg of TIM3.18.IgG4P and a PD-1/PD-L1 antagonist, e.g., nivolumab, at 480 mg every 4 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 72 mg of TIM3.18.IgG4P and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG4P and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 200 mg of TIM3.18.IgG4P and a PD-1/PD-L1 antagonist, e.g., nivolumab, at 480 mg every 4 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 350 mg of TIM3.18.IgG4P and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 480 mg of TIM3.18.IgG4P and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG4P and a PD-1/PD-L1 antagonist, e.g., nivolumab, at 480 mg every 4 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 800 mg of TIM3.18.IgG4P and a PD-1/PD-L1 antagonist, e.g., nivolumab, at 480 mg every 4 weeks.

In some embodiments, provided herein is a method of treating cancer (e.g., solid tumor, such as breast, lung, renal, colon, colorectal, liver, melanoma or head and neck cancer) in a subject, comprising administering to the subject 1000 mg of TIM3.18.IgG4P and a PD-1/PD-L1 antagonist, e.g., nivolumab, at 480 mg every 4 weeks.

In some embodiments, the present application discloses a method of treating cancer (e.g., solid tumor, such as breast, lung, renal, colon, colorectal, liver, melanoma or head and neck cancer) in a subject, comprising administering to the subject 4 mg of TIM3.18.IgG4P and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 8 mg of TIM3.18.IgG4P and a PD-1/PD-L1 antagonist, e.g., nivolumab, at 600 mg every 5 weeks.

In some embodiments, disclosed herein is a method of treating cancer (e.g., solid tumor, such as breast, lung, renal, colon, colorectal, liver, melanoma or head and neck cancer) in a subject, comprising administering to the subject 16 mg of TIM3.18.IgG4P and a PD-1/PD-L1 antagonist, e.g., nivolumab, at 600 mg every 5 weeks.

In some embodiments, the present application provides a method of treating cancer (e.g., solid tumor, such as breast, lung, renal, colon, colorectal, liver, melanoma or head and neck cancer) in a subject, comprising administering to the subject 24 mg of TIM3.18.IgG4P and a PD-1/PD-L1 antagonist, e.g., nivolumab, at 600 mg every 5 weeks.

In some embodiments, the present application includes a method of treating cancer (e.g., solid tumor, such as breast, lung, renal, colon, colorectal, liver, melanoma or head and neck cancer) in a subject, comprising administering to the subject 48 mg of TIM3.18.IgG4P and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 72 mg of TIM3.18.IgG4P and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 140 mg of TIM3.18.IgG4P and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 200 mg of TIM3.18.IgG4P and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 350 mg of TIM3.18.IgG4P and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 480 mg of TIM3.18.IgG4P and a PD-1/PD-L1 antagonist, e.g., nivolumab, at 600 mg every 5 weeks.

In some embodiments, the present disclosure includes a method of treating cancer (e.g., solid tumor, such as breast, lung, renal, colon, colorectal, liver, melanoma or head and neck cancer) in a subject, comprising administering to the subject 600 mg of TIM3.18.IgG4P and a PD-1/PD-L1 antagonist, e.g., nivolumab, at 600 mg every 5 weeks.

In some embodiments, the present disclosure is directed to a method of treating cancer (e.g., solid tumor, such as breast, lung, renal, colon, colorectal, liver, melanoma or head and neck cancer) in a subject, comprising administering to the subject 800 mg of TIM3.18.IgG4P and a PD-1/PD-L1 antagonist, e.g., nivolumab, at 600 mg every 5 weeks.

In some embodiments, the present disclosure provides a method of treating cancer (e.g., solid tumor, such as breast, lung, renal, colon, colorectal, liver, melanoma or head and neck cancer) in a subject, comprising administering to the subject 1000 mg of TIM3.18.IgG4P and a PD-1/PD-L1 antagonist, e.g., nivolumab, at 600 mg every 5 weeks.

In some embodiments, TIM3.18.IgG1.1f comprises a heavy chain comprising SEQ ID NOs: 349 or 350 and a light chain comprising SEQ ID NO: 29. In some embodiments, TIM3.18.IgG1.3f comprises a heavy chain comprising SEQ ID NOs: 351 or 352 and a light chain comprising SEQ ID NO: 29. In some embodiments, TIM3.18.IgG4P comprises a heavy chain comprising SEQ ID NOs: 353 or 354 and a light chain comprising SEQ ID NO: 29.

In some embodiments, the VH domain of TIM3.18 comprises the amino acid sequence: QLQLQESGPGLVKPSETLSLTCTVSGGSISSRSYYWGWIRQPPGKGLEWIGSIYYSGFTY YQPSLKSRVTISVDTSKNQFSLKLSSVTAADTAVYYCATGGPYGDYAHWFEPWGQGTLVTVSS (SEQ ID NO: 364). In some embodiments, the VL domain of TIM3.18 comprises the amino acid sequence:

(SEQ ID NO: 60)
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPIT
FGQGTRLEIK.

“TIM3.18.IgG4” and “TIM3.18.IgG4P” are used interchangeably herein.

In some embodiments, the presently described immunotherapy can be used to treat a patient suffering from any condition that can be remedied or prevented by this method. Exemplary conditions are cancers, such as, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer (e.g., triple negative breast cancer), ovarian cancer, prostate cancer, colorectal cancer, non-small cell lung cancer (NSCLC), squamous cell cancer, basal cell cancer, carcinoma of the head and neck (e.g., squamous carcinoma of the head and neck), adenocarcinoma, sweat gland cancer, sebaceous gland cancer, papillary cancer, papillary adenocarcinomas, cystadenocarcinoma, medullary cancer, bronchogenic cancer, renal cell cancer (e.g., renal cell carcinoma), hepatoma, bile duct cancer, choriocarcinoma, seminoma, embryonal cancer, Wilms' tumor, cervical cancer, testicular cancer, lung cancer, small cell lung cancer, bladder cancer (e.g., urothelial carcinoma), epithelial cancer, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma and leukemias.

In any of the above methods, the cancer can be a bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, virus-related cancer, or any combination thereof.

In some embodiments, the anti-TIM3 antibody interacts with soluble human TIM3. In some embodiments, the anti-TIM3 antibody interacts with human TIM3 expressed on the surface of a cell, wherein the cell is an immune cell. In some embodiments, the cell is selected from the group consisting of a monocyte, macrophage, dendritic cell (DC), NK cell, CD4⁺ T cell, CD8⁺ T cells, and any combination thereof. In some embodiments, the anti-TIM3 antibody induces or enhances T cell activation, as evidenced by (i) increased IFN-γ production in TIM3-expressing T cells (e.g., Th1 cells or TILs) and/or (ii) enhanced proliferation of TIM3-expressing T cells (e.g., Th1 cells or TILs). In some embodiments, the anti-TIM3 antibody suppresses or does not promote clonal expansion of CD4⁺ regulatory T cells. In some embodiments, the anti-TIM3 antibody promotes an anti-tumor immune response by increasing the effector activity (e.g., increased IFN-γ production) of the T cells. In some embodiments, the anti-TIM3 antibody promotes an anti-tumor immune response by increasing the number of the T cells (e.g., Th1 cells or TILs). In some embodiments, the anti-TIM3 antibody promotes an anti-tumor immune response by suppressing an immunosuppressive response by suppressing the expansion of CD4⁺ regulatory T cells.

In some embodiments, the subject has received one, two, three, four, five, or more prior cancer treatments. In some embodiments, the subject has progressed on other cancer treatments. In some embodiments, the prior cancer treatment comprised an anti-angiogenic therapy regimen (e.g., sunitinib, sorafenib, pazopanib, axitinib, tivozanib, bevacizumab, or any combination thereof), e.g., for renal cell carcinoma. In some embodiments, the prior cancer treatment comprised a standard systemic therapy for metastatic and/or unresectable disease (e.g., Oxaliplatin, Irinotecan, or the combination thereof), e.g., for colorectal cancer. In some embodiments, the prior cancer treatment comprised a platinum-based chemotherapy, e.g., for NSCLC. In some embodiments, the tumor is advanced, recurring, metastatic, and/or refractory.

In some embodiments, the subject has renal cell carcinoma (RCC) and, e.g., has received and progressed on or after an anti-PD-1 therapy (combined or not with other immuno-oncology agents). In some embodiments, the subject has microsatellite instability high (MSI-H) colorectal carcinoma (CRC) and, e.g., has received and progressed on or after anti-PD-1 therapy. In some embodiments, the subject has microsatellite stable (MSS) CRC. In some embodiments, the subject has non-small cell lung carcinoma (NSCLC) and, e.g., has received and progressed on or after anti-PD-1 therapy. In some embodiments, the subject has squamous cell carcinoma of the head and neck (SCCHN) and, e.g., has received and progressed on or after anti-PD-1 therapy.

In some embodiments, the RCC subject must have received at least one but not more than two prior anti-angiogenic therapy regimens (including but not limited to sunitinib, sorafenib, pazopanib, axitinib, tivozanib, and bevacizumab) in the advanced or metastatic setting. In some embodiments, the RCC subject has received prior cytokine therapy (e.g., IL-2 or IFN-γ), vaccine therapy, or treatment with cytotoxics. In some embodiments, the RCC subject has previously received anti-PD-L1 therapy (with or without other immuno-oncology agents). In some embodiments, the RCC subject has not previously received an anti-PD-L1 therapy (i.e., anti-PD-L1 therapy naïve).

In some embodiments, the CRC subject must have received and then progressed on or after, or have been intolerant or refractory to, at least 1 standard systemic therapy for metastatic and/or unresectable disease (or have progressed within 6 months of adjuvant therapy), including Oxaliplatin and Irinotecan. In some embodiments, the CRC subject must know the microsatellite instability (MSI) or mismatch repair (MMR) status. In some embodiments, the CRC subject knows his or her V-Ki-ras2 Kirsten rat sarcoma viral oncogene homolog (KRAS), and B-Raf proto-oncogene (BRAF) status. In some embodiments, the CRC subject has received prior anti-angiogenic therapy (e.g., bevacizumab) and/or anti-epidermal growth factor receptor therapy (e.g., cetuximab or panitumumab).

In some embodiments, the therapy of the present disclosure (e.g., administration of an anti-TIM3 antibody) effectively increases the duration of survival of the subject. For example, the duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more (e.g., about 2, 3, 4, or 5 years) when compared to another subject not treated with the anti-TIM3 antibody (e.g., treated with chemotherapy alone).

In some embodiments, the therapy of the present disclosure effectively increases the duration of progression-free survival of the subject. For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year (e.g., about 2, 3, 4, or 5 years) when compared to another subject not treated with the anti-TIM3 antibody (e.g., treated with chemotherapy alone).

In some embodiments, the therapy of the present disclosure effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to another group of subjects not treated with the anti-TIM3 antibody (e.g., treated with chemotherapy alone).

In some embodiments, the methods comprise administering an effective amount of an anti-TIM3 antibody, alone or in combination with an inhibitor of the PD-1 signaling pathway (e.g., an anti-PD-1 antibody or an anti-PD-L1 antibody), to a subject in need thereof. An effective amount of an anti-TIM3 antibody and/or an anti-PD-1 antibody can be a flat dose, a weight based dose, or both. Dosage regimens are adjusted to provide the optimum desired response, e.g., a maximal therapeutic response and/or minimal adverse effects. While the subsequent disclosure discloses dosing for anti-PD-1 antibodies, such disclosure can be equally applicable to anti-PD-L1 antibodies.

In some embodiments, the anti-TIM3 antibody for either monotherapy or combination therapy is administered at a flat dose, e.g., 1 mg to 960 mg. In some embodiments, the flat dose ranges from about 2 mg to about 800 mg, about 4 mg to about 800 mg, about 8 mg to about 800 mg, about 16 mg to about 800 mg, about 24 mg to about 800 mg, about 36 mg to about 800 mg, about 40 mg to about 800 mg, about 48 mg to about 800 mg, about 54 mg to about 800 mg, about 64 mg to about 800 mg, about 72 mg to about 800 mg, about 80 mg to about 800 mg, about 100 mg to about 800 mg, about 150 mg to about 800 mg, about 160 mg to about 800 mg, about 180 mg to about 800 mg, about 200 mg to about 800 mg, about 240 mg to about 800 mg, about 300 mg to about 800 mg, about 320 mg to about 800 mg, about 350 mg to about 800 mg, about 360 mg to about 800 mg, about 380 mg to about 800 mg, about 400 mg to about 800 mg, about 420 mg to about 800 mg, about 440 mg to about 800 mg, about 450 mg to about 800 mg, about 460 mg to about 800 mg, about 480 mg to about 800 mg, or about 500 mg to about 800 mg. In some embodiments, the flat dose ranges from about 2 mg to about 640 mg, about 4 mg to about 640 mg, about 8 mg to about 640 mg, about 16 mg to about 640 mg, about 24 mg to about 640 mg, about 36 mg to about 640 mg, about 40 mg to about 640 mg, about 48 mg to about 640 mg, about 54 mg to about 640 mg, about 64 mg to about 640 mg, about 72 mg to about 640 mg, about 80 mg to about 640 mg, about 100 mg to about 640 mg, about 150 mg to about 640 mg, about 160 mg to about 640 mg, about 180 mg to about 640 mg, about 200 mg to about 640 mg, about 240 mg to about 640 mg, about 300 mg to about 640 mg, about 320 mg to about 640 mg, about 350 mg to about 640 mg, about 360 mg to about 640 mg, about 380 mg to about 640 mg, about 400 mg to about 640 mg, about 420 mg to about 640 mg, about 440 mg to about 640 mg, about 450 mg to about 640 mg, about 460 mg to about 640 mg, about 480 mg to about 640 mg, about 500 mg to about 640 mg, about 540 mg to about 640 mg, about 560 mg to about 640 mg. In some embodiments, the flat dose ranges from about 2 mg to about 500 mg, about 4 mg to about 500 mg, about 8 mg to about 500 mg, about 16 mg to about 500 mg, about 24 mg to about 500 mg, about 36 mg to about 500 mg, about 40 mg to about 500 mg, about 48 mg to about 500 mg, about 54 mg to about 500 mg, about 64 mg to about 500 mg, about 72 mg to about 500 mg, about 80 mg to about 500 mg, about 100 mg to about 500 mg, about 150 mg to about 500 mg, about 160 mg to about 500 mg, about 180 mg to about 500 mg, about 200 mg to about 500 mg, about 240 mg to about 500 mg, about 300 mg to about 500 mg, about 320 mg to about 500 mg, about 350 mg to about 500 mg, about 360 mg to about 500 mg, about 380 mg to about 500 mg, about 400 mg to about 500 mg, about 420 mg to about 500 mg, about 440 mg to about 500 mg, about 450 mg to about 500 mg, about 460 mg to about 500 mg, or about 480 mg to about 500 mg. In some embodiments, the flat dose ranges from about 2 mg to about 480 mg, about 4 mg to about 480 mg, about 8 mg to about 480 mg, about 16 mg to about 480 mg, about 24 mg to about 480 mg, about 36 mg to about 480 mg, about 40 mg to about 480 mg, about 48 mg to about 480 mg, about 54 mg to about 480 mg, about 64 mg to about 480 mg, about 72 mg to about 480 mg, about 80 mg to about 480 mg, about 100 mg to about 480 mg, about 150 mg to about 480 mg, about 160 mg to about 480 mg, about 180 mg to about 480 mg, about 200 mg to about 480 mg, about 240 mg to about 480 mg, about 300 mg to about 480 mg, about 320 mg to about 480 mg, about 350 mg to about 480 mg, about 360 mg to about 480 mg, about 380 mg to about 480 mg, about 400 mg to about 480 mg, about 420 mg to about 480 mg, about 440 mg to about 480 mg, about 450 mg to about 480 mg, or about 460 mg to about 480 mg. In some embodiments, the flat dose ranges from about 240 mg to about 480 mg, about 360 mg to about 480 mg, about 400 mg to about 500 mg, about 300 mg to about 500 mg, or about 300 mg to about 400 mg.

In some embodiments, the anti-TIM3 antibody for either monotherapy or combination therapy is administered at a flat dose of about 2 mg, about 4 mg, 8 mg, about 10 mg, about 16 mg, about 20 mg, about 24 mg, about 30 mg, about 36 mg, about 40 mg, about 48 mg, about 50 mg, about 54 mg, about 64 mg, about 72 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 700 mg, about 720 mg, about 750 mg, about 760 mg, or about 800 mg. In some embodiments, the flat dose is about 240 mg, about 360 mg, or about 480 mg. In some embodiments, the flat dose is about 240 mg. In some embodiments, the flat dose is about 360 mg. In some embodiments, the flat dose is about 480 mg.

In some embodiments, the anti-TIM3 antibody is administered at a flat dose of 1 mg, 4 mg, 8 mg, 24 mg, 72 mg, 150 mg, 240 mg, 480 mg, 600 mg, 800 mg, or 1000 mg every 3, 4, or 5 weeks.

In some embodiments, the anti-TIM3 antibody is administered at a flat dose of 1 mg, 4 mg, 8 mg, 24 mg, 72 mg, 150 mg, 240 mg, 480 mg, 600 mg, 800 mg, or 1000 mg every 3, 4, or 5 weeks in combination with an PD1/PD-L1 pathway inhibitor that is administered at a flat dose of 120 mg, 240 mg, or 480 mg every 2, 3 or 4 weeks, respectively.

In some embodiments, the anti-TIM3 antibody is administered at a flat dose of 1 mg, 4 mg, 8 mg, 24 mg, 72 mg, 150 mg, 240 mg, 480 mg, 600 mg, 800 mg, or 1000 mg every 4 weeks in combination with an PD1/PD-L1 pathway inhibitor that is administered at a flat dose of 480 mg every 4 weeks, and wherein the anti-TIM3 antibody and the PD1/PD-L1 pathway inhibitor are administered i.v. to the subject on the same day.

In some embodiments, the anti-TIM3 antibody is administered at a flat dose of 1 mg, 4, 8, 24, 72, 150, 240, 480, 600, 800 mg, or 1000 mg every 4 weeks in combination with an PD1/PD-L1 pathway inhibitor that is administered at a flat dose of 480 mg every 4 weeks, and wherein the anti-TIM3 antibody and the PD1/PD-L1 pathway inhibitor are administered i.v. to the subject on the same day, and wherein the PD1/PD-L1 pathway inhibitor is administered before the anti-TIM3 antibody.

In some embodiments, the anti-TIM3 antibody for either monotherapy or combination therapy is administered at a weight-based dose. In some embodiments, the weight-based dose ranges from about 0.0125 mg/kg to about 10 mg/kg, about 0.025 mg/kg to about 10 mg/kg, about 0.05 mg/kg to about 10 mg/kg, about 0.1 mg/kg to about 10 mg/kg, about 0.3 mg/kg to about 10 mg/kg, 0.9 mg/kg to about 10 mg/kg, about 1 mg/kg to about 10 mg/kg, about 1.5 mg/kg to about 10 mg/kg, about 2 mg/kg to about 10 mg/kg, about 2.5 mg/kg to about 10 mg/kg, about 3 mg/kg to about 10 mg/kg, about 3.5 mg/kg to about 10 mg/kg, about 4 mg/kg to about 10 mg/kg, about 4.5 mg/kg to about 10 mg/kg, about 5 mg/kg to about 10 mg/kg, about 5.5 mg/kg to about 10 mg/kg, about 6 mg/kg to about 10 mg/kg, about 6.5 mg/kg to about 10 mg/kg, about 7 mg/kg to about 10 mg/kg, about 7.5 mg/kg to about 10 mg/kg, about 8 mg/kg to about 10 mg/kg, about 8.5 mg/kg to about 10 mg/kg, about 9 mg/kg to about 10 mg/kg, or about 9.5 mg/kg to about 10 mg/kg. In some embodiments, the weight-based dose ranges from about 0.0125 mg/kg to about 8 mg/kg, about 0.025 mg/kg to about 8 mg/kg, about 0.05 mg/kg to about 8 mg/kg, about 0.1 mg/kg to about 8 mg/kg, about 0.3 mg/kg to about 8 mg/kg, 0.9 mg/kg to about 8 mg/kg, about 1 mg/kg to about 8 mg/kg, about 1.5 mg/kg to about 8 mg/kg, about 2 mg/kg to about 8 mg/kg, about 2.5 mg/kg to about 8 mg/kg, about 3 mg/kg to about 8 mg/kg, about 3.5 mg/kg to about 8 mg/kg, about 4 mg/kg to about 8 mg/kg, about 4.5 mg/kg to about 8 mg/kg, about 5 mg/kg to about 8 mg/kg, about 5.5 mg/kg to about 8 mg/kg, about 6 mg/kg to about 8 mg/kg, about 6.5 mg/kg to about 8 mg/kg, about 7 mg/kg to about 8 mg/kg, or about 7.5 mg/kg to about 8 mg/kg. In some embodiments, the weight-based dose ranges from about 0.0125 mg/kg to about 7 mg/kg, about 0.025 mg/kg to about 7 mg/kg, about 0.05 mg/kg to about 7 mg/kg, about 0.1 mg/kg to about 7 mg/kg, about 0.3 mg/kg to about 7 mg/kg, 0.9 mg/kg to about 7 mg/kg, about 1 mg/kg to about 7 mg/kg, about 1.5 mg/kg to about 7 mg/kg, about 2 mg/kg to about 7 mg/kg, about 2.5 mg/kg to about 7 mg/kg, about 3 mg/kg to about 7 mg/kg, about 3.5 mg/kg to about 7 mg/kg, about 4 mg/kg to about 7 mg/kg, about 4.5 mg/kg to about 7 mg/kg, about 5 mg/kg to about 7 mg/kg, about 5.5 mg/kg to about 7 mg/kg, about 6 mg/kg to about 7 mg/kg, or about 6.5 mg/kg to about 7 mg/kg.

In some embodiments, the weight-based dose ranges from about 0.0125 mg/kg to about 6 mg/kg, about 0.025 mg/kg to about 6 mg/kg, about 0.05 mg/kg to about 6 mg/kg, about 0.1 mg/kg to about 6 mg/kg, about 0.3 mg/kg to about 6 mg/kg, 0.9 mg/kg to about 6 mg/kg, about 1 mg/kg to about 6 mg/kg, about 1.5 mg/kg to about 6 mg/kg, about 2 mg/kg to about 6 mg/kg, about 2.5 mg/kg to about 6 mg/kg, about 3 mg/kg to about 6 mg/kg, about 3.5 mg/kg to about 6 mg/kg, about 4 mg/kg to about 6 mg/kg, about 4.5 mg/kg to about 6 mg/kg, about 5 mg/kg to about 6 mg/kg, or about 5.5 mg/kg to about 6 mg/kg. In some embodiments, the weight-based dose ranges from about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 7 mg/kg, about 6 mg/kg to about 7 mg/kg, or about 6 mg/kg to about 8 mg/kg.

In some embodiments, the anti-TIM3 antibody for either monotherapy or combination therapy is administered at a weight-based dose of about 0.1 mg/kg, about 0.3 mg/kg, about 0.9 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg.

In some embodiments, the anti-TIM3 antibody is administered with a therapeutically effective amount of an anti-PD-1 antibody (e.g., nivolumab). In some embodiments, the anti-PD-1 antibody is administered at a flat dose ranging from about 80 mg to about 640 mg or a weight-based dose ranging from about 1 mg/kg to about 8 mg/kg.

In some embodiments, the anti-PD-1 antibody used with an anti-TIM3 antibody in combination is administered at a flat dose ranging from about 100 mg to about 640 mg, about 120 mg to about 640 mg, about 150 mg to about 640 mg, about 160 mg to about 640 mg, about 180 mg to about 640 mg, about 240 mg to about 640 mg, about 300 mg to about 640 mg, about 320 mg to about 640 mg, about 360 mg to about 640 mg, about 400 mg to about 640 mg, about 420 mg to about 640 mg, about 480 mg to about 640 mg, about 540 mg to about 640 mg, about 560 mg to about 640 mg, about 100 mg to about 560 mg, about 120 mg to about 560 mg, about 150 mg to about 560 mg, about 160 mg to about 560 mg, about 180 mg to about 560 mg, about 240 mg to about 560 mg, about 300 mg to about 560 mg, about 320 mg to about 560 mg, about 360 mg to about 560 mg, about 400 mg to about 560 mg, about 420 mg to about 560 mg, about 480 mg to about 560 mg, about 100 mg to about 500 mg, about 120 mg to about 500 mg, about 150 mg to about 500 mg, about 160 mg to about 500 mg, about 180 mg to about 500 mg, about 240 mg to about 500 mg, about 300 mg to about 500 mg, about 320 mg to about 500 mg, about 360 mg to about 500 mg, about 400 mg to about 500 mg, about 420 mg to about 500 mg, about 450 mg to about 500 mg, about 480 mg to about 500 mg, about 240 mg to about 400 mg, about 300 mg to about 400 mg, about 320 mg to about 400 mg, or about 360 mg to about 400 mg.

In some embodiments, the anti-PD-1 antibody used with an anti-TIM3 antibody in combination is administered at a flat dose of about 100 mg, about 120 mg, about 140 mg, about 160 mg, about 180 mg, about 200 mg, about 240 mg, about 300 mg, about 360 mg, about 420 mg, about 450 mg, about 480 mg, about 500 mg, about 540 mg, about 560 mg, about 600 mg, or about 640 mg.

In some embodiments, the anti-PD-1 antibody used with an anti-TIM3 antibody in combination is administered at a weight-based dose ranging from about 1 mg/kg to about 7 mg/kg, about 1 mg/kg to about 6 mg/kg, about 1 mg/kg to about 5 mg/kg, about 1 mg/kg to about 4 mg/kg, about 1 mg/kg to about 3 mg/kg, about 1 mg/kg to about 2 mg/kg, from about 2 mg/kg to about 7 mg/kg, about 2 mg/kg to about 6 mg/kg, about 2 mg/kg to about 5 mg/kg, about 2 mg/kg to about 4 mg/kg, about 2 mg/kg to about 3 mg/kg, from about 3 mg/kg to about 7 mg/kg, about 3 mg/kg to about 6 mg/kg, about 3 mg/kg to about 5 mg/kg, about 3 mg/kg to about 4 mg/kg, from about 4 mg/kg to about 7 mg/kg, about 4 mg/kg to about 6 mg/kg, about 4 mg/kg to about 5 mg/kg, from about 5 mg/kg to about 7 mg/kg, about 5 mg/kg to about 6 mg/kg, or about 6 mg/kg to about 7 mg/kg.

In some embodiments, the anti-PD-1 antibody used with an anti-TIM3 antibody in combination is administered at a weight-based dose of about 1 mg/kg, about 2 mg/kg, about 3 mg/kg, about 4 mg/kg, about 5 mg/kg, about 6 mg/kg, about 7 mg/kg, or about 8 mg/kg.

In some embodiments, the anti-TIM3 antibody for either monotherapy or combination therapy is administered at a dosing interval of about 1 week, about 2 weeks, about 3 weeks, about 4 weeks, about 5 weeks, or about 6 weeks. In some embodiments, the dosing interval for an anti-TIM3 antibody therapy is about 2 weeks. In some embodiments, the dosing interval for an anti-TIM3 antibody therapy is about 3 weeks. In some embodiments, the dosing interval for an anti-TIM3 antibody therapy is about 4 weeks.

In some embodiments, the anti-PD-1 antibody for combination therapy with an anti-TIM3 antibody is administered at a dosing interval of about 1 week, about 2 weeks, about 3 weeks, about 4 weeks, about 5 weeks, or about 6 weeks. In some embodiments, the dosing interval for an anti-TIM3 antibody therapy is about 2 weeks. In some embodiments, the dosing interval for an anti-TIM3 antibody therapy is about 3 weeks. In some embodiments, the dosing interval for an anti-TIM3 antibody therapy is about 4 weeks.

In some embodiments, the anti-TIM3 antibody and/or the anti-PD-1 antibody is administered via intravenous administration about every four weeks, e.g., for up to 12 cycles (8-week cycles) or until complete response or confirmed progressive disease. In some embodiments, the anti-TIM3 antibody treatment, or any combination treatment disclosed herein, is continued for at least about 1 month, at least about 3 months, at least about 6 months, at least about 9 months, at least about 1 year, at least about 18 months, or at least about 24 months.

The dosing schedule is typically designed to achieve exposures that result in sustained receptor occupancy (RO) based on typical pharmacokinetic properties of an antibody. An exemplary treatment regime entails administration once per week, once every 2 weeks, once every 3 weeks, once every 4 weeks, once a month, once every 3-6 months, or longer. The dosage and scheduling can change during a course of treatment.

For the combination therapy of an anti-TIM3 antibody and an anti-PD-1 antibody, in some embodiments, the anti-TIM3 antibody is administered at a flat dose of any ranges disclosed herein and the anti-PD-1 antibody is administered at a flat dose of any ranges disclosed herein. In some embodiments, the anti-TIM3 antibody is administered at a flat dose of about 3 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 6 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 8 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 24 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 72 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 200 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 480 mg at a dosing interval of 4 weeks and the anti-PD-1 antibody is administered at a flat dose of about 120 mg at a dosing interval of 4 weeks. In some embodiments, the anti-TIM3 antibody is administered at a flat dose of about 800 mg at a dosing interval of 4 weeks and the anti-PD-1 antibody is administered at a flat dose of about 120 mg at a dosing interval of 4 weeks.

In some embodiments, the anti-TIM3 antibody is administered at a flat dose of about 3 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 6 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 8 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 24 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 72 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 200 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 480 mg at a dosing interval of 4 weeks and the anti-PD-1 antibody is administered at a flat dose of about 240 mg at a dosing interval of 4 weeks. In some embodiments, the anti-TIM3 antibody is administered at a flat dose of about 800 mg at a dosing interval of 4 weeks and the anti-PD-1 antibody is administered at a flat dose of about 240 mg at a dosing interval of 4 weeks.

In some embodiments, the anti-TIM3 antibody is administered at a flat dose of about 3 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 6 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 8 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 24 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 72 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 200 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 480 mg at a dosing interval of 4 weeks and the anti-PD-1 antibody is administered at a flat dose of about 480 mg at a dosing interval of 4 weeks. In some embodiments, the anti-TIM3 antibody is administered at a flat dose of about 800 mg at a dosing interval of 4 weeks and the anti-PD-1 antibody is administered at a flat dose of about 480 mg at a dosing interval of 4 weeks.

In some embodiments, the anti-TIM3 antibody is administered at a flat dose of about 3 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 6 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 8 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 24 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 72 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 200 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 480 mg at a dosing interval of 4 weeks and the anti-PD-1 antibody is administered at a flat dose of about 800 mg at a dosing interval of 4 weeks. In some embodiments, the anti-TIM3 antibody is administered at a flat dose of about 800 mg at a dosing interval of 4 weeks and the anti-PD-1 antibody is administered at a flat dose of about 800 mg at a dosing interval of 4 weeks.

In some embodiments, the anti-TIM3 antibody is administered to the subject prior to the administration of the anti-PD-1 antibody. In some embodiments, the anti-TIM3 antibody is administered to the subject after the administration of the anti-PD-1 antibody. In some embodiments, the anti-TIM3 antibody and the anti-PD-1 antibody are administered concurrently in separate compositions. In some embodiments, the anti-TIM3 antibody and the anti-PD-1 antibody are admixed as a single composition for concurrent administration.

In some embodiments, the anti-TIM-3 antibody and a second antibody (e.g., anti-PD1 antibody, anti-PD-L1 antibody, anti-CTLA-4 antibody, anti-LAG-3 antibody, anti-GITR antibody) is formulated in a single composition with a fixed ratio (or dose). For example, the ratio of the two antibodies (e.g., anti-TIM3 antibody in combination with anti-PD1 antibody, anti-PD-L1 antibody, anti-LAG-3 antibody, anti-GITR antibody, or anti-CTLA-4 antibody) is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-TIM3 antibody) to mg second antibody. For example, a 2:1 ratio of an anti-TIM3 antibody and an anti-PD-1 antibody, such as nivolumab, can mean that a vial or an injection can contain about 480 mg of the anti-TIM3 antibody and 240 mg of the anti-PD-1 antibody, or about 2 mg/ml of the anti-TIM3 antibody and 1 mg/ml of the anti-PD-1 antibody. In some embodiments, the composition comprises an anti-TIM3 antibody and an anti-PD1 antibody at a ratio of 1:1, e.g., 480 mg of anti-TIM3 antibody and 480 mg of anti-PD1 antibody or 6 mg/kg of anti-TIM3 antibody and 6 mg/kg anti-PD1 antibody).

In some embodiments, the anti-TIM3 antibody is infused over approximately 30 minutes.

In some embodiments, the PD-1/PD-L1 pathway inhibitor is infused over approximately 30 minutes.

In some embodiments, the PD-1/PD-L1 pathway inhibitor is infused over approximately 30 minutes, after which the anti-TIM3 antibody infusion begins about 30 minutes after completion of the infusion of the PD-1/PD-L1 pathway inhibitor, and the anti-TIM3 antibody is infused over approximately 30 minutes.

### II. Anti-TIM3 Antibodies Useful for the Disclosure

Any anti-TIM3 antibody that is known in the art or disclosed herein can be used in the presently described methods. PCT/US2017/041946 application discloses antibodies that are described herein, and is specifically incorporated by reference herein in its entirety. In some embodiments, the anti-TIM3 antibodies useful for the present disclosure have one or more of the following features:

(1) binding to soluble human TIM3, e.g., with a K_{D} of 10 nM or less (e.g., 0.01 nM to 10 nM), e.g., as measured by BIACORE™;

(2) binding to soluble cynomolgus TIM3, e.g., with a K_{D} of 100 nM or less (e.g., 0.01 nM to 100 nM), e.g., as measured by BIACORE™;

(3) binding to membrane bound human TIM3, e.g., with an EC₅₀ of 1 ug/mL or less (e.g., 0.01 ug/mL to 1 ug/mL), e.g., as measured by flow cytometry;

(4) binding to membrane bound human TIM3, e.g., with a K_{D} of 1 nM or less (e.g., 0.01 nM to 10 nM), e.g., as measured by Scatchard analysis;

(5) binding to membrane bound cynomolgus TIM3, e.g., with an EC₅₀ of 20 ug/mL or less (e.g., 0.01 ug/mL to 20 ug/mL), e.g., as measured by flow cytometry;

(6) binding to membrane bound cynomolgus TIM3, e.g., with a K_{D} of 1 nM or less (e.g., 0.01 nM to 10 nM), e.g., as measured by Scatchard analysis;

(7) inducing or enhancing T cell activation (e.g., by blocking or reducing the inhibitory effects of TIM3), as evidenced by (i) increased IFN-γ production in TIM3-expressing T cells (e.g., Th1 cells or TILs) and/or (ii) enhanced proliferation of TIM3 expressing T cells (e.g., Th1 cells or TILs);

(8) stimulating T cell proliferation in a mixed lymphocyte reaction (MLR) assay;

(9) inhibiting the binding of phosphatidylserine to TIM3, e.g., as measured by PS-hTIM3 “in-tandem” blocking assay;

(10) not internalizing or downregulating cell surface TIM3 when binding to TIM3 on cells;

(11) binding to one of the following regions of human TIM3 extracellular domain (SEQ ID NO: 290): (a) CPVFECG (SEQ ID NO: 296); (b) RIQIPGIMND (SEQ ID NO: 298); (c) CPVFECG and RIQIPGIMND (SEQ ID NOs: 296 and 298, respectively); and (d) WTSRYWLNGDFR (SEQ ID NO: 297);

(12) having reduced binding to human TIM3 in which one or more of amino acids L48, C58, P59, V60, F61, E62, C63, G64, W78, S80, R81, W83, L84, G86, D87, R89, D104, R111, Q113, G116, M118, and D120 (as numbered in SEQ ID NO: 286) is substituted with another amino acid relative to binding to wildtype human TIM3;

(13) competing in either direction or both directions for binding to human TIM3 with an antibody comprising VH and VL domains of any one of 13A3, 3G4, 17C3, 17C8, 9F6, 8B9, 8C4, 14H7, 23B3, TIM3.7, TIM3.8, TIM3.10, TIM3.11, TIM3.12, TIM3.13, TIM3.14, TIM3.15, TIM3.16, TIM3.17, TIM3.18, and TIM3.25;

(14) binding to human TIM3 regions ⁴⁹VPVCWGKGACPVFE⁶² (SEQ ID NO: 367) and ¹¹¹RIQIPGIMNDEKFNLKL¹¹² (SEQ ID NO: 368) as determined by HDX-MS; (15) having the heavy chain and/or light chain variable regions interact with at least 5, 10, 15, 20 or all of the following amino acids of human TIM3: P50, V51, C52, P59, V60, F61, E62, C63, G64, N65, V66, V67, L68, R69, D71, E72, D74, R111, Q113, G116, 1117, M118, D120, and optionally T70 and/or 1112, as determined by X-ray crystallography (numbering per SEQ ID NO: 286); and/or (16) (a) having reduced binding to human TIM3 in which 1, 2, 3, 4, 5, 6, 7, 8 or 9 of amino acids C58, P59, F61, E62, C63, R111, D120, and optionally D104 and Q113 (numbering per SEQ ID NO: 286) are substituted with another amino acid relative to binding to wildtype human TIM3; (b) binding to ⁴⁹VPVCWGKGACPVFE⁶² (SEQ ID NO: 367), ¹¹¹RIQIPGIMNDEKFNLKL¹²⁷ (SEQ ID NO: 368) and ¹¹⁹NDEKFNLKL¹²⁷ (SEQ ID NO: 373), as determined by HDX-MS; and/or (c) competing with or cross-blocking with the binding to human TIM3 of 13A3 or TIM3.18.IgG1.3.

In some embodiments, the anti-TIM3 antibodies revive tumor infiltrating CD8⁺ T cells that co-express PD-1 and TIM3 by combined treatment, hence avoiding depletion of CD8⁺ T cells.

In some embodiments, the anti-TIM3 antibodies comprise:

(a) heavy and light chain variable region sequences comprising SEQ ID NOs: 34 and 60, respectively;

(b) heavy and light chain variable region sequences comprising SEQ ID NOs: 35 and 61, respectively;

(c) heavy and light chain variable region sequences comprising SEQ ID NOs: 36 and 61, respectively;

(d) heavy and light chain variable region sequences comprising SEQ ID NOs: 37 and 60, respectively;

(e) heavy and light chain variable region sequences comprising SEQ ID NOs: 38 and 61, respectively;

(f) heavy and light chain variable region sequences comprising SEQ ID NOs: 38 and 62, respectively;

(g) heavy and light chain variable region sequences comprising SEQ ID NOs: 38 and 63, respectively;

(h) heavy and light chain variable region sequences comprising SEQ ID NOs: 39 and 60, respectively;

(i) heavy and light chain variable region sequences comprising SEQ ID NOs: 40 and 61, respectively;

(j) heavy and light chain variable region sequences comprising SEQ ID NOs: 121 and 63, respectively;

(k) heavy and light chain variable region sequences comprising SEQ ID NOs: 120 and 61, respectively;

(l) heavy and light chain variable region sequences comprising SEQ ID NOs: 112 and 60, respectively;

(m) heavy and light chain variable region sequences comprising SEQ ID NOs: 113 and 60, respectively;

(n) heavy and light chain variable region sequences comprising SEQ ID NOs: 114 and 60, respectively;

(o) heavy and light chain variable region sequences comprising SEQ ID NOs: 115 and 60, respectively;

(p) heavy and light chain variable region sequences comprising SEQ ID NOs: 116 and 60, respectively;

(q) heavy and light chain variable region sequences comprising SEQ ID NOs: 117 and 60, respectively;

(r) heavy and light chain variable region sequences comprising SEQ ID NOs: 118 and 60, respectively;

(s) heavy and light chain variable region sequences comprising SEQ ID NOs: 119 and 60, respectively;

(t) heavy and light chain variable region sequences comprising SEQ ID NOs: 364 and 60, respectively;

(u) heavy and light chain variable region sequences comprising SEQ ID NOs: 410 and 417, respectively;

(v) heavy and light chain variable region sequences comprising SEQ ID NOs: 411 and 60, respectively;

(w) heavy and light chain variable region sequences comprising SEQ ID NOs: 411 and 418, respectively; or

(x) heavy and light chain variable region sequences comprising SEQ ID NOs: 412 and 60, respectively.

In some embodiments, the anti-TIM3 antibodies comprise a heavy chain CDR1, CDR2, and CDR3 and a light chain CDR1, CDR2, and CDR3, wherein:

(a1) the heavy chain CDR1, CDR2, and CDR3 comprises the amino acid sequences of SEQ ID NOs: 41, 46, and 53, respectively, and the light chain CDR1, CDR2, and CDR3 comprises the amino acid sequences of SEQ ID NOs: 64, 66, and 68, respectively;

(a2) the heavy chain CDR1, CDR2, and CDR3 comprises the amino acid sequences of SEQ ID NOs: 41, 122, and 53, respectively, and the light chain CDR1, CDR2, and CDR3 comprises the amino acid sequences of SEQ ID NOs: 64, 66, and 68, respectively;

(a3) the heavy chain CDR1, CDR2, and CDR3 comprises the amino acid sequences of SEQ ID NOs: 41, 123, and 53, respectively, and the light chain CDR1, CDR2, and CDR3 comprises the amino acid sequences of SEQ ID NOs: 64, 66, and 68, respectively;

(a4) the heavy chain CDR1, CDR2, and CDR3 comprises the amino acid sequences of SEQ ID NOs: 41, 124, and 53, respectively, and the light chain CDR1, CDR2, and CDR3 comprises the amino acid sequences of SEQ ID NOs: 64, 66, and 68, respectively;

(a5) the heavy chain CDR1, CDR2, and CDR3 comprises the amino acid sequences of SEQ ID NOs: 41, 46, and 126, respectively, and the light chain CDR1, CDR2, and CDR3 comprises the amino acid sequences of SEQ ID NOs: 64, 66, and 68, respectively;

(a6) the heavy chain CDR1, CDR2, and CDR3 comprises the amino acid sequences of SEQ ID NOs: 41, 46, and 127, respectively, and the light chain CDR1, CDR2, and CDR3 comprises the amino acid sequences of SEQ ID NOs: 64, 66, and 68, respectively;

(a7) the heavy chain CDR1, CDR2, and CDR3 comprises the amino acid sequences of SEQ ID NOs: 41, 46, and 128, respectively, and the light chain CDR1, CDR2, and CDR3 comprises the amino acid sequences of SEQ ID NOs: 64, 66, and 68, respectively;

(a8) the heavy chain CDR1, CDR2, and CDR3 comprises the amino acid sequences of SEQ ID NOs: 41, 46, and 129, respectively, and the light chain CDR1, CDR2, and CDR3 comprises the amino acid sequences of SEQ ID NOs: 64, 66, and 68, respectively;

(a9) the heavy chain CDR1, CDR2, and CDR3 comprises the amino acid sequences of SEQ ID NOs: 41, 122, and 128, respectively, and the light chain CDR1, CDR2, and CDR3 comprises the amino acid sequences of SEQ ID NOs: 64, 66, and 68, respectively;

(a10) the heavy chain CDR1, CDR2, and CDR3 comprises the amino acid sequences of SEQ ID NOs: 41, 122, and 126, respectively, and the light chain CDR1, CDR2, and CDR3 comprises the amino acid sequences of SEQ ID NOs: 64, 66, and 68, respectively;

(b1) the heavy chain CDR1, CDR2, and CDR3 comprises the amino acid sequences of SEQ ID NOs: 42, 47, and 54, respectively, and the light chain CDR1, CDR2, and CDR3 comprises the amino acid sequences of SEQ ID NOs: 64, 66, and 69, respectively;

(b2) the heavy chain CDR1, CDR2, and CDR3 comprises the amino acid sequences of SEQ ID NOs: 42, 125, and 54, respectively, and the light chain CDR1, CDR2, and CDR3 comprises the amino acid sequences of SEQ ID NOs: 64, 66, and 69, respectively;

(c) the heavy chain CDR1, CDR2, and CDR3 comprises the amino acid sequences of SEQ ID NOs: 43, 48, and 55, respectively, and the light chain CDR1, CDR2, and CDR3 comprises the amino acid sequences of SEQ ID NOs: 64, 66, and 69, respectively;

(d) the heavy chain CDR1, CDR2, and CDR3 comprises the amino acid sequences of SEQ ID NOs: 44, 49, and 56, respectively, and the light chain CDR1, CDR2, and CDR3 comprises the amino acid sequences of SEQ ID NOs: 64, 66, and 68, respectively;

(e1) the heavy chain CDR1, CDR2, and CDR3 comprises the amino acid sequences of SEQ ID NOs: 45, 50, and 57, respectively, and the light chain CDR1, CDR2, and CDR3 comprises the amino acid sequences of SEQ ID NOs: 64, 66, and 69, respectively;

(e2) the heavy chain CDR1, CDR2, and CDR3 comprises the amino acid sequences of SEQ ID NOs: 45, 50, and 57, respectively, and the light chain CDR1, CDR2, and CDR3 comprises the amino acid sequences of SEQ ID NOs: 64, 66, and 71, respectively;

(e3) the heavy chain CDR1, CDR2, and CDR3 comprises the amino acid sequences of SEQ ID NOs: 45, 50, and 57, respectively, and the light chain CDR1, CDR2, and CDR3 comprises the amino acid sequences of SEQ ID NOs: 65, 67, and 70, respectively;

(f) the heavy chain CDR1, CDR2, and CDR3 comprises the amino acid sequences of SEQ ID NOs: 45, 51, and 58, respectively, and the light chain CDR1, CDR2, and CDR3 comprises the amino acid sequences of SEQ ID NOs: 64, 66, and 68, respectively;

(g) the heavy chain CDR1, CDR2, and CDR3 comprises the amino acid sequences of SEQ ID NOs: 45, 52, and 59, respectively, and the light chain CDR1, CDR2, and CDR3 comprises the amino acid sequences of SEQ ID NOs: 64, 66, and 69, respectively;

(h) the heavy chain CDR1, CDR2, and CDR3 comprises the amino acid sequences of SEQ ID NOs: 45, 413, and 414, respectively, and the light chain CDR1, CDR2, and CDR3 comprises the amino acid sequences of SEQ ID NOs: 64, 66, and 69, respectively;

(i1) the heavy chain CDR1, CDR2, and CDR3 comprises the amino acid sequences of SEQ ID NOs: 45, 415, and 416, respectively, and the light chain CDR1, CDR2, and CDR3 comprises the amino acid sequences of SEQ ID NOs: 64, 66, and 68, respectively; or

(i2) the heavy chain CDR1, CDR2, and CDR3 comprises the amino acid sequences of SEQ ID NOs: 45, 415, and 416, respectively, and the light chain CDR1, CDR2, and CDR3 comprises the amino acid sequences of SEQ ID NOs: 64, 66, and 419, respectively.

In some embodiments, the anti-TIM3 antibodies comprise:

(a1) heavy and light chain sequences comprising the amino acid sequences of SEQ ID NOs: 301 (or 302) and 29, respectively;

(a2) heavy and light chain sequences comprising the amino acid sequences of SEQ ID NOs: 1 (or 8) and 29, respectively;

(a3) heavy and light chain sequences comprising the amino acid sequences of SEQ ID NOs: 15 (or 22) and 29, respectively;

(a4) heavy and light chain sequences comprising the amino acid sequences of SEQ ID NOs: 303 (or 304) and 29, respectively;

(a5) heavy and light chain sequences comprising the amino acid sequences of SEQ ID NOs: 72 (or 82) and 29, respectively;

(a6) heavy and light chain sequences comprising the amino acid sequences of SEQ ID NOs: 92 (or 102) and 29, respectively;

(a7) heavy and light chain sequences comprising the amino acid sequences of SEQ ID NOs: 305 (or 306) and 29, respectively;

(a8) heavy and light chain sequences comprising the amino acid sequences of SEQ ID NOs: 73 (or 83) and 29, respectively;

(a9) heavy and light chain sequences comprising the amino acid sequences of SEQ ID NOs: 93 (or 103) and 29, respectively;

(a10) heavy and light chain sequences comprising the amino acid sequences of SEQ ID NOs: 307 (or 308) and 29, respectively;

(a11) heavy and light chain sequences comprising the amino acid sequences of SEQ ID NOs: 74 (or 84) and 29, respectively;

(a12) heavy and light chain sequences comprising the amino acid sequences of SEQ ID NOs: 94 (or 104) and 29, respectively;

(a13) heavy and light chain sequences comprising the amino acid sequences of SEQ ID NOs: 309 (or 310) and 29, respectively;

(a14) heavy and light chain sequences comprising the amino acid sequences of SEQ ID NOs: 75 (or 85) and 29, respectively;

(a15) heavy and light chain sequences comprising the amino acid sequences of SEQ ID NOs: 95 (or 105) and 29, respectively;

(a16) heavy and light chain sequences comprising the amino acid sequences of SEQ ID NOs: 311 (or 312) and 29, respectively;

(a17) heavy and light chain sequences comprising the amino acid sequences of SEQ ID NOs: 76 (or 86) and 29, respectively;

(a18) heavy and light chain sequences comprising the amino acid sequences of SEQ ID NOs: 96 (or 106) and 29, respectively;

(a19) heavy and light chain sequences comprising the amino acid sequences of SEQ ID NOs: 313 (or 314) and 29, respectively;

(a20) heavy and light chain sequences comprising the amino acid sequences of SEQ ID NOs: 77 (or 87) and 29, respectively;

(a21) heavy and light chain sequences comprising the amino acid sequences of SEQ ID NOs: 97 (or 107) and 29, respectively;

(a22) heavy and light chain sequences comprising the amino acid sequences of SEQ ID NOs: 315 (or 316) and 29, respectively;

(a23) heavy and light chain sequences comprising the amino acid sequences of SEQ ID NOs: 78 (or 88) and 29, respectively;

(a24) heavy and light chain sequences comprising the amino acid sequences of SEQ ID NOs: 98 (or 108) and 29, respectively;

(a25) heavy and light chain sequences comprising the amino acid sequences of SEQ ID NOs: 317 (or 318) and 29, respectively;

(a26) heavy and light chain sequences comprising the amino acid sequences of SEQ ID NOs: 79 (or 89) and 29, respectively;

(a27) heavy and light chain sequences comprising the amino acid sequences of SEQ ID NOs: 99 (or 109) and 29, respectively;

(a28) heavy and light chain sequences comprising the amino acid sequences of SEQ ID NOs: 319 (or 320) and 29, respectively;

(a29) heavy and light chain sequences comprising the amino acid sequences of SEQ ID NOs: 349 (or 350) and 29, respectively;

(a30) heavy and light chain sequences comprising the amino acid sequences of SEQ ID NOs: 351 (or 352) and 29, respectively;

(a31) heavy and light chain sequences comprising the amino acid sequences of SEQ ID NOs: 353 (or 354) and 29, respectively;

(b1) heavy and light chain sequences comprising the amino acid sequences of SEQ ID NOs: 321 (or 322) and 30, respectively;

(b2) heavy and light chain sequences comprising the amino acid sequences of SEQ ID NOs: 2 (or 9) and 30, respectively;

(b3) heavy and light chain sequences comprising the amino acid sequences of SEQ ID NOs: 16 (or 23) and 30, respectively;

(b4) heavy and light chain sequences comprising S the amino acid sequences of EQ ID NOs: 323 (or 324) and 30, respectively;

(b5) heavy and light chain sequences comprising the amino acid sequences of SEQ ID NOs: 80 (or 90) and 30, respectively;

(b6) heavy and light chain sequences comprising the amino acid sequences of SEQ ID NOs: 100 (or 110) and 30, respectively;

(b7) heavy and light chain sequences comprising the amino acid sequences of SEQ ID NOs: 325 (or 326) and 30, respectively;

(c1) heavy and light chain sequences comprising the amino acid sequences of SEQ ID NOs: 327 (or 328) and 30, respectively;

(c2) heavy and light chain sequences comprising the amino acid sequences of SEQ ID NOs: 3 (or 10) and 30, respectively;

(c3) heavy and light chain sequences comprising the amino acid sequences of SEQ ID NOs: 17 (or 24) and 30, respectively;

(c4) heavy and light chain sequences comprising the amino acid sequences of SEQ ID NOs: 329 (or 330) and 30, respectively;

(d1) heavy and light chain sequences comprising the amino acid sequences of SEQ ID NOs: 331 (or 332) and 29, respectively;

(d2) heavy and light chain sequences comprising the amino acid sequences of SEQ ID NOs: 4 (or 11) and 29, respectively;

(d3) heavy and light chain sequences comprising the amino acid sequences of SEQ ID NOs: 18 (or 25) and 29, respectively;

(d4) heavy and light chain sequences comprising the amino acid sequences of SEQ ID NOs: 333 (or 334) and 29, respectively;

(e1.1) heavy and light chain sequences comprising the amino acid sequences of SEQ ID NOs: 335 (or 336) and 32, respectively;

(e1.2) heavy and light chain sequences comprising the amino acid sequences of SEQ ID NOs: 335 (or 336) and 33, respectively;

(e1.3) heavy and light chain sequences comprising the amino acid sequences of SEQ ID NOs: 335 (or 336) and 31, respectively;

(e2) heavy and light chain sequences comprising the amino acid sequences of SEQ ID NOs: 5 (or 12) and 33, respectively;

(e3) heavy and light chain sequences comprising the amino acid sequences of SEQ ID NOs: 19 (or 26) and 33, respectively;

(e4) heavy and light chain sequences comprising the amino acid sequences of SEQ ID NOs: 337 (or 338) and 33, respectively;

(e5) heavy and light chain sequences comprising the amino acid sequences of SEQ ID NOs: 81 (or 91) and 33, respectively;

(e6) heavy and light chain sequences comprising the amino acid sequences of SEQ ID NOs: 101 (or 111) and 33, respectively;

(e7) heavy and light chain sequences comprising the amino acid sequences of SEQ ID NOs: 339 (or 340) and 33, respectively;

(f1) heavy and light chain sequences comprising the amino acid sequences of SEQ ID NOs: 341 (or 342) and 29, respectively;

(f2) heavy and light chain sequences comprising the amino acid sequences of SEQ ID NOs: 6 (or 13) and 29, respectively;

(f3) heavy and light chain sequences comprising the amino acid sequences of SEQ ID NOs: 20 (or 27) and 29, respectively;

(f4) heavy and light chain sequences comprising the amino acid sequences of SEQ ID NOs: 343 (or 344) and 29, respectively;

(g1) heavy and light chain sequences comprising the amino acid sequences of SEQ ID NOs: 345 (or 346) and 30, respectively;

(g2) heavy and light chain sequences comprising the amino acid sequences of SEQ ID NOs: 7 (or 14) and 30, respectively;

(g3) heavy and light chain sequences comprising the amino acid sequences of SEQ ID NOs: 21 (or 28) and 30, respectively;

(g4) heavy and light chain sequences comprising the amino acid sequences of SEQ ID NOs: 347 (or 348) and 30, respectively;

(h1) heavy and light chain sequences comprising the amino acid sequences of SEQ ID NOs: 386 (or 387) and 408, respectively;

(h2) heavy and light chain sequences comprising the amino acid sequences of SEQ ID NOs: 388 (or 389) and 408, respectively;

(h3) heavy and light chain sequences comprising the amino acid sequences of SEQ ID NOs: 390 (or 391) and 408, respectively;

(h4) heavy and light chain sequences comprising the amino acid sequences of SEQ ID NOs: 392 (or 393) and 408, respectively;

(i1.1) heavy and light chain sequences comprising the amino acid sequences of SEQ ID NOs: 394 (or 395) and 29, respectively;

(i1.2) heavy and light chain sequences comprising the amino acid sequences of SEQ ID NOs: 394 (or 395) and 418, respectively;

(i2) heavy and light chain sequences comprising the amino acid sequences of SEQ ID NOs: 396 (or 397) and 29, respectively;

(i3) heavy and light chain sequences comprising the amino acid sequences of SEQ ID NOs: 398 (or 399) and 29, respectively;

(i4) heavy and light chain sequences comprising the amino acid sequences of SEQ ID NOs: 400 (or 401) and 29, respectively;

(i5) heavy and light chain sequences comprising the amino acid sequences of SEQ ID NOs: 402 (or 403) and 29, respectively;

(i6) heavy and light chain sequences comprising the amino acid sequences of SEQ ID NOs: 404 (or 405) and 29, respectively; or

(i7) heavy and light chain sequences comprising the amino acid sequences of SEQ ID NOs: 406 (or 407) and 29, respectively.

Other anti-TIM3 antibodies that can be used with the present disclosure have been described in, for example, PCT Publication Nos. WO 2003/063792, WO 2010/117057, WO 2011/155607, WO 2011/159877, WO 2013/006490, WO 2015/117002, WO 2016/071448, WO 2016/068802, WO 2016/068803, WO 2016/144803, WO 2016/111947, WO 2017/019897, WO 2017/079112, WO 2017/079115, WO 2017/079116, and WO 2017/055404, each of which is incorporated by reference in its entirety.

The anti-TIM3 antibodies usable in the disclosed methods also include isolated antibodies that bind specifically to human TIM3 and cross-compete for binding to human TIM3 with any of the anti-TIM3 antibodies disclosed herein (e.g., Table 2). In some embodiments, the anti-TIM3 antibodies bind the same epitope as any of the anti-TIM3 antibodies described herein (e.g., Table 2). The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of human TIM3. Cross-competing antibodies can be readily identified based on their ability to cross-compete with any of the anti-TIM3 antibodies disclosed herein in standard binding assays such as Biacore analysis, ELISA assays, or flow cytometry (see, e.g., WO 2013/173223).

In some embodiments, the antibodies that cross-compete for binding to human TIM3 with, or bind to the same epitope region of human TIM3 antibody as, any of the anti-TIM3 antibodies described herein, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-TIM3 antibodies usable in the methods of the present disclosure also include antigen-binding portions of the above antibodies. It has been amply demonstrated that the antigen-binding function of an antibody can be performed by fragments of a full-length antibody.

Anti-TIM3 antibodies suitable for use in the disclosed methods or compositions are antibodies that bind to human TIM3 with high specificity and affinity, block the binding of TIM3, and inhibit the immunosuppressive effect of the TIM3 signaling pathway. In any of the compositions or methods disclosed herein, an anti-TIM3 “antibody” includes an antigen-binding portion or fragment that binds to TIM3 and exhibits the functional properties similar to those of whole antibodies in inhibiting receptor binding and up-regulating the immune system. In some embodiments, the anti-TIM3 antibody or antigen-binding portion thereof cross-competes with any of the above described anti-TIM3 antibodies for binding to human TIM3.

### III. Anti-PD-1 Antibodies Useful for the Disclosure

In some embodiments, the present disclosure comprises administering both an anti-TIM3 antibody and an inhibitor of the PD-1 signaling pathway to a subject in need thereof (e.g., a subject afflicted with a tumor). In some embodiments, the inhibitor of the PD-1 signaling pathway is an anti-PD-1 antibody.

Any anti-PD-1 antibody that is known in the art can be used in the presently described methods. In particular, various human monoclonal antibodies that bind specifically to PD-1 with high affinity have been disclosed in U.S. Pat. No. 8,008,449. Each of the anti-PD-1 humanized antibodies disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (i) inhibits tumor cell growth in vivo. Anti-PD-1 antibodies usable in the present disclosure include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, each of which is incorporated by reference in its entirety.

In some embodiments, the anti-PD-1 antibody useful for the present disclosure is selected from the group consisting of nivolumab (also known as “OPDIVO®”; formerly designated 5C4, BMS-936558, MDX-1106, or ONO-4538), pembrolizumab (Merck, also known as “KEYTRUDA®”, lambrolizumab, and MK-3475. See WO 2008/156712), PDR001 (Novartis; see WO 2015/112900), MEDI-0680 (AstraZeneca; AMP-514; see WO 2012/145493), REGN-2810 (Regeneron; see WO 2015/112800), JS001 (Taizhou Junshi Pharma; see Si-Yang Liu et al., J. Hematol. Oncol. 10:136 (2017)), BGB-A317 (Beigene; see WO 2015/35606 and US 2015/0079109), INCSHRI210 (SHR-1210; Jiangsu Hengrui Medicine; see WO 2015/085847; Si-Yang Liu et al., J Hematol. Oncol. 10:136 (2017)), TSR-042 (ANB011; Tesaro Biopharmaceutical; see WO2014/179664), GLS-010 (WBP3055; Wuxi/Harbin Gloria Pharmaceuticals; see Si-Yang Liu et al., J Hematol. Oncol. 10:136 (2017)), AM-0001 (Armo), STI-1110 (Sorrento Therapeutics; see WO 2014/194302), AGEN2034 (Agenus; see WO 2017/040790), and MGD013 (Macrogenics).

In some embodiments, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In some embodiments, the anti-PD-1 antibody is pembrolizumab. Pembrolizumab is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also worldwideweb.cancer.gov/drugdictionary?cdrid=695789 (last accessed: Dec. 14, 2014). Pembrolizumab has been approved by the FDA for the treatment of relapsed or refractory melanoma, non-small cell lung carcinoma (NSCLC), and head and heck squamous cell carcinoma (HNSCC).

Anti-PD-1 antibodies usable in the disclosed methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In some embodiments, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed methods or compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In some embodiments, the anti-PD-1 antibody or antigen-binding portion thereof cross-competes with nivolumab for binding to human PD-1.

### IV. Anti-PD-L1 Antibodies Useful for the Disclosure

In some embodiments, an anti-PD-1 antibody used according to the methods herein can be replaced with another inhibitor of the PD-1 signaling pathway, for example, an anti-PD-L1 antibody. Any anti-PD-L1 antibody can be used in the methods of the present disclosure. Examples of anti-PD-L1 antibodies useful in the methods of the present disclosure include the antibodies disclosed in U.S. Pat. No. 9,580,507. Each of the anti-PD-L1 human monoclonal antibodies disclosed in U.S. Pat. No. 9,580,507 have been demonstrated to exhibit one or more of the following characteristics: (a) binds to human PD-L1 with a K_{D} of 1×10⁻⁷ M or less, as determined by surface plasmon resonance using a Biacore biosensor system; (b) increases T-cell proliferation in a Mixed Lymphocyte Reaction (MLR) assay; (c) increases interferon-γ production in an MLR assay; (d) increases IL-2 secretion in an MLR assay; (e) stimulates antibody responses; and (f) reverses the effect of T regulatory cells on T cell effector cells and/or dendritic cells. Anti-PD-L1 antibodies usable in the present disclosure include monoclonal antibodies that bind specifically to human PD-L1 and exhibit at least one, in some embodiments, at least five, of the preceding characteristics.

In some embodiments, the anti-PD-L1 antibody is selected from the group consisting of BMS-936559 (formerly 12A4 or MDX-1105; see, e.g., U.S. Pat. No. 7,943,743 and WO 2013/173223), MPDL3280A (also known as RG7446, atezolizumab, and TECENTRIQ®; U.S. Pat. No. 8,217,149; see also, Herbst et al., (2013) J Clin Oncol 31(suppl):3000), durvalumab (IMFINZI®; MEDI-4736; AstraZeneca; see WO 2011/066389), avelumab (Pfizer; MSB-0010718C; BAVENCIO®; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR:Abstract 4606 (April 2016)).

In some embodiments, the anti-PD-L1 monoclonal antibody is selected from the group consisting of 28-8, 28-1, 28-12, 29-8, 5H1, and any combination thereof.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab and/or avelumab. In some embodiments, the anti-PD-L1 antibody binds the same epitope as any of the anti-PD-L1 antibodies described herein, e.g., atezolizumab and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In some embodiments, the antibodies that cross-compete for binding to human PD-L1 with, or bind to the same epitope region of human PD-L1 antibody as, atezolizumab and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In some embodiments, the anti-PD-L1 antibody or antigen-binding portion thereof cross-competes with atezolizumab and/or avelumab for binding to human PD-L1.

### V. Anti-CTLA-4 Antibodies Useful for the Disclosure

In some embodiments, the present disclosure comprises administering the anti-TIM3 antibody in combination with an inhibitor of the cytotoxic T-lymphocyte-associated protein 4 (CTLA-4) signaling pathway. In some embodiments, the anti-TIM3 antibody is administered in combination with both an inhibitor of the CTLA-4 signaling pathway and an inhibitor of the PD-1 signaling pathway (e.g., anti-PD-1 antibody and/or anti-PD-L1 antibody). In some embodiments, the inhibitor of the CTLA-4 signaling pathway is an anti-CTLA-4 antibody.

Any anti-CTLA-4 antibody that bind specifically to human CTLA-4 so as to disrupt the interaction of CTLA-4 with a human B7 receptor can be used in the present disclosure. Because the interaction of CTLA-4 with B7 transduces a signal leading to inactivation of T-cells bearing the CTLA-4 receptor, disruption of the interaction effectively induces, enhances, or prolongs the activation of such T cells, thereby inducing, enhancing, or prolonging an immune response.

Human antibodies (HuMAbs) that bind specifically to CTLA-4 with high affinity have been disclosed in U.S. Pat. Nos. 6,984,720 and 7,605,238. Other anti-CTLA-4 mAbs have been described in, for example, U.S. Pat. Nos. 5,977,318, 6,051,227, 6,682,736, and 7,034,121. The anti-CTLA-4 HuMAbs disclosed in U.S. Pat. Nos. 6,984,720 and 7,605,238 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies usable in the present invention include mAbs that bind specifically to human CTLA-4 and exhibit at least one, at least two, or at least three of the preceding characteristics.

An exemplary clinical anti-CTLA-4 antibody is the human mAb 10D1 (now known as ipilimumab and marketed as YERVOY®) as disclosed in U.S. Pat. No. 6,984,720. In some embodiments, ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

Another anti-CTLA-4 antibody that can be used in the present methods is tremelimumab (also known as CP-675,206). Tremelimumab is a human IgG2 monoclonal anti-CTLA-4 antibody. Tremelimumab is described in WO/2012/122444, U.S. Publ. No. 2012/263677, and WO Publ. No. 2007/113648 A2.

Anti-CTLA-4 antibodies that can be used in the disclosed methods also include isolated antibodies that bind specifically to human CTLA-4 and cross-compete for binding to human CTLA-4 with ipilimumab or tremelimumab or bind to the same epitope region of human CTLA-4 as ipilimumab or tremelimumab. In some embodiments, the antibodies that cross-compete for binding to human CTLA-4 with, or bind to the same epitope region of human CTLA-4 as does ipilimumab or tremelimumab, are antibodies comprising a heavy chain of the human IgG1 isotype. For administration to human subjects, these cross-competing antibodies can be chimeric antibodies, or can be humanized or human antibodies. Usable anti-CTLA-4 antibodies also include antigen-binding portions of the above antibodies such as Fab, F(ab′)₂, Fd or Fv fragments.

### VI. Standard-of-Care Therapies

The present disclosure also comprises administering an anti-TIM3 antibody to a subject in need thereof in combination with or following a standard-of-care therapy for one or more of the cancers disclosed herein. In some embodiments, the anti-TIM3 antibody and the standard-of-care therapy are administered with an additional immunotherapeutic antibody (e.g., anti-PD-1 antibody and/or anti-PD-L1 antibody).

In some embodiments, the standard-of-care therapy can be performed on the subject any time before, during, or after the administration of the anti-TIM3 antibody. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014, available at: worldwideweb.nccn.org/professionals/physician_gls/fguidelines.asp, last accessed Jan. 2, 2018). Non-limiting examples of standard-of-care therapies for the different cancers are provided below.

In some embodiments, the standard-of-care therapy comprises surgery, radiation therapy (RT) (i.e., use of high-energy x-rays to destroy cancer cells), chemotherapy, targeted therapy (i.e., use of drugs or other substances that interfere with specific molecules involved in the growth, progression, and/or spread of tumors), or any combinations thereof. In some embodiments, surgery comprises surgical resection (i.e., physical removal of the tumor and some surrounding healthy tissue). In some embodiments, radiation therapy comprises external-beam radiation therapy (e.g., stereotactic radiation therapy), intraoperative radiation therapy, brachytherapy, or any combinations thereof. In some embodiments, the radiation therapy is administered prior to surgery (e.g., neoadjuvant therapy) or after surgery to destroy any remaining cancer cells.

In some embodiments, the chemotherapy comprises platinum agents (e.g., cisplatin (PLATINOL®), carboplatin (PARAPLATIN®)), taxane agents (e.g., paclitaxel (TAXOL®), albumin-bound paclitaxel, docetaxel (TAXOTERE®)), vinorelbine (NAVELBINE®), vinblastine (VELBAN®), etoposide (TOPOSAR®, EPOSIN®, ETOPOPHOS®, VEPESID®), pemetrexed (ALIMTA®), or gemcitabine (GEMZAR®). In some embodiments, the standard-of-care therapy for TNBC comprises anthracycline or anthracycline/taxane-based chemotherapy (e.g., doxorubicin (ADRIAMYCIN®, DOXIL®, LIPODOX®) and cyclophosphamide (CYTOXAN®). In some embodiments, chemotherapy comprises capecitabine (XELODA®), fluorouracil (5-FU, ADRUCIL®), irinotecan (CAMPTOSAR®), Oxaliplatin (ELOXATIN®), Trifluridine/tipiracil (TAS-102, LONSURF®), or any combinations thereof. Common treatment regimens that include the above chemotherapeutic drugs include: 5-FU alone; 5-FU with leucovorin (WELLCOVORIN®), a vitamin that improves the effectiveness of 5-FU; Capecitabine (oral form of 5-FU); FOLFOX (5-FU with leucovorin and oxaliplatin); FOLFIRI (5-FU with leucovorin and irinotecan); irinotecan alone; XELIRI/CAPIRI (capectiabine with irinotecan); and XELOX/CAPEOX (capecitabine with oxaliplatin).

In some embodiments, the targeted therapy comprises anti-angiogenic agents (e.g., sorafenib (NEXAVAR), sunitinib (SUTENT®), pazopanib (VOTRIENT®), axitinib (INLYTA®), and tivozanib) or mammalian target of rapamycin (mTOR) inhibitors (e.g., everolimus (AFINITOR®) and temsirolimus (TORISEL®)). In some embodiments, the targeted therapy comprises bevacizumab (AVASTIN®), erlotinib (TARCEVA®), crizotinib (XALKORI®), or cetuximab (ERBITUX®).

### V. Cancers

Inhibition of TIM3 by anti-TIM3 antibodies can enhance the immune response to cancerous cells in a patient having cancer. Provided herein are methods for treating a subject afflicted with a tumor, comprising administering to the subject an anti-TIM3 antibody described herein, such that the subject is treated, e.g., such that growth of cancerous tumors is inhibited or reduced and/or that the tumors regress and/or that prolonged survival is achieved. In some embodiments, the anti-TIM3 antibody can be used alone to inhibit the growth of cancerous tumors. In some embodiments, the anti-TIM3 antibody can be used in conjunction with another agent, e.g., an inhibitor of the PD-1 signaling pathway (e.g., an anti-PD-1 antibody and/or an anti-PD-L1 antibody).

Tumors whose growth can be inhibited using the methods of the present disclosure include tumors typically responsive to immunotherapy and those that are not typically responsive to immunotherapy. Tumors that can be treated also include TIM3 positive tumors. In some embodiments, the tumors are also positive for PD-L1 and/or PD-L2 expression. In some embodiments, the tumor is derived from a cancer selected from the group consisting of a squamous cell carcinoma, small-cell lung cancer, non-small cell lung cancer, squamous non-small cell lung cancer (NSCLC) (e.g., stage IIIB, stage IV, recurrent, or refractory to platinum doublet-based chemotherapy), nonsquamous NSCLC, glioma, gastrointestinal cancer, renal cancer (e.g., clear cell carcinoma), ovarian cancer, liver cancer (e.g., hepatocellular carcinoma), colorectal cancer (CRC) (e.g., refractory to at least one treatment with a standard systemic therapy for metastatic and/or unresectable disease, including Oxaliplatin and Irinotecan), endometrial cancer, kidney cancer (e.g., renal cell carcinoma (RCC), e.g., advanced or metastatic with a clear cell component, refractory to a single treatment of an anti-angiogenic therapy regimen (including but not limited to sunitinib, sorafenib, pazopanib, axitnib, tivozanib, and bevacizumab)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer (e.g., triple negative breast cancer (TNBC), e.g., recurrent or metastatic TNBC, refractory to at least one standard chemotherapy regimen containing anthracycline and taxane), colon carcinoma, and head and neck cancer (or carcinoma) (e.g., squamous cell carcinoma of the head and neck (SCCHN), e.g., refractory to a platinum-containing regimen), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combination thereof. In some embodiments, the cancer is an advanced, recurring, metastatic, and/or refractory cancer. In some embodiments, the cancers that can be treated with the present disclosure are refractory to prior anti-PD-L1 therapy.

In some embodiments, a method of treating cancer in a subject comprises first determining whether the subject is TIM3 positive, e.g., has tumor cells or TILs that express TIM3 or soluble TIM3, e.g., in the blood, and if the subject has TIM3 positive cancer or TIL cells or soluble TIM3, then administering to the subject an anti-TIM3 antibody, e.g., described herein. A method of treating a subject having cancer with an anti-TIM3 antibody can comprise administering to a subject who has cancer cells or TIL cells that express TIM3 or soluble TIM3, a therapeutically effective amount of a TIM3 antibody. Also provided herein are methods for predicting whether a subject will respond to treatment with an anti-TIM3 antibody, wherein the methods comprise determining the level of TIM3 in cancer or TIL cells of the patient or the amount of soluble TIM3, e.g., in the blood, and if cancer or TIL cells of the subject are TIM3 positive or if the subject has soluble TIM3, then the subject is likely to respond to a treatment with a TIM3 antibody.

In some embodiments, a method of treating cancer in a subject comprises first determining whether the subject is PD-L1 or PD-1 positive, e.g., has tumor cells or TILs that express PD-L1 or PD-1, and if the subject has PD-L1 or PD-1 positive cancer or TIL cells, then administering to the subject an anti-TIM3 antibody (and optionally a PD-1 or PD-L1 antagonist), e.g., described herein. A method of treating a subject having cancer with an anti-TIM3 antibody (and optionally in combination with a PD-1 or PD-L1 antagonist) can comprise administering to a subject who has cancer cells or TIL cells that express PD-L1 or PD-1, a therapeutically effective amount of a TIM3 antibody (and optionally a PD-1 or PD-L1 antagonist).

VII. TIM3, PD-L1, and/or PD-L2 Expression Status

The TIM3, PD-L1, and/or PD-L2 expression status of a tumor in a subject can be measured prior to administering any composition or utilizing any method disclosed herein. TIM3, PD-L1, and/or PD-L2 expression can be determined by any methods known in the art.

In order to assess the TIM3, PD-L1, and/or PD-L2 expression, in some embodiments, a test sample (e.g., tissue or blood) can be obtained from the patient who is in need of the therapy. In some embodiments, the assessment of TIM3, PD-L1, and/or PD-L2 expression can be achieved without obtaining a test sample. In some embodiments, selecting a suitable patient includes (i) optionally providing a test sample obtained from a patient with a cancer, the test sample comprising tumor cells and/or tumor-infiltrating inflammatory cells; and (ii) assessing the proportion of cells in the test sample that express TIM3, PD-L1, and/or PD-L2 on the surface of the cells based on an assessment that the proportion of cells in the test sample that express TIM3, PD-L1, and/or PD-L2 on the cell surface is higher than a predetermined threshold level.

In any of the methods comprising the measurement of TIM3, PD-L1, and/or PD-L2 expression in a test sample, however, it should be understood that the step comprising the provision of a test sample obtained from a patient is an optional step. It should also be understood that, in some embodiments, the “measuring” or “assessing” step to identify, or determine the number or proportion of, cells in the test sample that express TIM3, PD-L1, and/or PD-L2 on the cell surface is performed by a transformative method of assaying for TIM3, PD-L1, and/or PD-L2 expression, for example by performing a reverse transcriptase-polymerase chain reaction (RT-PCR) assay or an IHC assay. In some embodiments, no transformative step is involved and TIM3, PD-L1, and/or PD-L2 expression is assessed by, for example, reviewing a report of test results from a laboratory. In some embodiments, the steps of the methods up to, and including, assessing TIM3, PD-L1, and/or PD-L2 expression provides an intermediate result that may be provided to a physician or other healthcare provider for use in selecting a suitable candidate for the methods of the present disclosure. In some embodiments, the steps that provide the intermediate result is performed by a medical practitioner or someone acting under the direction of a medical practitioner. In some embodiments, these steps are performed by an independent laboratory or by an independent person such as a laboratory technician.

In some embodiments, the proportion of cells that express TIM3, PD-L1, and/or PD-L2 or the amount of soluble TIM3 is assessed by performing an assay to determine the presence of TIM3, PD-L1, and/or PD-L2 RNA. In some embodiments, the presence of TIM3, PD-L1, and/or PD-L2 RNA is determined by RT-PCR, in situ hybridization or RNase protection. In some embodiments, the proportion of cells that express TIM3, PD-L1, and/or PD-L2 or the amount of soluble TIM3 is assessed by performing an assay to determine the presence of TIM3, PD-L1, and/or PD-L2 polypeptide. In some embodiments, the presence of TIM3, PD-L1, and/or PD-L2 polypeptide is determined by immunohistochemistry (IHC), enzyme-linked immunosorbent assay (ELISA), in vivo imaging, or flow cytometry. In some embodiments, TIM3, PD-L1, and/or PD-L2 expression is assayed by IHC. In some embodiments of all of these methods, cell surface expression of TIM3, PD-L1, and/or PD-L2 is assayed using, e.g., IHC or in vivo imaging. Chen et al., (2013) Clin Cancer Res 19(13): 3462-3473.

In some embodiments, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100% of the tumor cells and/or tumor-infiltrating inflammatory cells express TIM3, PD-L1, and/or PD-L2.

### VIII. Pharmaceutical Compositions

Therapeutic agents of the present disclosure can be constituted in a composition, e.g., a pharmaceutical composition containing an antibody and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In some embodiments, the carrier for a composition containing an antibody is suitable for intravenous, intramuscular, subcutaneous, parenteral, spinal, or epidermal administration (e.g., by injection or infusion). A pharmaceutical composition of the disclosure can include one or more pharmaceutically acceptable salts, anti-oxidants, aqueous and non-aqueous carriers, and/or adjuvants such as preservatives, wetting agents, emulsifying agents, and dispersing agents.

### IX. Kits

Also within the scope of the present disclosure are kits comprising an anti-TIM3 antibody, alone or in combination with an inhibitor of the PD-1 signaling pathway (e.g., an anti-PD-1 antibody) for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a flat dosage of an anti-TIM3 antibody; (b) optionally, a flat dosage of an anti-PD-1 antibody; and (c) instructions for using the enclosed antibody or antibodies in any of the combination therapy methods disclosed herein. In some embodiments, the anti-TIM3 antibody and the anti-PD-1 antibody can be co-packaged in unit dosage form. In some embodiments for treating human patients, the kit comprises an anti-TIM3 antibody disclosed herein. In some embodiments, the kit comprises an anti-PD-1 antibody disclosed herein, e.g., nivolumab, pembrolizumab, MEDI0680 (formerly AMP-514), AMP-224, or BGB-A317.

TABLE 2

| SEQ | | |
|---|---|---|
| ID | Description | Sequences |
| 1 | TIM3.5 (13A3) | QLQLQESGPGLVKPSETLSLTCTVSGGSISSRSYYWGWIRQPPGKGLEWIGSTYYSGET |
| | IgG1.1f Heavy | YYNPSLKSRVTISVDTSKNQFSLKLSSVTAADTAVYYCATGGPYGDYAHWFDPWGQGTL |
| | Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 2 | 8B9 IgG1.1f | QVQLQESGPGLVKPSETLSLTCTVSGGSISRHYWNWIRQPPGKGLEWIGYIHYSGSTNY |
| | Heavy Chain | NSSLKSRVTISVDTSKNQFSLKLSSVTAADTAVYYCARDTGYYGMDIWGQGTTVTVSSA |
| | | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPSSIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPGK* |
| 3 | TIM3.6 (8C4) | QVQLQESGPGLVKPSETLSLTCTVSGGSISRYYWSWIRQPPGKGLEWIGYIHYTGSTNY |
| | IgG1.1f Heavy | NPSLKSRVTISVDTSKNQFSLKLSSVTAADTAVYYCATDTGYYGMDVWGQGTTVTVSSA |
| | Chain | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPSSIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPGK* |
| 4 | TIM3.2 (17C3) | QVQLVQSGAEVKKPGASVKVSCKASGYTFTSYYMHWVRQAPGQGLEWMGIINPRGDSII |
| | IgG1.1f Heavy | YAQKFQGRVTMTRDTSTSTVYMELSSLRSEDTAVYYCARDFYGSGNYYYGMDVWGQGTT |
| | Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 5 | 9F6 IgG1.1f | QVQLVESGGGLVKPGGSLRLSCAASGFTESDYYMSWIRQAPGKGLEWVSFISGGGSTIY |
| | Heavy Chain | YADSVKGRFTISRDNAKNSLFLQMNSLRVEDTAVYYCARDGYSSGWYYYGMDVWGQGTA |
| | | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 6 | TIM3.4 (3G4) | QVQLVESGGGLVKPGGSLRLSCAASGFTESDYYMSWIRQAPGKGLEWVSFISTSGSITY |
| | IgG1.1f Heavy | YADSVKGRETISRDNAKNSLYLQMNSLRAEDTAVYYCAREGYSSSWSYYYGMDVWGQGT |
| | Chain | TVTVSSASTKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPKSCDKTHTCPPC |
| | | PAPEAEGAPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPSSIEKTISKAKGQPREPQ |
| | | VYTLPPSREEMTKNQVSLTCLVKGFYPSDIAVEWESNGQPENNYKTTPPVLDSDGSFEL |
| | | YSKLTVDKSRWQQGNVFSCSVMHEALHNHYTQKSLSLSPGK* |
| 7 | TIM3.9 (17C8) | QVQLVESGGGLVKPGGSLRLSCAASGFTESDYYMSWIRQAPGKGLEWVSFISSSGSITY |
| | IgG1.1f Heavy | YADSVKGRETISRDNAKNSLYLQMNSLRAEDTAVYYCARDGYSSGWEYYGMDVWGQGTT |
| | Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 8 | TIM3.5 (13A3) | QLQLQESGPGLVKPSETLSLTCTVSGGSISSRSYYWGWIRQPPGKGLEWIGSTYYSGET |
| | IgG1.1f Heavy | YYNPSLKSRVTISVDTSKNQFSLKLSSVTAADTAVYYCATGGPYGDYAHWFDPWGQGTL |
| | Chain (without | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 9 | 8B9 IgG1.1f | QVQLQESGPGLVKPSETLSLTCTVSGGSISRHYWNWIRQPPGKGLEWIGYIHYSGSTNY |
| | Heavy Chain | NSSLKSRVTISVDTSKNQFSLKLSSVTAADTAVYYCARDTGYYGMDIWGQGTTVTVSSA |
| | (without C- | STKGPSVFPLAPSSKSTSGGTAALGCLVKDYFPEPVTVSWNSGALTSGVHTFPAVLQSS |
| | terminal K) | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPSSIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPG* |
| 10 | IgG1.1f Heavy | QVQLQESGPGLVKPSETLSLTCTVSGGSISRYYWSWIRQPPGKGLEWIGYIHYTGSTNY |
| | TIM3.6 (8C4) | NPSLKSRVTISVDTSKNQFSLKLSSVTAADTAVYYCATDTGYYGMDVWGQGTTVTVSSA |
| | Chain (without | STKGPSVFPLAPSSKSTSGGTAALGCLVKDYFPEPVTVSWNSGALTSGVHTFPAVLQSS |
| | C-terminal K) | GLYSLSSVVTVPSSSLGTQTYICNVNHKPSNTKVDKRVEPKSCDKTHTCPPCPAPEAEG |
| | | APSVFLFEPPKPKDTLMISRTPEVTCVVVDVSHEDPEVKFNWYVDGVEVHNAKTKPREEQ |
| | | YNSTYRVVSVLTVLHQDWLNGKEYKCKVSNKALPSSIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPG* |
| 11 | TIM3.2 (17C3) | QVQLVQSGAEVKKPGASVKVSCKASGYTFTSYYMHWVRQAPGQGLEWMGIINPRGDSII |
| | IgG1.1f Heavy | YAQKFQGRVTMTRDTSTSTVYMELSSLRSEDTAVYYCARDFYGSGNYYYGMDVWGQGTT |
| | Chain (without | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 12 | 9F6 IgG1.1f | QVQLVESGGGLVKPGGSLRLSCAASGFTESDYYMSWIRQAPGKGLEWVSFISGGGSTIY |
| | Heavy Chain | YADSVKGRETISRDNAKNSLFLQMNSLRVEDTAVYYCARDGYSSGWYYYGMDVWGQGTA |
| | (without C- | VTVSSASTKGPSVFPLAPSSKSTSGGTAALGCLVKDYFPEPVTVSWNSGALTSGVHTFP |
| | terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 13 | TIM3.4 (3G4) | QVQLVESGGGLVKPGGSLRLSCAASGFTESDYYMSWIRQAPGKGLEWVSFISTSGSITY |
| | IgG1.1f Heavy | YADSVKGRETISRDNAKNSLYLQMNSLRAEDTAVYYCAREGYSSSWSYYYGMDVWGQGT |
| | Chain (without | TVTVSSASTKGPSVFPLAPSSKSTSGGTAALGCLVKDYFPEPVTVSWNSGALTSGVHTF |
| | C-terminal K) | PAVLQSSGLYSLSSVVTVPSSSLGTQTYICNVNHKPSNTKVDKRVEPKSCDKTHTCPPC |
| | | PAPEAEGAPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPSSIEKTISKAKGQPREPQ |
| | | VYTLPPSREEMTKNQVSLTCLVKGFYPSDIAVEWESNGQPENNYKTTPPVLDSDGSFEL |
| | | YSKLTVDKSRWQQGNVFSCSVMHEALHNHYTQKSLSLSPG* |
| 14 | TIM3.9 (17C8) | QVQLVESGGGLVKPGGSLRLSCAASGFTESDYYMSWIRQAPGKGLEWVSFISSSGSITY |
| | IgG1.1f Heavy | YADSVKGRETISRDNAKNSLYLQMNSLRAEDTAVYYCARDGYSSGWEYYGMDVWGQGTT |
| | Chain (without | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 15 | TIM3.5 (13A3) | QLQLQESGPGLVKPSETLSLTCTVSGGSISSRSYYWGWIRQPPGKGLEWIGSTYYSGET |
| | IgG1.3f Heavy | YYNPSLKSRVTISVDTSKNQFSLKLSSVTAADTAVYYCATGGPYGDYAHWFDPWGQGTL |
| | Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 16 | 8B9 IgG1.3f | QVQLQESGPGLVKPSETLSLTCTVSGGSISRHYWNWIRQPPGKGLEWIGYIHYSGSTNY |
| | Heavy Chain | NSSLKSRVTISVDTSKNQFSLKLSSVTAADTAVYYCARDTGYYGMDIWGQGTTVTVSSA |
| | | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPGK* |
| 17 | TIM3.6 (8C4) | QVQLQESGPGLVKPSETLSLTCTVSGGSISRYYWSWIRQPPGKGLEWIGYIHYTGSTNY |
| | IgG1.3f Heavy | NPSLKSRVTISVDTSKNQFSLKLSSVTAADTAVYYCATDTGYYGMDVWGQGTTVTVSSA |
| | Chain | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPGK* |
| 18 | TIM3.2 (17C3) | QVQLVQSGAEVKKPGASVKVSCKASGYTFTSYYMHWVRQAPGQGLEWMGIINPRGDSII |
| | IgG1.3f Heavy | YAQKFQGRVTMTRDTSTSTVYMELSSLRSEDTAVYYCARDFYGSGNYYYGMDVWGQGTT |
| | Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 19 | 9F6 IgG1.3f | QVQLVESGGGLVKPGGSLRLSCAASGFTESDYYMSWIRQAPGKGLEWVSFISGGGSTIY |
| | Heavy Chain | YADSVKGRFTISRDNAKNSLFLQMNSLRVEDTAVYYCARDGYSSGWYYYGMDVWGQGTA |
| | | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 20 | TIM3.4 (3G4) | QVQLVESGGGLVKPGGSLRLSCAASGFTESDYYMSWIRQAPGKGLEWVSFISTSGSITY |
| | IgG1.3f Heavy | YADSVKGRETISRDNAKNSLYLQMNSLRAEDTAVYYCAREGYSSSWSYYYGMDVWGQGT |
| | Chain | TVTVSSASTKGPSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPKSCDKTHTCPPC |
| | | PAPEAEGAPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKTTPPVLDSDGSFEL |
| | | YSKLTVDKSRWQQGNVFSCSVMHEALHNHYTQKSLSLSPGK* |
| 21 | TIM3.9 (17C8) | QVQLVESGGGLVKPGGSLRLSCAASGFTESDYYMSWIRQAPGKGLEWVSFISSSGSITY |
| | IgG1.3f Heavy | YADSVKGRETISRDNAKNSLYLQMNSLRAEDTAVYYCARDGYSSGWEYYGMDVWGQGTT |
| | Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 22 | TIM3.5 (13A3) | QLQLQESGPGLVKPSETLSLTCTVSGGSISSRSYYWGWIRQPPGKGLEWIGSTYYSGET |
| | IgG1.3f Heavy | YYNPSLKSRVTISVDTSKNQFSLKLSSVTAADTAVYYCATGGPYGDYAHWFDPWGQGTL |
| | Chain (no C- | VTVSSASTKGPSVFPLAPSSKSTSGGTAALGCLVKDYFPEPVTVSWNSGALTSGVHTFP |
| | terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 23 | 8B9 IgG1.3f | QVQLQESGPGLVKPSETLSLTCTVSGGSISRHYWNWIRQPPGKGLEWIGYIHYSGSTNY |
| | Heavy Chain (no | NSSLKSRVTISVDTSKNQFSLKLSSVTAADTAVYYCARDTGYYGMDIWGQGTTVTVSSA |
| | C-terminal K) | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPG* |
| 24 | TIM3.6 (8C4) | QVQLQESGPGLVKPSETLSLTCTVSGGSISRYYWSWIRQPPGKGLEWIGYIHYTGSTNY |
| | IgG1.3f Heavy | NPSLKSRVTISVDTSKNQFSLKLSSVTAADTAVYYCATDTGYYGMDVWGQGTTVTVSSA |
| | Chain (no C- | STKGPSVFPLAPSSKSTSGGTAALGCLVKDYFPEPVTVSWNSGALTSGVHTFPAVLQSS |
| | terminal K) | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPG* |
| 25 | TIM3.2 (17C3) | QVQLVQSGAEVKKPGASVKVSCKASGYTFTSYYMHWVRQAPGQGLEWMGIINPRGDSII |
| | IgG1.3f Heavy | YAQKFQGRVTMTRDTSTSTVYMELSSLRSEDTAVYYCARDFYGSGNYYYGMDVWGQGTT |
| | Chain (no C- | VTVSSASTKGPSVFPLAPSSKSTSGGTAALGCLVKDYFPEPVTVSWNSGALTSGVHTFP |
| | terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 26 | 9F6 IgG1.3f | QVQLVESGGGLVKPGGSLRLSCAASGFTESDYYMSWIRQAPGKGLEWVSFISGGGSTIY |
| | Heavy Chain (no | YADSVKGRFTISRDNAKNSLFLQMNSLRVEDTAVYYCARDGYSSGWYYYGMDVWGQGTA |
| | C-terminal K) | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 27 | TIM3.4 (3G4) | QVQLVESGGGLVKPGGSLRLSCAASGFTESDYYMSWIRQAPGKGLEWVSFISTSGSITY |
| | IgG1.3f Heavy | YADSVKGRETISRDNAKNSLYLQMNSLRAEDTAVYYCAREGYSSSWSYYYGMDVWGQGT |
| | Chain (no C- | TVTVSSASTKGPSVFPLAPSSKSTSGGTAALGCLVKDYFPEPVTVSWNSGALTSGVHTF |
| | terminal K) | PAVLQSSGLYSLSSVVTVPSSSLGTQTYICNVNHKPSNTKVDKRVEPKSCDKTHTCPPC |
| | | PAPEAEGAPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKTTPPVLDSDGSFEL |
| | | YSKLTVDKSRWQQGNVFSCSVMHEALHNHYTQKSLSLSPG* |
| 28 | TIM3.9 (17C8) | QVQLVESGGGLVKPGGSLRLSCAASGFTESDYYMSWIRQAPGKGLEWVSFISSSGSITY |
| | IgG1.3f Heavy | YADSVKGRETISRDNAKNSLYLQMNSLRAEDTAVYYCARDGYSSGWEYYGMDVWGQGTT |
| | Chain (no C- | VTVSSASTKGPSVFPLAPSSKSTSGGTAALGCLVKDYFPEPVTVSWNSGALTSGVHTFP |
| | terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 72 | TIM3.10 (13A3) | QLQLQESGPGLVKPSETLSLTCTVSGGSISSRSYYWGWIRQPPGKGLEWIGSTYYSGET |
| | IgG1.1f (N60Q) | YYQPSLKSRVTISVDTSKNQFSLKLSSVTAADTAVYYCATGGPYGDYAHWFDPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 73 | TIM3.11 (13A3) | QLQLQESGPGLVKPSETLSLTCTVSGGSISSRSYYWGWIRQPPGKGLEWIGSTYYSGET |
| | IgG1.1f (N60S) | YYSPSLKSRVTISVDTSKNQFSLKLSSVTAADTAVYYCATGGPYGDYAHWFDPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 74 | TIM3.12 (13A3) | QLQLQESGPGLVKPSETLSLTCTVSGGSISSRSYYWGWIRQPPGKGLEWIGSTYYSGET |
| | IgG1.1f (N60A) | YYAPSLKSRVTISVDTSKNQFSLKLSSVTAADTAVYYCATGGPYGDYAHWFDPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 75 | TIM3.13 (13A3) | QLQLQESGPGLVKPSETLSLTCTVSGGSISSRSYYWGWIRQPPGKGLEWIGSTYYSGET |
| | IgG1.1f (D101E) | YYNPSLKSRVTISVDTSKNQFSLKLSSVTAADTAVYYCATGGPYGDYAHWEEPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 76 | TIM3.14 (13A3) | QLQLQESGPGLVKPSETLSLTCTVSGGSISSRSYYWGWIRQPPGKGLEWIGSTYYSGET |
| | IgG1.1f (P102V) | YYNPSLKSRVTISVDTSKNQFSLKLSSVTAADTAVYYCATGGPYGDYAHWEDVWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 77 | TIM3.15 (13A3) | QLQLQESGPGLVKPSETLSLTCTVSGGSISSRSYYWGWIRQPPGKGLEWIGSTYYSGET |
| | IgG1.1f (P102Y) | YYNPSLKSRVTISVDTSKNQFSLKLSSVTAADTAVYYCATGGPYGDYAHWFDYWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 78 | TIM3.16 (13A3) | QLQLQESGPGLVKPSETLSLTCTVSGGSISSRSYYWGWIRQPPGKGLEWIGSTYYSGET |
| | IgG1.1f (P102L) | YYNPSLKSRVTISVDTSKNQFSLKLSSVTAADTAVYYCATGGPYGDYAHWFDLWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 79 | TIM3.17 (13A3) | QLQLQESGPGLVKPSETLSLTCTVSGGSISSRSYYWGWIRQPPGKGLEWIGSTYYSGET |
| | IgG1.1f | YYQPSLKSRVTISVDTSKNQFSLKLSSVTAADTAVYYCATGGPYGDYAHWFDYWGQGTL |
| | (N60Q/P102Y) | VTVSSASTKGPSVFPLAPSSKSTSGGTAALGCLVKDYFPEPVTVSWNSGALTSGVHTFP |
| | Heavy Chain | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 349 | TIM3.18 (13A3) | QLQLQESGPGLVKPSETLSLTCTVSGGSISSRSYYWGWIRQPPGKGLEWIGSTYYSGET |
| | IgG1.1f | YYQPSLKSRVTISVDTSKNQFSLKLSSVTAADTAVYYCATGGPYGDYAHWEEPWGQGTL |
| | (N60Q/D101E) | VTVSSASTKGPSVFPLAPSSKSTSGGTAALGCLVKDYFPEPVTVSWNSGALTSGVHTFP |
| | Heavy Chain | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 80 | TIM3.8 (8B9) | QVQLQESGPGLVKPSETLSLTCTVSGGSISRHYWNWIRQPPGKGLEWIGYIHYSGSTNY |
| | IgG1.1f (S61P) | NPSLKSRVTISVDTSKNQFSLKLSSVTAADTAVYYCARDTGYYGMDIWGQGTTVTVSSA |
| | Heavy Chain | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPSSIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPGK* |
| 81 | TIM3.7 (9F6) | QVQLVESGGGLVKPGGSLRLSCAASGFTESDYYMSWIRQAPGKGLEWVSFISGGGSTIY |
| | IgG1.1f (A108T) | YADSVKGRFTISRDNAKNSLFLQMNSLRVEDTAVYYCARDGYSSGWYYYGMDVWGQGTT |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 388 | TIM3.24/14H7 | QVHLVESGGGLVKPGGSLRLSCTAFSDYYMSWIRQAPGKGLEWLSYISNSGSITYYADS |
| | IgG1.1f Heavy | VKGRETISRDNAKNSVYLQMNSLRAEDTAVYYCARGRIGFEDYWGPGTLVTVSSASTKG |
| | Chain | PSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKRVEPKSCDKTHTCPPCPAPEAEGAPSV |
| | | FLFPPKPKDTLMISRTPEVTCVVVDVSHEDPEVKFNWYVDGVEVHNAKTKPREEQYNST |
| | | YRVVSVLTVLHQDWLNGKEYKCKVSNKALPSSIEKTISKAKGQPREPQVYTLPPSREEM |
| | | TKNQVSLTCLVKGFYPSDIAVEWESNGQPENNYKTTPPVLDSDGSFELYSKLTVDKSRW |
| | | QQGNVFSCSVMHEALHNHYTQKSLSLSPGK* |
| 396 | 23B3 IgG1.1f | QVQLVGSGGGLVKPGGSLRLSCAASGFTESDYYMSWIRQAPGKGLEWVSFISGSGSITY |
| | Heavy Chain | YADSVKGRETISRDNAKNSLDLQMNSLRAEDTAVYYCARDGMVRGMNFYGMDVWGQGTT |
| | | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 402 | TIM3.25 (23B3) | QVQLVESGGGLVKPGGSLRLSCAASGFTESDYYMSWIRQAPGKGLEWVSFISGSGSITY |
| | IgG1.1f (G6E, | YADSVKGRETISRDNAKNSLYLQMNSLRAEDTAVYYCARDGMVRGMNFYGMDVWGQGTT |
| | D79Y) Heavy | VTVSSASTKGPSVFPLAPSSKSTSGGTAALGCLVKDYFPEPVTVSWNSGALTSGVHTFP |
| | Chain | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 82 | TIM3.10 (13A3) | QLQLQESGPGLVKPSETLSLTCTVSGGSISSRSYYWGWIRQPPGKGLEWIGSTYYSGET |
| | IgG1.1f (N60Q) | YYQPSLKSRVTISVDTSKNQFSLKLSSVTAADTAVYYCATGGPYGDYAHWFDPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 83 | TIM3.11 (13A3) | QLQLQESGPGLVKPSETLSLTCTVSGGSISSRSYYWGWIRQPPGKGLEWIGSTYYSGET |
| | IgG1.1f (N60S) | YYSPSLKSRVTISVDTSKNQFSLKLSSVTAADTAVYYCATGGPYGDYAHWFDPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 84 | TIM3.12 (13A3) | QLQLQESGPGLVKPSETLSLTCTVSGGSISSRSYYWGWIRQPPGKGLEWIGSTYYSGET |
| | IgG1.1f (N60A) | YYAPSLKSRVTISVDTSKNQFSLKLSSVTAADTAVYYCATGGPYGDYAHWFDPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 85 | TIM3.13 (13A3) | QLQLQESGPGLVKPSETLSLTCTVSGGSISSRSYYWGWIRQPPGKGLEWIGSTYYSGET |
| | IgG1.1f (D101E) | YYNPSLKSRVTISVDTSKNQFSLKLSSVTAADTAVYYCATGGPYGDYAHWEEPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 86 | TIM3.14 (13A3) | QLQLQESGPGLVKPSETLSLTCTVSGGSISSRSYYWGWIRQPPGKGLEWIGSIYYSGFT |
| | IgG1.1f (P102V) | YYNPSLKSRVTISVDTSKNQFSLKLSSVTAADTAVYYCATGGPYGDYAHWFDVWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 87 | TIM3.15 (13A3) | QLQLQESGPGLVKPSETLSLTCTVSGGSISSRSYYWGWIRQPPGKGLEWIGSIYYSGFT |
| | IgG1.1f (P102Y) | YYNPSLKSRVTISVDTSKNQFSLKLSSVTAADTAVYYCATGGPYGDYAHWFDYWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 88 | TIM3.16 (13A3) | QLQLQESGPGLVKPSETLSLTCTVSGGSISSRSYYWGWIRQPPGKGLEWIGSIYYSGFT |
| | IgG1.1f (P102L) | YYNPSLKSRVTISVDTSKNQFSLKLSSVTAADTAVYYCATGGPYGDYAHWFDLWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 89 | TIM3.17 (13A3) | QLQLQESGPGLVKPSETLSLTCTVSGGSISSRSYYWGWIRQPPGKGLEWIGSIYYSGFT |
| | IgG1.1f | YYQPSLKSRVTISVDTSKNQFSLKLSSVTAADTAVYYCATGGPYGDYAHWFDYWGQGTL |
| | (N60Q/P102Y) | VTVSSASTKGPSVFPLAPSSKSTSGGTAALGCLVKDYFPEPVTVSWNSGALTSGVHTFP |
| | Heavy Chain (no | AVLQSSGLYSLSSVVTVPSSSLGTQTYICNVNHKPSNTKVDKRVEPKSCDKTHTCPPCP |
| | C-terminal K) | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 350 | TIM3.18 (13A3) | QLQLQESGPGLVKPSETLSLTCTVSGGSISSRSYYWGWIRQPPGKGLEWIGSIYYSGFT |
| | IgG1.1f | YYQPSLKSRVTISVDTSKNQFSLKLSSVTAADTAVYYCATGGPYGDYAHWFEPWGQGTL |
| | (N60Q/D101E) | VTVSSASTKGPSVFPLAPSSKSTSGGTAALGCLVKDYFPEPVTVSWNSGALTSGVHTFP |
| | Heavy Chain (no | AVLQSSGLYSLSSVVTVPSSSLGTQTYICNVNHKPSNTKVDKRVEPKSCDKTHTCPPCP |
| | C-terminal K) | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 90 | TIM3.8 (8B9) | QVQLQESGPGLVKPSETLSLTCTVSGGSISRHYWNWIRQPPGKGLEWIGYIHYSGSTNY |
| | IgG1.1f (S61P) | NPSLKSRVTISVDTSKNQFSLKLSSVTAADTAVYYCARDTGYYGMDIWGQGTTVTVSSA |
| | Heavy Chain (no | STKGPSVFPLAPSSKSTSGGTAALGCLVKDYFPEPVTVSWNSGALTSGVHTFPAVLQSS |
| | C-terminal K) | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPSSIEKTISKAKGQPREPQVYTLPPS |
| | | REEMTKNQVSLTCLVKGFYPSDIAVEWESNGQPENNYKTTPPVLDSDGSFFLYSKLTVD |
| | | KSRWQQGNVFSCSVMHEALHNHYTQKSLSLSPG* |
| 91 | TIM3.7 (9F6) | QVQLVESGGGLVKPGGSLRLSCAASGFTFSDYYMSWIRQAPGKGLEWVSFISGGGSTIY |
| | IgG1.1f (A108T) | YADSVKGRFTISRDNAKNSLFLQMNSLRVEDTAVYYCARDGYSSGWYYYGMDVWGQGTT |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 389 | TIM3.24/14H7 | QVHLVESGGGLVKPGGSLRLSCTAFSDYYMSWIRQAPGKGLEWLSYISNSGSITYYADS |
| | IgG1.1f Heavy | VKGRETISRDNAKNSVYLQMNSLRAEDTAVYYCARGRIGFEDYWGPGTLVTVSSASTKG |
| | Chain (no C- | PSVFPLAPSSKSTSGGTAALGCLVKDYFPEPVTVSWNSGALTSGVHTFPAVLQSSGLYS |
| | terminal K) | LSSVVTVPSSSLGTQTYICNVNHKPSNTKVDKRVEPKSCDKTHTCPPCPAPEAEGAPSV |
| | | FLFPPKPKDTLMISRTPEVTCVVVDVSHEDPEVKFNWYVDGVEVHNAKTKPREEQYNST |
| | | YRVVSVLTVLHQDWLNGKEYKCKVSNKALPSSIEKTISKAKGQPREPQVYTLPPSREEM |
| | | TKNQVSLTCLVKGFYPSDIAVEWESNGQPENNYKTTPPVLDSDGSFELYSKLTVDKSRW |
| | | QQGNVFSCSVMHEALHNHYTQKSLSLSPG* |
| 397 | 23B3 IgG1.1f | QVQLVGSGGGLVKPGGSLRLSCAASGFTESDYYMSWIRQAPGKGLEWVSFISGSGSITY |
| | Heavy Chain (no | YADSVKGRETISRDNAKNSLDLQMNSLRAEDTAVYYCARDGMVRGMNFYGMDVWGQGTT |
| | C-terminal K) | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 403 | TIM3.25 (23B3) | QVQLVESGGGLVKPGGSLRLSCAASGFTESDYYMSWIRQAPGKGLEWVSFISGSGSITY |
| | IgG1.1f (G6E, | YADSVKGRETISRDNAKNSLYLQMNSLRAEDTAVYYCARDGMVRGMNFYGMDVWGQGTT |
| | D79Y) Heavy | VTVSSASTKGPSVFPLAPSSKSTSGGTAALGCLVKDYFPEPVTVSWNSGALTSGVHTFP |
| | Chain (no C- | AVLQSSGLYSLSSVVTVPSSSLGTQTYICNVNHKPSNTKVDKRVEPKSCDKTHTCPPCP |
| | terminal K) | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPSSIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 92 | TIM3.10 (13A3) | QLQLQESGPGLVKPSETLSLTCTVSGGSISSRSYYWGWIRQPPGKGLEWIGSTYYSGET |
| | IgG1.3f (N60Q) | YYQPSLKSRVTISVDTSKNQFSLKLSSVTAADTAVYYCATGGPYGDYAHWFDPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 93 | TIM3.11 (13A3) | QLQLQESGPGLVKPSETLSLTCTVSGGSISSRSYYWGWIRQPPGKGLEWIGSTYYSGET |
| | IgG1.3f (N60S) | YYSPSLKSRVTISVDTSKNQFSLKLSSVTAADTAVYYCATGGPYGDYAHWFDPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 94 | TIM3.12 (13A3) | QLQLQESGPGLVKPSETLSLTCTVSGGSISSRSYYWGWIRQPPGKGLEWIGSTYYSGET |
| | IgG1.3f (N60A) | YYAPSLKSRVTISVDTSKNQFSLKLSSVTAADTAVYYCATGGPYGDYAHWFDPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 95 | TIM3.13 (13A3) | QLQLQESGPGLVKPSETLSLTCTVSGGSISSRSYYWGWIRQPPGKGLEWIGSTYYSGET |
| | IgG1.3f (D101E) | YYNPSLKSRVTISVDTSKNQFSLKLSSVTAADTAVYYCATGGPYGDYAHWEEPWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 96 | TIM3.14 (13A3) | QLQLQESGPGLVKPSETLSLTCTVSGGSISSRSYYWGWIRQPPGKGLEWIGSTYYSGET |
| | IgG1.3f (P102V) | YYNPSLKSRVTISVDTSKNQFSLKLSSVTAADTAVYYCATGGPYGDYAHWEDVWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 97 | TIM3.15 (13A3) | QLQLQESGPGLVKPSETLSLTCTVSGGSISSRSYYWGWIRQPPGKGLEWIGSTYYSGET |
| | IgG1.3f (P102Y) | YYNPSLKSRVTISVDTSKNQFSLKLSSVTAADTAVYYCATGGPYGDYAHWFDYWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 98 | TIM3.16 (13A3) | QLQLQESGPGLVKPSETLSLTCTVSGGSISSRSYYWGWIRQPPGKGLEWIGSTYYSGET |
| | IgG1.3f (P102L) | YYNPSLKSRVTISVDTSKNQFSLKLSSVTAADTAVYYCATGGPYGDYAHWFDLWGQGTL |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 99 | TIM3.17 (13A3) | QLQLQESGPGLVKPSETLSLTCTVSGGSISSRSYYWGWIRQPPGKGLEWIGSTYYSGET |
| | IgG1.3f | YYQPSLKSRVTISVDTSKNQFSLKLSSVTAADTAVYYCATGGPYGDYAHWFDYWGQGTL |
| | (N60Q/P102Y) | VTVSSASTKGPSVFPLAPSSKSTSGGTAALGCLVKDYFPEPVTVSWNSGALTSGVHTFP |
| | Heavy Chain | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 351 | TIM3.18 (13A3) | QLQLQESGPGLVKPSETLSLTCTVSGGSISSRSYYWGWIRQPPGKGLEWIGSTYYSGET |
| | IgG1.3f | YYQPSLKSRVTISVDTSKNQFSLKLSSVTAADTAVYYCATGGPYGDYAHWEEPWGQGTL |
| | (N60Q/D101E) | VTVSSASTKGPSVFPLAPSSKSTSGGTAALGCLVKDYFPEPVTVSWNSGALTSGVHTFP |
| | Heavy Chain | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 100 | TIM3.8 (8B9) | QVQLQESGPGLVKPSETLSLTCTVSGGSISRHYWNWIRQPPGKGLEWIGYIHYSGSTNY |
| | IgG1.3f (S61P) | NPSLKSRVTISVDTSKNQFSLKLSSVTAADTAVYYCARDTGYYGMDIWGQGTTVTVSSA |
| | Heavy Chain | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFELYSKLTVD |
| | | KSRWQQGNVFSCSVMHEALHNHYTQKSLSLSPGK* |
| 101 | TIM3.7 (9F6) | QVQLVESGGGLVKPGGSLRLSCAASGFTESDYYMSWIRQAPGKGLEWVSFISGGGSTIY |
| | IgG1.3f (A108T) | YADSVKGRFTISRDNAKNSLFLQMNSLRVEDTAVYYCARDGYSSGWYYYGMDVWGQGTT |
| | Heavy Chain | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 390 | TIM3.24/14H7 | QVHLVESGGGLVKPGGSLRLSCTAFSDYYMSWIRQAPGKGLEWLSYISNSGSITYYADS |
| | IgG1.3f Heavy | VKGRETISRDNAKNSVYLQMNSLRAEDTAVYYCARGRIGFEDYWGPGTLVTVSSASTKG |
| | Chain | PSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKRVEPKSCDKTHTCPPCPAPEAEGAPSV |
| | | FLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFELYSKLTVDKSRW |
| | | QQGNVFSCSVMHEALHNHYTQKSLSLSPGK* |
| 398 | 23B3 IgG1.3f | QVQLVGSGGGLVKPGGSLRLSCAASGFTESDYYMSWIRQAPGKGLEWVSFISGSGSITY |
| | Heavy Chain | YADSVKGRETISRDNAKNSLDLQMNSLRAEDTAVYYCARDGMVRGMNFYGMDVWGQGTT |
| | | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 404 | TIM3.25 (23B3) | QVQLVESGGGLVKPGGSLRLSCAASGFTESDYYMSWIRQAPGKGLEWVSFISGSGSITY |
| | IgG1.3f (G6E, | YADSVKGRETISRDNAKNSLYLQMNSLRAEDTAVYYCARDGMVRGMNFYGMDVWGQGTT |
| | D79Y) Heavy | VTVSSASTKGPSVFPLAPSSKSTSGGTAALGCLVKDYFPEPVTVSWNSGALTSGVHTFP |
| | Chain | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPGK* |
| 102 | TIM3.10 (13A3) | QLQLQESGPGLVKPSETLSLTCTVSGGSISSRSYYWGWIRQPPGKGLEWIGSTYYSGET |
| | IgG1.3f (N60Q) | YYQPSLKSRVTISVDTSKNQFSLKLSSVTAADTAVYYCATGGPYGDYAHWFDPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 103 | TIM3.11 (13A3) | QLQLQESGPGLVKPSETLSLTCTVSGGSISSRSYYWGWIRQPPGKGLEWIGSTYYSGET |
| | IgG1.3f (N60S) | YYSPSLKSRVTISVDTSKNQFSLKLSSVTAADTAVYYCATGGPYGDYAHWFDPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 104 | TIM3.12 (13A3) | QLQLQESGPGLVKPSETLSLTCTVSGGSISSRSYYWGWIRQPPGKGLEWIGSTYYSGET |
| | IgG1.3f (N60A) | YYAPSLKSRVTISVDTSKNQFSLKLSSVTAADTAVYYCATGGPYGDYAHWFDPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 105 | TIM3.13 (13A3) | QLQLQESGPGLVKPSETLSLTCTVSGGSISSRSYYWGWIRQPPGKGLEWIGSTYYSGET |
| | IgG1.3f (D101E) | YYNPSLKSRVTISVDTSKNQFSLKLSSVTAADTAVYYCATGGPYGDYAHWEEPWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 106 | TIM3.14 (13A3) | QLQLQESGPGLVKPSETLSLTCTVSGGSISSRSYYWGWIRQPPGKGLEWIGSIYYSGFT |
| | IgG1.3f (P102V) | YYNPSLKSRVTISVDTSKNQFSLKLSSVTAADTAVYYCATGGPYGDYAHWFDVWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 107 | TIM3.15 (13A3) | QLQLQESGPGLVKPSETLSLTCTVSGGSISSRSYYWGWIRQPPGKGLEWIGSIYYSGFT |
| | IgG1.3f (P102Y) | YYNPSLKSRVTISVDTSKNQFSLKLSSVTAADTAVYYCATGGPYGDYAHWFDYWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 108 | TIM3.16 (13A3) | QLQLQESGPGLVKPSETLSLTCTVSGGSISSRSYYWGWIRQPPGKGLEWIGSIYYSGFT |
| | IgG1.3f (P102L) | YYNPSLKSRVTISVDTSKNQFSLKLSSVTAADTAVYYCATGGPYGDYAHWFDLWGQGTL |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 109 | TIM3.17 (13A3) | QLQLQESGPGLVKPSETLSLTCTVSGGSISSRSYYWGWIRQPPGKGLEWIGSIYYSGFT |
| | IgG1.3f | YYQPSLKSRVTISVDTSKNQFSLKLSSVTAADTAVYYCATGGPYGDYAHWFDYWGQGTL |
| | (N60Q/P102Y) | VTVSSASTKGPSVFPLAPSSKSTSGGTAALGCLVKDYFPEPVTVSWNSGALTSGVHTFP |
| | Heavy Chain (no | AVLQSSGLYSLSSVVTVPSSSLGTQTYICNVNHKPSNTKVDKRVEPKSCDKTHTCPPCP |
| | C-terminal K) | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 352 | TIM3.18 (13A3) | QLQLQESGPGLVKPSETLSLTCTVSGGSISSRSYYWGWIRQPPGKGLEWIGSIYYSGFT |
| | IgG1.3f | YYQPSLKSRVTISVDTSKNQFSLKLSSVTAADTAVYYCATGGPYGDYAHWFEPWGQGTL |
| | (N60Q/D101E) | VTVSSASTKGPSVFPLAPSSKSTSGGTAALGCLVKDYFPEPVTVSWNSGALTSGVHTFP |
| | Heavy Chain (no | AVLQSSGLYSLSSVVTVPSSSLGTQTYICNVNHKPSNTKVDKRVEPKSCDKTHTCPPCP |
| | C-terminal K) | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 110 | TIM3.8 (8B9) | QVQLQESGPGLVKPSETLSLTCTVSGGSISRHYWNWIRQPPGKGLEWIGYIHYSGSTNY |
| | IgG1.3f (S61P) | NPSLKSRVTISVDTSKNQFSLKLSSVTAADTAVYYCARDTGYYGMDIWGQGTTVTVSSA |
| | Heavy Chain (no | STKGPSVFPLAPSSKSTSGGTAALGCLVKDYFPEPVTVSWNSGALTSGVHTFPAVLQSS |
| | C-terminal K) | GLYSLSSVVTVPSSSLGTQTYICNVNHKPSNTKVDKRVEPKSCDKTHTCPPCPAPEAEG |
| | | APSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSDGSFFLYSKLTVD |
| | | KSRWQQGNVFSCSVMHEALHNHYTQKSLSLSPG* |
| 111 | TIM3.7 (9F6) | QVQLVESGGGLVKPGGSLRLSCAASGFTFSDYYMSWIRQAPGKGLEWVSFISGGGSTIY |
| | IgG1.3f (A108T) | YADSVKGRFTISRDNAKNSLFLQMNSLRVEDTAVYYCARDGYSSGWYYYGMDVWGQGTT |
| | Heavy Chain (no | VTVSSASTKGPSVFPLAPSSKSTSGGTAALGCLVKDYFPEPVTVSWNSGALTSGVHTFP |
| | C-terminal K) | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG* |
| 391 | TIM3.24/14H7 | QVHLVESGGGLVKPGGSLRLSCTAFSDYYMSWIRQAPGKGLEWLSYISNSGSITYYADS |
| | IgG1.3f Heavy | VKGRETISRDNAKNSVYLQMNSLRAEDTAVYYCARGRIGFEDYWGPGTLVTVSSASTKG |
| | Chain (no C- | PSVFPLAPSSKSTSGGTAALGCLVKDYFPEPVTVSWNSGALTSGVHTFPAVLQSSGLYS |
| | terminal K) | LSSVVTVPSSSLGTQTYICNVNHKPSNTKVDKRVEPKSCDKTHTCPPCPAPEAEGAPSV |
| | | FLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFELYSKLTVDKSRW |
| | | QQGNVFSCSVMHEALHNHYTQKSLSLSPG* |
| 399 | 23B3 IgG1.1f | QVQLVGSGGGLVKPGGSLRLSCAASGFTESDYYMSWIRQAPGKGLEWVSFISGSGSITY |
| | Heavy Chain (no | YADSVKGRETISRDNAKNSLDLQMNSLRAEDTAVYYCARDGMVRGMNFYGMDVWGQGTT |
| | C-terminal K) | VTVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEPKSCDKTHTCPPCP |
| | | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 405 | TIM3.25 (23B3) | QVQLVESGGGLVKPGGSLRLSCAASGFTESDYYMSWIRQAPGKGLEWVSFISGSGSITY |
| | IgG1.1f (G6E, | YADSVKGRETISRDNAKNSLYLQMNSLRAEDTAVYYCARDGMVRGMNFYGMDVWGQGTT |
| | D79Y) Heavy | VTVSSASTKGPSVFPLAPSSKSTSGGTAALGCLVKDYFPEPVTVSWNSGALTSGVHTFP |
| | Chain (no C- | AVLQSSGLYSLSSVVTVPSSSLGTQTYICNVNHKPSNTKVDKRVEPKSCDKTHTCPPCP |
| | terminal K) | APEAEGAPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTTPPVLDSDGSFELY |
| | | SKLTVDKSRWQQGNVFSCSVMHEALHNHYTQKSLSLSPG* |
| 29 | TIM3.5 (13A3), | EIVLTQSPGTLSLSPGERATLSCRASQSVSSSYLAWYQQKPGQAPRLLIYGASSRATGI |
| | TIM3.2 (17C3), | PDRFSGSGSGTDFTLTISRLEPEDFAVYYCQQYGSSPITEGQGTRLEIKRTVAAPSVFI |
| | TIM3.4 (3G4), | FPPSDEQLKSGTASVVCLLNNFYPREAKVQWKVDNALQSGNSQESVTEQDSKDSTYSLS |
| | TIM3.25/23B3 | STLTLSKADYEKHKVYACEVTHQGLSSPVTKSFNRGEC* |
| | (VK1) IgG1 | |
| | Light Chain | |
| 30 | 8B9, TIM3.6 | EIVLTQSPGTLSLSPGERATLSCRASQSVSSSYLAWYQQKPGQAPRLLIYGASSRATGI |
| | (8C4), TIM3.9 | PDRFSGSGSGTDFTLTISRLEPEDFAVYYCQQYGSSPLTEGGGTKVEIKRTVAAPSVFI |
| | (17C8) IgG1 | FPPSDEQLKSGTASVVCLLNNFYPREAKVQWKVDNALQSGNSQESVTEQDSKDSTYSLS |
| | Light Chain | STLTLSKADYEKHKVYACEVTHQGLSSPVTKSFNRGEC* |
| 32 | 9F6 VK1 IgG1 | AIQLTQSPSSLSASVGDRVTITCRASQGISSALAWYQQKPGKAPKLLIYDASSLESGVP |
| | Light Chain | SRFSGSGSGTDFTLTISSLQPEDFATYYCQQFNSYPRTFGQGTKVEIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC* |
| 33 | 9F6 VK2 IgG1 | EIVLTQSPGTLSLSPGERATLSCRASQSVSSSYLAWYQQKPGQAPRLLIYGASSRATGI |
| | Light Chain | PDRFSGSGSGTDFTLTISRLEPEDFAVYYCQQYGSSLTEGGGTKVEIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC* |
| 31 | 9F6 VK3 IgG1 | EIVLTQSPGTLSLSPGERATLSCRASQSVSSSYLAWYQQKPGQAPRLLIYGASSRATGI |
| | Light Chain | PDRFSGSGSGTDFTLTISRLEPEDFAVYYCQQYGSSPLTEGGGTKVEIKRTVAAPSVFI |
| | | FPPSDEQLKSGTASVVCLLNNFYPREAKVQWKVDNALQSGNSQESVTEQDSKDSTYSLS |
| | | STLTLSKADYEKHKVYACEVTHQGLSSPVTKSFNRGEC* |
| 408 | TIM3.24/14H7 | EIVLTQSPGTLSLSPGERATLSCRASQSVSSSYLAWYQQKPGQAPRLLIYGASSRATGI |
| | IgG1 Light | PDRFSGSGSGTDFTLTISRLEPEDFAVYYCQQYGSSPLTEGPGTKVDIKRTVAAPSVFI |
| | Chain | FPPSDEQLKSGTASVVCLLNNFYPREAKVQWKVDNALQSGNSQESVTEQDSKDSTYSLS |
| | | STLTLSKADYEKHKVYACEVTHQGLSSPVTKSFNRGEC* |
| 409 | 23B3 IgG1 Light | EIVLTQSPGTLSLSPGERATLSCRASQSVSSSYLAWYQQKPGQAPRLLIYGASSRATGI |
| | Chain (VK2) | PDRFSGSGSGTDFTLTISRLEPEDFAVYYCQQYGSSPEGGGTKVEIKRTVAAPSVFIFP |
| | | PSDEQLKSGTASVVCLLNNEYPREAKVQWKVDNALQSGNSQESVTEQDSKDSTYSLSST |
| | | LTLSKADYEKHKVYACEVTHQGLSSPVTKSFNRGEC |
| 134 | TIM3.5 (13A3) | CAGCTGCAGCTGCAGGAGTCGGGCCCAGGACTGGTGAAGCCTTCGGAGACCCTGTCCCT |
| | IgG1.1f Heavy | CACCTGCACTGTCTCTGGTGGCTCCATCAGCAGTAGAAGTTACTACTGGGGCTGGATCC |
| | Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 135 | 8B9 IgG1.1f | CAGGTGCAGCTGCAGGAGTCGGGCCCAGGACTGGTGAAGCCTTCGGAGACCCTGTCCCT |
| | Heavy Chain | CACCTGCACTGTCTCTGGTGGCTCCATCAGTCGTCACTACTGGAACTGGATCCGGCAGC |
| | | CCCCAGGGAAGGGACTGGAGTGGATTGGGTATATCCATTACAGTGGAAGCACCAACTAC |
| | | AATTCCTCCCTCAAGAGTCGAGTCACCATATCAGTAGACACGTCCAAGAACCAGTTCTC |
| | | CCTGAAGCTGAGCTCTGTGACCGCTGCGGACACGGCCGTGTATTACTGTGCGAGAGATA |
| | | CTGGGTACTACGGTATGGACATCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAAGCAGCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTAAATGA |
| 136 | TIM3.6 (8C4) | CAGGTGCAGCTGCAGGAGTCGGGCCCAGGACTGGTGAAGCCTTCGGAGACCCTGTCCCT |
| | IgG1.1f Heavy | CACCTGCACTGTCTCTGGTGGCTCCATCAGTCGTTACTACTGGAGCTGGATCCGGCAGC |
| | Chain | CCCCAGGGAAGGGACTGGAGTGGATTGGGTATATCCATTACACTGGGAGCACCAACTAC |
| | | AACCCCTCCCTCAAGAGTCGAGTCACCATATCAGTAGACACGTCCAAGAACCAGTTCTC |
| | | CCTGAAGCTGAGCTCTGTGACCGCAGCGGACACGGCCGTGTATTACTGTGCGACAGATA |
| | | CGGGCTACTACGGTATGGACGTCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAAGCAGCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTAAATGA |
| 137 | TIM3.2 (17C3) | CAGGTGCAGTTGGTGCAGTCTGGGGCTGAGGTGAAGAAGCCTGGGGCCTCAGTGAAGGT |
| | IgG1.1f Heavy | CTCCTGCAAGGCATCTGGATACACTTTCACCAGCTACTATATGCACTGGGTGCGACAGG |
| | Chain | CCCCTGGACAAGGGCTTGAGTGGATGGGAATAATCAACCCTAGGGGTGATAGCATAATC |
| | | TACGCACAGAAGTTCCAGGGCAGAGTCACCATGACCAGGGACACGTCCACGAGCACAGT |
| | | CTACATGGAGCTGAGCAGCCTGAGATCTGAGGACACGGCCGTGTATTACTGTGCGAGAG |
| | | ATTTCTATGGTTCGGGAAACTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 138 | 9F6 IgG1.1f | CAGGTGCAGCTGGTGGAGTCTGGGGGAGGCTTGGTCAAGCCTGGAGGGTCCCTGAGACT |
| | Heavy Chain | CTCCTGTGCAGCCTCTGGATTCACCTTCAGTGACTACTACATGAGCTGGATCCGCCAGG |
| | | CTCCAGGGAAGGGGCTGGAGTGGGTTTCATTCATTAGTGGTGGTGGTAGTACCATATAC |
| | | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCGCG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 139 | TIM3.4 (3G4) | CAGGTGCAGCTGGTGGAGTCTGGGGGAGGCTTGGTCAAGCCTGGAGGGTCCCTGAGACT |
| | IgG1.1f Heavy | CTCCTGTGCAGCCTCTGGATTCACCTTCAGTGACTACTACATGAGCTGGATCCGCCAGG |
| | Chain | CTCCAGGGAAGGGGCTGGAGTGGGTTTCATTCATTAGTACTAGTGGTAGTATCATATAC |
| | | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | AAGGGTATAGCAGCAGCTGGTCCTACTACTACGGTATGGACGTCTGGGGCCAAGGGACC |
| | | ACGGTCACCGTCTCCTCAGCTAGCACCAAGGGCCCATCGGTCTTCCCCCTGGCACCCTC |
| | | CTCCAAGAGCACCTCTGGGGGCACAGCGGCCCTGGGCTGCCTGGTCAAGGACTACTTCC |
| | | CCGAACCGGTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCGTGCCCTC |
| | | CAGCAGCTTGGGCACCCAGACCTACATCTGCAACGTGAATCACAAGCCCAGCAACACCA |
| | | AGGTGGACAAGAGAGTTGAGCCCAAATCTTGTGACAAAACTCACACATGCCCACCGTGC |
| | | CCAGCACCTGAAGCCGAAGGGGCCCCGTCAGTCTTCCTCTTCCCCCCAAAACCCAAGGA |
| | | CACCCTCATGATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCATAATGCCAAG |
| | | ACAAAGCCGCGGGAGGAGCAGTACAACAGCACGTACCGTGTGGTCAGCGTCCTCACCGT |
| | | CCTGCACCAGGACTGGCTGAATGGCAAGGAGTACAAGTGCAAGGTCTCCAACAAAGCCC |
| | | TCCCAAGCAGCATCGAGAAAACCATCTCCAAAGCCAAAGGGCAGCCCCGAGAACCACAG |
| | | GTGTACACCCTGCCCCCATCCCGGGAGGAGATGACCAAGAACCAGGTCAGCCTGACCTG |
| | | CCTGGTCAAAGGCTTCTATCCCAGCGACATCGCCGTGGAGTGGGAGAGCAATGGGCAGC |
| | | CGGAGAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCCTGTCCCCGG |
| | | GTAAATGA |
| 140 | TIM3.9 (17C8) | CAGGTGCAGCTGGTGGAGTCTGGGGGAGGCTTGGTCAAGCCTGGAGGGTCCCTGAGACT |
| | IgG1.1f Heavy | CTCCTGTGCAGCCTCTGGATTCACCTTCAGTGACTACTACATGAGCTGGATCCGCCAGG |
| | Chain | CTCCAGGGAAGGGGCTGGAGTGGGTTTCATTCATTAGTAGTAGTGGTAGTATCATATAC |
| | | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | ATGGGTATAGCAGTGGCTGGGAGTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 141 | TIM3.5 (13A3) | CAGCTGCAGCTGCAGGAGTCGGGCCCAGGACTGGTGAAGCCTTCGGAGACCCTGTCCCT |
| | IgG1.1f Heavy | CACCTGCACTGTCTCTGGTGGCTCCATCAGCAGTAGAAGTTACTACTGGGGCTGGATCC |
| | Chain (without | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 142 | 8B9 IgG1.1f | CAGGTGCAGCTGCAGGAGTCGGGCCCAGGACTGGTGAAGCCTTCGGAGACCCTGTCCCT |
| | Heavy Chain | CACCTGCACTGTCTCTGGTGGCTCCATCAGTCGTCACTACTGGAACTGGATCCGGCAGC |
| | (without C- | CCCCAGGGAAGGGACTGGAGTGGATTGGGTATATCCATTACAGTGGAAGCACCAACTAC |
| | terminal K) | AATTCCTCCCTCAAGAGTCGAGTCACCATATCAGTAGACACGTCCAAGAACCAGTTCTC |
| | | CCTGAAGCTGAGCTCTGTGACCGCTGCGGACACGGCCGTGTATTACTGTGCGAGAGATA |
| | | CTGGGTACTACGGTATGGACATCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAAGCAGCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTTGA |
| 143 | TIM3.6 (8C4) | CAGGTGCAGCTGCAGGAGTCGGGCCCAGGACTGGTGAAGCCTTCGGAGACCCTGTCCCT |
| | IgG1.1f Heavy | CACCTGCACTGTCTCTGGTGGCTCCATCAGTCGTTACTACTGGAGCTGGATCCGGCAGC |
| | Chain (without | CCCCAGGGAAGGGACTGGAGTGGATTGGGTATATCCATTACACTGGGAGCACCAACTAC |
| | C-terminal K) | AACCCCTCCCTCAAGAGTCGAGTCACCATATCAGTAGACACGTCCAAGAACCAGTTCTC |
| | | CCTGAAGCTGAGCTCTGTGACCGCAGCGGACACGGCCGTGTATTACTGTGCGACAGATA |
| | | CGGGCTACTACGGTATGGACGTCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAAGCAGCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTTGA |
| 144 | TIM3.2 (17C3) | CAGGTGCAGTTGGTGCAGTCTGGGGCTGAGGTGAAGAAGCCTGGGGCCTCAGTGAAGGT |
| | IgG1.1f Heavy | CTCCTGCAAGGCATCTGGATACACTTTCACCAGCTACTATATGCACTGGGTGCGACAGG |
| | Chain (without | CCCCTGGACAAGGGCTTGAGTGGATGGGAATAATCAACCCTAGGGGTGATAGCATAATC |
| | C-terminal K) | TACGCACAGAAGTTCCAGGGCAGAGTCACCATGACCAGGGACACGTCCACGAGCACAGT |
| | | CTACATGGAGCTGAGCAGCCTGAGATCTGAGGACACGGCCGTGTATTACTGTGCGAGAG |
| | | ATTTCTATGGTTCGGGAAACTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 145 | 9F6 IgG1.1f | CAGGTGCAGCTGGTGGAGTCTGGGGGAGGCTTGGTCAAGCCTGGAGGGTCCCTGAGACT |
| | Heavy Chain | CTCCTGTGCAGCCTCTGGATTCACCTTCAGTGACTACTACATGAGCTGGATCCGCCAGG |
| | (without C- | CTCCAGGGAAGGGGCTGGAGTGGGTTTCATTCATTAGTGGTGGTGGTAGTACCATATAC |
| | terminal K) | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCGCG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 146 | TIM3.4 (3G4) | CAGGTGCAGCTGGTGGAGTCTGGGGGAGGCTTGGTCAAGCCTGGAGGGTCCCTGAGACT |
| | IgG1.1f Heavy | CTCCTGTGCAGCCTCTGGATTCACCTTCAGTGACTACTACATGAGCTGGATCCGCCAGG |
| | Chain (without | CTCCAGGGAAGGGGCTGGAGTGGGTTTCATTCATTAGTACTAGTGGTAGTATCATATAC |
| | C-terminal K) | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | AAGGGTATAGCAGCAGCTGGTCCTACTACTACGGTATGGACGTCTGGGGCCAAGGGACC |
| | | ACGGTCACCGTCTCCTCAGCTAGCACCAAGGGCCCATCGGTCTTCCCCCTGGCACCCTC |
| | | CTCCAAGAGCACCTCTGGGGGCACAGCGGCCCTGGGCTGCCTGGTCAAGGACTACTTCC |
| | | CCGAACCGGTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCGTGCCCTC |
| | | CAGCAGCTTGGGCACCCAGACCTACATCTGCAACGTGAATCACAAGCCCAGCAACACCA |
| | | AGGTGGACAAGAGAGTTGAGCCCAAATCTTGTGACAAAACTCACACATGCCCACCGTGC |
| | | CCAGCACCTGAAGCCGAAGGGGCCCCGTCAGTCTTCCTCTTCCCCCCAAAACCCAAGGA |
| | | CACCCTCATGATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCATAATGCCAAG |
| | | ACAAAGCCGCGGGAGGAGCAGTACAACAGCACGTACCGTGTGGTCAGCGTCCTCACCGT |
| | | CCTGCACCAGGACTGGCTGAATGGCAAGGAGTACAAGTGCAAGGTCTCCAACAAAGCCC |
| | | TCCCAAGCAGCATCGAGAAAACCATCTCCAAAGCCAAAGGGCAGCCCCGAGAACCACAG |
| | | GTGTACACCCTGCCCCCATCCCGGGAGGAGATGACCAAGAACCAGGTCAGCCTGACCTG |
| | | CCTGGTCAAAGGCTTCTATCCCAGCGACATCGCCGTGGAGTGGGAGAGCAATGGGCAGC |
| | | CGGAGAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCCTGTCCCCGG |
| | | GTTGA |
| 147 | TIM3.9 (17C8) | CAGGTGCAGCTGGTGGAGTCTGGGGGAGGCTTGGTCAAGCCTGGAGGGTCCCTGAGACT |
| | IgG1.1f Heavy | CTCCTGTGCAGCCTCTGGATTCACCTTCAGTGACTACTACATGAGCTGGATCCGCCAGG |
| | Chain (without | CTCCAGGGAAGGGGCTGGAGTGGGTTTCATTCATTAGTAGTAGTGGTAGTATCATATAC |
| | C-terminal K) | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | ATGGGTATAGCAGTGGCTGGGAGTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 148 | TIM3.5 (13A3) | CAGCTGCAGCTGCAGGAGTCGGGCCCAGGACTGGTGAAGCCTTCGGAGACCCTGTCCCT |
| | IgG1.3f Heavy | CACCTGCACTGTCTCTGGTGGCTCCATCAGCAGTAGAAGTTACTACTGGGGCTGGATCC |
| | Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 149 | 8B9 IgG1.3f | AGGTGCAGCTGCAGGAGTCGGGCCCAGGACTGGTGAAGCCTTCGGAGACCCTGTCCCTC |
| | Heavy Chain | ACCTGCACTGTCTCTGGTGGCTCCATCAGTCGTCACTACTGGAACTGGATCCGGCAGCC |
| | | CCCAGGGAAGGGACTGGAGTGGATTGGGTATATCCATTACAGTGGAAGCACCAACTACA |
| | | ATTCCTCCCTCAAGAGTCGAGTCACCATATCAGTAGACACGTCCAAGAACCAGTTCTCC |
| | | CTGAAGCTGAGCTCTGTGACCGCTGCGGACACGGCCGTGTATTACTGTGCGAGAGATAC |
| | | TGGGTACTACGGTATGGACATCTGGGGCCAAGGGACCACGGTCACCGTCTCCTCAGCTA |
| | | GCACCAAGGGCCCATCGGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGTCGTG |
| | | GAACTCAGGCGCCCTGACCAGCGGCGTGCACACCTTCCCGGCTGTCCTACAGTCCTCAG |
| | | GACTCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTTGAGCC |
| | | CAAATCTTGTGACAAAACTCACACATGCCCACCGTGCCCAGCACCTGAAGCCGAAGGGG |
| | | CCCCGTCAGTCTTCCTCTTCCCCCCAAAACCCAAGGACACCCTCATGATCTCCCGGACC |
| | | CCTGAGGTCACATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGGGAGGAGCAGT |
| | | ACAACAGCACGTACCGTGTGGTCAGCGTCCTCACCGTCCTGCACCAGGACTGGCTGAAT |
| | | GGCAAGGAGTACAAGTGCAAGGTCTCCAACAAAGCCCTCCCAGCCCCCATCGAGAAAAC |
| | | CATCTCCAAAGCCAAAGGGCAGCCCCGAGAACCACAGGTGTACACCCTGCCCCCATCCC |
| | | GGGAGGAGATGACCAAGAACCAGGTCAGCCTGACCTGCCTGGTCAAAGGCTTCTATCCC |
| | | AGCGACATCGCCGTGGAGTGGGAGAGCAATGGGCAGCCGGAGAACAACTACAAGACCAC |
| | | GCCTCCCGTGCTGGACTCCGACGGCTCCTTCTTCCTCTATAGCAAGCTCACCGTGGACA |
| | | AGAGCAGGTGGCAGCAGGGGAACGTCTTCTCATGCTCCGTGATGCATGAGGCTCTGCAC |
| | | AACCACTACACGCAGAAGAGCCTCTCCCTGTCCCCGGGTAAATGA |
| 150 | TIM3.6 (8C4) | CAGGTGCAGCTGCAGGAGTCGGGCCCAGGACTGGTGAAGCCTTCGGAGACCCTGTCCCT |
| | IgG1.3f Heavy | CACCTGCACTGTCTCTGGTGGCTCCATCAGTCGTTACTACTGGAGCTGGATCCGGCAGC |
| | Chain | CCCCAGGGAAGGGACTGGAGTGGATTGGGTATATCCATTACACTGGGAGCACCAACTAC |
| | | AACCCCTCCCTCAAGAGTCGAGTCACCATATCAGTAGACACGTCCAAGAACCAGTTCTC |
| | | CCTGAAGCTGAGCTCTGTGACCGCAGCGGACACGGCCGTGTATTACTGTGCGACAGATA |
| | | CGGGCTACTACGGTATGGACGTCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAGCCCCCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTAAATGA |
| 151 | TIM3.2 (17C3) | CAGGTGCAGTTGGTGCAGTCTGGGGCTGAGGTGAAGAAGCCTGGGGCCTCAGTGAAGGT |
| | IgG1.3f Heavy | CTCCTGCAAGGCATCTGGATACACTTTCACCAGCTACTATATGCACTGGGTGCGACAGG |
| | Chain | CCCCTGGACAAGGGCTTGAGTGGATGGGAATAATCAACCCTAGGGGTGATAGCATAATC |
| | | TACGCACAGAAGTTCCAGGGCAGAGTCACCATGACCAGGGACACGTCCACGAGCACAGT |
| | | CTACATGGAGCTGAGCAGCCTGAGATCTGAGGACACGGCCGTGTATTACTGTGCGAGAG |
| | | ATTTCTATGGTTCGGGAAACTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 152 | 9F6 IgG1.3f | CAGGTGCAGCTGGTGGAGTCTGGGGGAGGCTTGGTCAAGCCTGGAGGGTCCCTGAGACT |
| | Heavy Chain | CTCCTGTGCAGCCTCTGGATTCACCTTCAGTGACTACTACATGAGCTGGATCCGCCAGG |
| | | CTCCAGGGAAGGGGCTGGAGTGGGTTTCATTCATTAGTGGTGGTGGTAGTACCATATAC |
| | | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCGCG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 153 | TIM3.4 (3G4) | CAGGTGCAGCTGGTGGAGTCTGGGGGAGGCTTGGTCAAGCCTGGAGGGTCCCTGAGACT |
| | IgG1.3f Heavy | CTCCTGTGCAGCCTCTGGATTCACCTTCAGTGACTACTACATGAGCTGGATCCGCCAGG |
| | Chain | CTCCAGGGAAGGGGCTGGAGTGGGTTTCATTCATTAGTACTAGTGGTAGTATCATATAC |
| | | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | AAGGGTATAGCAGCAGCTGGTCCTACTACTACGGTATGGACGTCTGGGGCCAAGGGACC |
| | | ACGGTCACCGTCTCCTCAGCTAGCACCAAGGGCCCATCGGTCTTCCCCCTGGCACCCTC |
| | | CTCCAAGAGCACCTCTGGGGGCACAGCGGCCCTGGGCTGCCTGGTCAAGGACTACTTCC |
| | | CCGAACCGGTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCGTGCCCTC |
| | | CAGCAGCTTGGGCACCCAGACCTACATCTGCAACGTGAATCACAAGCCCAGCAACACCA |
| | | AGGTGGACAAGAGAGTTGAGCCCAAATCTTGTGACAAAACTCACACATGCCCACCGTGC |
| | | CCAGCACCTGAAGCCGAAGGGGCCCCGTCAGTCTTCCTCTTCCCCCCAAAACCCAAGGA |
| | | CACCCTCATGATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCATAATGCCAAG |
| | | ACAAAGCCGCGGGAGGAGCAGTACAACAGCACGTACCGTGTGGTCAGCGTCCTCACCGT |
| | | CCTGCACCAGGACTGGCTGAATGGCAAGGAGTACAAGTGCAAGGTCTCCAACAAAGCCC |
| | | TCCCAGCCCCCATCGAGAAAACCATCTCCAAAGCCAAAGGGCAGCCCCGAGAACCACAG |
| | | GTGTACACCCTGCCCCCATCCCGGGAGGAGATGACCAAGAACCAGGTCAGCCTGACCTG |
| | | CCTGGTCAAAGGCTTCTATCCCAGCGACATCGCCGTGGAGTGGGAGAGCAATGGGCAGC |
| | | CGGAGAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCCTGTCCCCGG |
| | | GTAAATGA |
| 154 | TIM3.9 (17C8) | CAGGTGCAGCTGGTGGAGTCTGGGGGAGGCTTGGTCAAGCCTGGAGGGTCCCTGAGACT |
| | IgG1.3f Heavy | CTCCTGTGCAGCCTCTGGATTCACCTTCAGTGACTACTACATGAGCTGGATCCGCCAGG |
| | Chain | CTCCAGGGAAGGGGCTGGAGTGGGTTTCATTCATTAGTAGTAGTGGTAGTATCATATAC |
| | | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | ATGGGTATAGCAGTGGCTGGGAGTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 155 | TIM3.5 (13A3) | CAGCTGCAGCTGCAGGAGTCGGGCCCAGGACTGGTGAAGCCTTCGGAGACCCTGTCCCT |
| | IgG1.3f Heavy | CACCTGCACTGTCTCTGGTGGCTCCATCAGCAGTAGAAGTTACTACTGGGGCTGGATCC |
| | Chain (no C- | GCCAGCCCCCAGGGAAGGGGCTGGAGTGGATTGGGAGTATCTATTATAGTGGGTTCACC |
| | terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 156 | 8B9 IgG1.3f | AGGTGCAGCTGCAGGAGTCGGGCCCAGGACTGGTGAAGCCTTCGGAGACCCTGTCCCTC |
| | Heavy Chain (no | ACCTGCACTGTCTCTGGTGGCTCCATCAGTCGTCACTACTGGAACTGGATCCGGCAGCC |
| | C-terminal K) | CCCAGGGAAGGGACTGGAGTGGATTGGGTATATCCATTACAGTGGAAGCACCAACTACA |
| | | ATTCCTCCCTCAAGAGTCGAGTCACCATATCAGTAGACACGTCCAAGAACCAGTTCTCC |
| | | CTGAAGCTGAGCTCTGTGACCGCTGCGGACACGGCCGTGTATTACTGTGCGAGAGATAC |
| | | TGGGTACTACGGTATGGACATCTGGGGCCAAGGGACCACGGTCACCGTCTCCTCAGCTA |
| | | GCACCAAGGGCCCATCGGTCTTCCCCCTGGCACCCTCCTCCAAGAGCACCTCTGGGGGC |
| | | ACAGCGGCCCTGGGCTGCCTGGTCAAGGACTACTTCCCCGAACCGGTGACGGTGTCGTG |
| | | GAACTCAGGCGCCCTGACCAGCGGCGTGCACACCTTCCCGGCTGTCCTACAGTCCTCAG |
| | | GACTCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTTGAGCC |
| | | CAAATCTTGTGACAAAACTCACACATGCCCACCGTGCCCAGCACCTGAAGCCGAAGGGG |
| | | CCCCGTCAGTCTTCCTCTTCCCCCCAAAACCCAAGGACACCCTCATGATCTCCCGGACC |
| | | CCTGAGGTCACATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGGGAGGAGCAGT |
| | | ACAACAGCACGTACCGTGTGGTCAGCGTCCTCACCGTCCTGCACCAGGACTGGCTGAAT |
| | | GGCAAGGAGTACAAGTGCAAGGTCTCCAACAAAGCCCTCCCAGCCCCCATCGAGAAAAC |
| | | CATCTCCAAAGCCAAAGGGCAGCCCCGAGAACCACAGGTGTACACCCTGCCCCCATCCC |
| | | GGGAGGAGATGACCAAGAACCAGGTCAGCCTGACCTGCCTGGTCAAAGGCTTCTATCCC |
| | | AGCGACATCGCCGTGGAGTGGGAGAGCAATGGGCAGCCGGAGAACAACTACAAGACCAC |
| | | GCCTCCCGTGCTGGACTCCGACGGCTCCTTCTTCCTCTATAGCAAGCTCACCGTGGACA |
| | | AGAGCAGGTGGCAGCAGGGGAACGTCTTCTCATGCTCCGTGATGCATGAGGCTCTGCAC |
| | | AACCACTACACGCAGAAGAGCCTCTCCCTGTCCCCGGGTTGA |
| 157 | TIM3.6 (8C4) | CAGGTGCAGCTGCAGGAGTCGGGCCCAGGACTGGTGAAGCCTTCGGAGACCCTGTCCCT |
| | IgG1.3f Heavy | CACCTGCACTGTCTCTGGTGGCTCCATCAGTCGTTACTACTGGAGCTGGATCCGGCAGC |
| | Chain (no C- | CCCCAGGGAAGGGACTGGAGTGGATTGGGTATATCCATTACACTGGGAGCACCAACTAC |
| | terminal K) | AACCCCTCCCTCAAGAGTCGAGTCACCATATCAGTAGACACGTCCAAGAACCAGTTCTC |
| | | CCTGAAGCTGAGCTCTGTGACCGCAGCGGACACGGCCGTGTATTACTGTGCGACAGATA |
| | | CGGGCTACTACGGTATGGACGTCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAGCCCCCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTTGA |
| 158 | TIM3.2 (17C3) | CAGGTGCAGTTGGTGCAGTCTGGGGCTGAGGTGAAGAAGCCTGGGGCCTCAGTGAAGGT |
| | IgG1.3f Heavy | CTCCTGCAAGGCATCTGGATACACTTTCACCAGCTACTATATGCACTGGGTGCGACAGG |
| | Chain (no C- | CCCCTGGACAAGGGCTTGAGTGGATGGGAATAATCAACCCTAGGGGTGATAGCATAATC |
| | terminal K) | TACGCACAGAAGTTCCAGGGCAGAGTCACCATGACCAGGGACACGTCCACGAGCACAGT |
| | | CTACATGGAGCTGAGCAGCCTGAGATCTGAGGACACGGCCGTGTATTACTGTGCGAGAG |
| | | ATTTCTATGGTTCGGGAAACTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 159 | 9F6 IgG1.3f | CAGGTGCAGCTGGTGGAGTCTGGGGGAGGCTTGGTCAAGCCTGGAGGGTCCCTGAGACT |
| | Heavy Chain (no | CTCCTGTGCAGCCTCTGGATTCACCTTCAGTGACTACTACATGAGCTGGATCCGCCAGG |
| | C-terminal K) | CTCCAGGGAAGGGGCTGGAGTGGGTTTCATTCATTAGTGGTGGTGGTAGTACCATATAC |
| | | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCGCG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 160 | TIM3.4 (3G4) | CAGGTGCAGCTGGTGGAGTCTGGGGGAGGCTTGGTCAAGCCTGGAGGGTCCCTGAGACT |
| | IgG1.3f Heavy | CTCCTGTGCAGCCTCTGGATTCACCTTCAGTGACTACTACATGAGCTGGATCCGCCAGG |
| | Chain (no C- | CTCCAGGGAAGGGGCTGGAGTGGGTTTCATTCATTAGTACTAGTGGTAGTATCATATAC |
| | terminal K) | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | AAGGGTATAGCAGCAGCTGGTCCTACTACTACGGTATGGACGTCTGGGGCCAAGGGACC |
| | | ACGGTCACCGTCTCCTCAGCTAGCACCAAGGGCCCATCGGTCTTCCCCCTGGCACCCTC |
| | | CTCCAAGAGCACCTCTGGGGGCACAGCGGCCCTGGGCTGCCTGGTCAAGGACTACTTCC |
| | | CCGAACCGGTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCGTGCCCTC |
| | | CAGCAGCTTGGGCACCCAGACCTACATCTGCAACGTGAATCACAAGCCCAGCAACACCA |
| | | AGGTGGACAAGAGAGTTGAGCCCAAATCTTGTGACAAAACTCACACATGCCCACCGTGC |
| | | CCAGCACCTGAAGCCGAAGGGGCCCCGTCAGTCTTCCTCTTCCCCCCAAAACCCAAGGA |
| | | CACCCTCATGATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCATAATGCCAAG |
| | | ACAAAGCCGCGGGAGGAGCAGTACAACAGCACGTACCGTGTGGTCAGCGTCCTCACCGT |
| | | CCTGCACCAGGACTGGCTGAATGGCAAGGAGTACAAGTGCAAGGTCTCCAACAAAGCCC |
| | | TCCCAGCCCCCATCGAGAAAACCATCTCCAAAGCCAAAGGGCAGCCCCGAGAACCACAG |
| | | GTGTACACCCTGCCCCCATCCCGGGAGGAGATGACCAAGAACCAGGTCAGCCTGACCTG |
| | | CCTGGTCAAAGGCTTCTATCCCAGCGACATCGCCGTGGAGTGGGAGAGCAATGGGCAGC |
| | | CGGAGAACAACTACAAGACCACGCCTCCCGTGCTGGACTCCGACGGCTCCTTCTTCCTC |
| | | TATAGCAAGCTCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCCTGTCCCCGG |
| | | GTTGA |
| 161 | TIM3.9 (17C8) | CAGGTGCAGCTGGTGGAGTCTGGGGGAGGCTTGGTCAAGCCTGGAGGGTCCCTGAGACT |
| | IgG1.3f Heavy | CTCCTGTGCAGCCTCTGGATTCACCTTCAGTGACTACTACATGAGCTGGATCCGCCAGG |
| | Chain (no C- | CTCCAGGGAAGGGGCTGGAGTGGGTTTCATTCATTAGTAGTAGTGGTAGTATCATATAC |
| | terminal K) | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | ATGGGTATAGCAGTGGCTGGGAGTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 205 | TIM3.10 (13A3) | CAGCTGCAGCTGCAGGAGTCGGGCCCAGGACTGGTGAAGCCTTCGGAGACCCTGTCCCT |
| | IgG1.1f (N60Q) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 206 | TIM3.11 (13A3) | CAGCTGCAGCTGCAGGAGTCGGGCCCAGGACTGGTGAAGCCTTCGGAGACCCTGTCCCT |
| | IgG1.1f (N60S) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACTCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 207 | TIM3.12 (13A3) | CAGCTGCAGCTGCAGGAGTCGGGCCCAGGACTGGTGAAGCCTTCGGAGACCCTGTCCCT |
| | IgG1.1f (N60A) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACGCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 208 | TIM3.13 (13A3) | CAGCTGCAGCTGCAGGAGTCGGGCCCAGGACTGGTGAAGCCTTCGGAGACCCTGTCCCT |
| | IgG1.1f (D101E) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 209 | TIM3.14 (13A3) | CAGCTGCAGCTGCAGGAGTCGGGCCCAGGACTGGTGAAGCCTTCGGAGACCCTGTCCCT |
| | IgG1.1f (P102V) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACGTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 210 | TIM3.15 (13A3) | CAGCTGCAGCTGCAGGAGTCGGGCCCAGGACTGGTGAAGCCTTCGGAGACCCTGTCCCT |
| | IgG1.1f (P102Y) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 211 | TIM3.16 (13A3) | CAGCTGCAGCTGCAGGAGTCGGGCCCAGGACTGGTGAAGCCTTCGGAGACCCTGTCCCT |
| | IgG1.1f (P102L) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 212 | TIM3.17 (13A3) | CAGCTGCAGCTGCAGGAGTCGGGCCCAGGACTGGTGAAGCCTTCGGAGACCCTGTCCCT |
| | IgG1.1f | CACCTGCACTGTCTCTGGTGGCTCCATCAGCAGTAGAAGTTACTACTGGGGCTGGATCC |
| | (N60Q/P102Y) | GCCAGCCCCCAGGGAAGGGGCTGGAGTGGATTGGGAGTATCTATTATAGTGGGTTCACC |
| | Heavy Chain | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 355 | TIM3.18 (13A3) | CAGCTGCAGCTGCAGGAGTCGGGCCCAGGACTGGTGAAGCCTTCGGAGACCCTGTCCCT |
| | IgG1.1f | CACCTGCACTGTCTCTGGTGGCTCCATCAGCAGTAGAAGTTACTACTGGGGCTGGATCC |
| | (N60Q/D101E) | GCCAGCCCCCAGGGAAGGGGCTGGAGTGGATTGGGAGTATCTATTATAGTGGGTTCACC |
| | Heavy Chain | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 213 | TIM3.8 (8B9) | CAGGTGCAGCTGCAGGAGTCGGGCCCAGGACTGGTGAAGCCTTCGGAGACCCTGTCCCT |
| | IgG1.1f (S61P) | CACCTGCACTGTCTCTGGTGGCTCCATCAGTCGTCACTACTGGAACTGGATCCGGCAGC |
| | Heavy Chain | CCCCAGGGAAGGGACTGGAGTGGATTGGGTATATCCATTACAGTGGAAGCACCAACTAC |
| | | AATCCCTCCCTCAAGAGTCGAGTCACCATATCAGTAGACACGTCCAAGAACCAGTTCTC |
| | | CCTGAAGCTGAGCTCTGTGACCGCTGCGGACACGGCCGTGTATTACTGTGCGAGAGATA |
| | | CTGGGTACTACGGTATGGACATCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAAGCAGCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTAAATGA |
| 214 | TIM3.7 (9F6) | CAGGTGCAGCTGGTGGAGTCTGGGGGAGGCTTGGTCAAGCCTGGAGGGTCCCTGAGACT |
| | IgG1.1f (A108T) | CTCCTGTGCAGCCTCTGGATTCACCTTCAGTGACTACTACATGAGCTGGATCCGCCAGG |
| | Heavy Chain | CTCCAGGGAAGGGGCTGGAGTGGGTTTCATTCATTAGTGGTGGTGGTAGTACCATATAC |
| | | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 430 | TIM3.24/14H7 | CAGGTGCACCTGGTGGAGTCTGGGGGAGGCTTGGTCAAGCCTGGAGGGTCCCTGAGACT |
| | IgG1.1f Heavy | CTCCTGTACAGCCTTCAGTGACTACTACATGAGCTGGATCCGCCAGGCTCCAGGGAAGG |
| | Chain | GGCTGGAGTGGTTATCATACATTAGTAATAGTGGTAGTATCATATACTACGCAGACTCT |
| | | GTGAAGGGCCGATTCACCATCTCCAGGGACAACGCCAAGAACTCAGTGTATCTGCAAAT |
| | | GAACAGCCTGAGAGCCGAGGACACGGCTGTGTATTACTGTGCGAGAGGGCGAATTGGTT |
| | | TTTTTGACTACTGGGGCCCGGGAACCCTGGTCACCGTCTCCTCAGCTAGCACCAAGGGC |
| | | CCATCGGTCTTCCCCCTGGCACCCTCCTCCAAGAGCACCTCTGGGGGCACAGCGGCCCT |
| | | GGGCTGCCTGGTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGACCTACATCTGCAA |
| | | CGTGAATCACAAGCCCAGCAACACCAAGGTGGACAAGAGAGTTGAGCCCAAATCTTGTG |
| | | ACAAAACTCACACATGCCCACCGTGCCCAGCACCTGAAGCCGAAGGGGCCCCGTCAGTC |
| | | TTCCTCTTCCCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACTGGTACGTGG |
| | | ACGGCGTGGAGGTGCATAATGCCAAGACAAAGCCGCGGGAGGAGCAGTACAACAGCACG |
| | | TACCGTGTGGTCAGCGTCCTCACCGTCCTGCACCAGGACTGGCTGAATGGCAAGGAGTA |
| | | CAAGTGCAAGGTCTCCAACAAAGCCCTCCCAAGCAGCATCGAGAAAACCATCTCCAAAG |
| | | CCAAAGGGCAGCCCCGAGAACCACAGGTGTACACCCTGCCCCCATCCCGGGAGGAGATG |
| | | ACCAAGAACCAGGTCAGCCTGACCTGCCTGGTCAAAGGCTTCTATCCCAGCGACATCGC |
| | | CGTGGAGTGGGAGAGCAATGGGCAGCCGGAGAACAACTACAAGACCACGCCTCCCGTGC |
| | | TGGACTCCGACGGCTCCTTCTTCCTCTATAGCAAGCTCACCGTGGACAAGAGCAGGTGG |
| | | CAGCAGGGGAACGTCTTCTCATGCTCCGTGATGCATGAGGCTCTGCACAACCACTACAC |
| | | GCAGAAGAGCCTCTCCCTGTCCCCGGGTAAATGA |
| 434 | 23B3 Ig1.f | CAGGTGCAGCTGGTGGGATCTGGGGGAGGCTTGGTCAAGCCTGGAGGGTCCCTGAGACT |
| | Heavy Chain | CTCCTGTGCAGCCTCTGGATTCACCTTCAGTGACTACTACATGAGCTGGATCCGCCAGG |
| | | CTCCAGGGAAGGGGCTGGAATGGGTTTCATTCATTAGTGGTAGTGGTAGTATCATATAC |
| | | TACGCAGACTCTGTGAAGGGCCGATTCACCATCTCCAGGGACAACGCCAAGAACTCACT |
| | | GGATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 438 | TIM3.25 (23B3) | CAGGTGCAGCTGGTGGAATCTGGGGGAGGCTTGGTCAAGCCTGGAGGGTCCCTGAGACT |
| | IgG1.1f (G6E, | CTCCTGTGCAGCCTCTGGATTCACCTTCAGTGACTACTACATGAGCTGGATCCGCCAGG |
| | D79Y) Heavy | CTCCAGGGAAGGGGCTGGAATGGGTTTCATTCATTAGTGGTAGTGGTAGTATCATATAC |
| | Chain | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 215 | TIM3.10 (13A3) | CAGCTGCAGCTGCAGGAGTCGGGCCCAGGACTGGTGAAGCCTTCGGAGACCCTGTCCCT |
| | IgG1.1f (N60Q) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 216 | TIM3.11 (13A3) | CAGCTGCAGCTGCAGGAGTCGGGCCCAGGACTGGTGAAGCCTTCGGAGACCCTGTCCCT |
| | IgG1.1f (N60S) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACTCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 217 | TIM3.12 (13A3) | CAGCTGCAGCTGCAGGAGTCGGGCCCAGGACTGGTGAAGCCTTCGGAGACCCTGTCCCT |
| | IgG1.1f (N60A) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACGCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 218 | TIM3.13 (13A3) | CAGCTGCAGCTGCAGGAGTCGGGCCCAGGACTGGTGAAGCCTTCGGAGACCCTGTCCCT |
| | IgG1.1f (D101E) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 219 | TIM3.14 (13A3) | CAGCTGCAGCTGCAGGAGTCGGGCCCAGGACTGGTGAAGCCTTCGGAGACCCTGTCCCT |
| | IgG1.1f (P102V) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACGTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 220 | TIM3.15 (13A3) | CAGCTGCAGCTGCAGGAGTCGGGCCCAGGACTGGTGAAGCCTTCGGAGACCCTGTCCCT |
| | IgG1.1f (P102Y) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 221 | TIM3.16 (13A3) | CAGCTGCAGCTGCAGGAGTCGGGCCCAGGACTGGTGAAGCCTTCGGAGACCCTGTCCCT |
| | IgG1.1f (P102L) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 222 | TIM3.17 (13A3) | CAGCTGCAGCTGCAGGAGTCGGGCCCAGGACTGGTGAAGCCTTCGGAGACCCTGTCCCT |
| | IgG1.1f | CACCTGCACTGTCTCTGGTGGCTCCATCAGCAGTAGAAGTTACTACTGGGGCTGGATCC |
| | (N60Q/P102Y) | GCCAGCCCCCAGGGAAGGGGCTGGAGTGGATTGGGAGTATCTATTATAGTGGGTTCACC |
| | Heavy Chain (no | TACTACCAACCGTCCCTCAAGAGTCGAGTCACCATATCCGTTGACACGTCCAAGAACCA |
| | C-terminal K) | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 356 | TIM3.18 (13A3) | CAGCTGCAGCTGCAGGAGTCGGGCCCAGGACTGGTGAAGCCTTCGGAGACCCTGTCCCT |
| | IgG1.1f | CACCTGCACTGTCTCTGGTGGCTCCATCAGCAGTAGAAGTTACTACTGGGGCTGGATCC |
| | (N60Q/D101E) | GCCAGCCCCCAGGGAAGGGGCTGGAGTGGATTGGGAGTATCTATTATAGTGGGTTCACC |
| | Heavy Chain (no | TACTACCAACCGTCCCTCAAGAGTCGAGTCACCATATCCGTTGACACGTCCAAGAACCA |
| | C-terminal K) | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 223 | IgG1.1f (S61P) | CAGGTGCAGCTGCAGGAGTCGGGCCCAGGACTGGTGAAGCCTTCGGAGACCCTGTCCCT |
| | TIM3.8 (8B9) | CACCTGCACTGTCTCTGGTGGCTCCATCAGTCGTCACTACTGGAACTGGATCCGGCAGC |
| | Heavy Chain (no | CCCCAGGGAAGGGACTGGAGTGGATTGGGTATATCCATTACAGTGGAAGCACCAACTAC |
| | C-terminal K) | AATCCCTCCCTCAAGAGTCGAGTCACCATATCAGTAGACACGTCCAAGAACCAGTTCTC |
| | | CCTGAAGCTGAGCTCTGTGACCGCTGCGGACACGGCCGTGTATTACTGTGCGAGAGATA |
| | | CTGGGTACTACGGTATGGACATCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAAGCAGCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTTGA |
| 224 | TIM3.7 (9F6) | CAGGTGCAGCTGGTGGAGTCTGGGGGAGGCTTGGTCAAGCCTGGAGGGTCCCTGAGACT |
| | IgG1.1f (A108T) | CTCCTGTGCAGCCTCTGGATTCACCTTCAGTGACTACTACATGAGCTGGATCCGCCAGG |
| | Heavy Chain (no | CTCCAGGGAAGGGGCTGGAGTGGGTTTCATTCATTAGTGGTGGTGGTAGTACCATATAC |
| | C-terminal K) | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 431 | TIM3.24/14H7 | CAGGTGCACCTGGTGGAGTCTGGGGGAGGCTTGGTCAAGCCTGGAGGGTCCCTGAGACT |
| | IgG1.1f Heavy | CTCCTGTACAGCCTTCAGTGACTACTACATGAGCTGGATCCGCCAGGCTCCAGGGAAGG |
| | Chai (no C- | GGCTGGAGTGGTTATCATACATTAGTAATAGTGGTAGTATCATATACTACGCAGACTCT |
| | terminal K) | GTGAAGGGCCGATTCACCATCTCCAGGGACAACGCCAAGAACTCAGTGTATCTGCAAAT |
| | | GAACAGCCTGAGAGCCGAGGACACGGCTGTGTATTACTGTGCGAGAGGGCGAATTGGTT |
| | | TTTTTGACTACTGGGGCCCGGGAACCCTGGTCACCGTCTCCTCAGCTAGCACCAAGGGC |
| | | CCATCGGTCTTCCCCCTGGCACCCTCCTCCAAGAGCACCTCTGGGGGCACAGCGGCCCT |
| | | GGGCTGCCTGGTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGACCTACATCTGCAA |
| | | CGTGAATCACAAGCCCAGCAACACCAAGGTGGACAAGAGAGTTGAGCCCAAATCTTGTG |
| | | ACAAAACTCACACATGCCCACCGTGCCCAGCACCTGAAGCCGAAGGGGCCCCGTCAGTC |
| | | TTCCTCTTCCCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACTGGTACGTGG |
| | | ACGGCGTGGAGGTGCATAATGCCAAGACAAAGCCGCGGGAGGAGCAGTACAACAGCACG |
| | | TACCGTGTGGTCAGCGTCCTCACCGTCCTGCACCAGGACTGGCTGAATGGCAAGGAGTA |
| | | CAAGTGCAAGGTCTCCAACAAAGCCCTCCCAAGCAGCATCGAGAAAACCATCTCCAAAG |
| | | CCAAAGGGCAGCCCCGAGAACCACAGGTGTACACCCTGCCCCCATCCCGGGAGGAGATG |
| | | ACCAAGAACCAGGTCAGCCTGACCTGCCTGGTCAAAGGCTTCTATCCCAGCGACATCGC |
| | | CGTGGAGTGGGAGAGCAATGGGCAGCCGGAGAACAACTACAAGACCACGCCTCCCGTGC |
| | | TGGACTCCGACGGCTCCTTCTTCCTCTATAGCAAGCTCACCGTGGACAAGAGCAGGTGG |
| | | CAGCAGGGGAACGTCTTCTCATGCTCCGTGATGCATGAGGCTCTGCACAACCACTACAC |
| | | GCAGAAGAGCCTCTCCCTGTCCCCGGGTTGA |
| 435 | 23B3 IgG1.1f | CAGGTGCAGCTGGTGGGATCTGGGGGAGGCTTGGTCAAGCCTGGAGGGTCCCTGAGACT |
| | Heavy Chain (no | CTCCTGTGCAGCCTCTGGATTCACCTTCAGTGACTACTACATGAGCTGGATCCGCCAGG |
| | C-terminal K) | CTCCAGGGAAGGGGCTGGAATGGGTTTCATTCATTAGTGGTAGTGGTAGTATCATATAC |
| | | TACGCAGACTCTGTGAAGGGCCGATTCACCATCTCCAGGGACAACGCCAAGAACTCACT |
| | | GGATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 439 | TIM3.25 (23B3) | CAGGTGCAGCTGGTGGAATCTGGGGGAGGCTTGGTCAAGCCTGGAGGGTCCCTGAGACT |
| | IgG1.1f (G6E, | CTCCTGTGCAGCCTCTGGATTCACCTTCAGTGACTACTACATGAGCTGGATCCGCCAGG |
| | D79Y) Heavy | CTCCAGGGAAGGGGCTGGAATGGGTTTCATTCATTAGTGGTAGTGGTAGTATCATATAC |
| | Chain (no C- | TACGCAGACTCTGTGAAGGGCCGATTCACCATCTCCAGGGACAACGCCAAGAACTCACT |
| | terminal K) | GTATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAAGCAGCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 225 | TIM3.10 (13A3) | CAGCTGCAGCTGCAGGAGTCGGGCCCAGGACTGGTGAAGCCTTCGGAGACCCTGTCCCT |
| | IgG1.3f (N60Q) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 226 | TIM3.11 (13A3) | CAGCTGCAGCTGCAGGAGTCGGGCCCAGGACTGGTGAAGCCTTCGGAGACCCTGTCCCT |
| | IgG1.3f (N60S) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACTCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 227 | TIM3.12 (13A3) | CAGCTGCAGCTGCAGGAGTCGGGCCCAGGACTGGTGAAGCCTTCGGAGACCCTGTCCCT |
| | IgG1.3f (N60A) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACGCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 228 | TIM3.13 (13A3) | CAGCTGCAGCTGCAGGAGTCGGGCCCAGGACTGGTGAAGCCTTCGGAGACCCTGTCCCT |
| | IgG1.3f (D101E) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 229 | TIM3.14 (13A3) | CAGCTGCAGCTGCAGGAGTCGGGCCCAGGACTGGTGAAGCCTTCGGAGACCCTGTCCCT |
| | IgG1.3f (P102V) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACGTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 230 | TIM3.15 (13A3) | CAGCTGCAGCTGCAGGAGTCGGGCCCAGGACTGGTGAAGCCTTCGGAGACCCTGTCCCT |
| | IgG1.3f (P102Y) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 231 | TIM3.16 (13A3) | CAGCTGCAGCTGCAGGAGTCGGGCCCAGGACTGGTGAAGCCTTCGGAGACCCTGTCCCT |
| | IgG1.3f (P102L) | CACCTGCACTGTCTCTGGTGGCTCCATCAGCAGTAGAAGTTACTACTGGGGCTGGATCC |
| | Heavy Chain | GCCAGCCCCCAGGGAAGGGGCTGGAGTGGATTGGGAGTATCTATTATAGTGGGTTCACC |
| | | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 232 | TIM3.17 (13A3) | CAGCTGCAGCTGCAGGAGTCGGGCCCAGGACTGGTGAAGCCTTCGGAGACCCTGTCCCT |
| | IgG1.3f | CACCTGCACTGTCTCTGGTGGCTCCATCAGCAGTAGAAGTTACTACTGGGGCTGGATCC |
| | (N60Q/P102Y) | GCCAGCCCCCAGGGAAGGGGCTGGAGTGGATTGGGAGTATCTATTATAGTGGGTTCACC |
| | Heavy Chain | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 357 | TIM3.18 (13A3) | CAGCTGCAGCTGCAGGAGTCGGGCCCAGGACTGGTGAAGCCTTCGGAGACCCTGTCCCT |
| | IgG1.3f | CACCTGCACTGTCTCTGGTGGCTCCATCAGCAGTAGAAGTTACTACTGGGGCTGGATCC |
| | (N60Q/D101E) | GCCAGCCCCCAGGGAAGGGGCTGGAGTGGATTGGGAGTATCTATTATAGTGGGTTCACC |
| | Heavy Chain | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 233 | TIM3.8 (8B9) | CAGGTGCAGCTGCAGGAGTCGGGCCCAGGACTGGTGAAGCCTTCGGAGACCCTGTCCCT |
| | IgG1.3f (S61P) | CACCTGCACTGTCTCTGGTGGCTCCATCAGTCGTCACTACTGGAACTGGATCCGGCAGC |
| | Heavy Chain | CCCCAGGGAAGGGACTGGAGTGGATTGGGTATATCCATTACAGTGGAAGCACCAACTAC |
| | | AATCCCTCCCTCAAGAGTCGAGTCACCATATCAGTAGACACGTCCAAGAACCAGTTCTC |
| | | CCTGAAGCTGAGCTCTGTGACCGCTGCGGACACGGCCGTGTATTACTGTGCGAGAGATA |
| | | CTGGGTACTACGGTATGGACATCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAGCCCCCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTAAATGA |
| 234 | TIM3.7 (9F6) | CAGGTGCAGCTGGTGGAGTCTGGGGGAGGCTTGGTCAAGCCTGGAGGGTCCCTGAGACT |
| | IgG1.3f (A108T) | CTCCTGTGCAGCCTCTGGATTCACCTTCAGTGACTACTACATGAGCTGGATCCGCCAGG |
| | Heavy Chain | CTCCAGGGAAGGGGCTGGAGTGGGTTTCATTCATTAGTGGTGGTGGTAGTACCATATAC |
| | | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 432 | TIM3.24/14H7 | CAGGTGCACCTGGTGGAGTCTGGGGGAGGCTTGGTCAAGCCTGGAGGGTCCCTGAGACT |
| | IgG1.3f Heavy | CTCCTGTACAGCCTTCAGTGACTACTACATGAGCTGGATCCGCCAGGCTCCAGGGAAGG |
| | Chain | GGCTGGAGTGGTTATCATACATTAGTAATAGTGGTAGTATCATATACTACGCAGACTCT |
| | | GTGAAGGGCCGATTCACCATCTCCAGGGACAACGCCAAGAACTCAGTGTATCTGCAAAT |
| | | GAACAGCCTGAGAGCCGAGGACACGGCTGTGTATTACTGTGCGAGAGGGCGAATTGGTT |
| | | TTTTTGACTACTGGGGCCCGGGAACCCTGGTCACCGTCTCCTCAGCTAGCACCAAGGGC |
| | | CCATCGGTCTTCCCCCTGGCACCCTCCTCCAAGAGCACCTCTGGGGGCACAGCGGCCCT |
| | | GGGCTGCCTGGTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGACCTACATCTGCAA |
| | | CGTGAATCACAAGCCCAGCAACACCAAGGTGGACAAGAGAGTTGAGCCCAAATCTTGTG |
| | | ACAAAACTCACACATGCCCACCGTGCCCAGCACCTGAAGCCGAAGGGGCCCCGTCAGTC |
| | | TTCCTCTTCCCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACTGGTACGTGG |
| | | ACGGCGTGGAGGTGCATAATGCCAAGACAAAGCCGCGGGAGGAGCAGTACAACAGCACG |
| | | TACCGTGTGGTCAGCGTCCTCACCGTCCTGCACCAGGACTGGCTGAATGGCAAGGAGTA |
| | | CAAGTGCAAGGTCTCCAACAAAGCCCTCCCAGCCCCCATCGAGAAAACCATCTCCAAAG |
| | | CCAAAGGGCAGCCCCGAGAACCACAGGTGTACACCCTGCCCCCATCCCGGGAGGAGATG |
| | | ACCAAGAACCAGGTCAGCCTGACCTGCCTGGTCAAAGGCTTCTATCCCAGCGACATCGC |
| | | CGTGGAGTGGGAGAGCAATGGGCAGCCGGAGAACAACTACAAGACCACGCCTCCCGTGC |
| | | TGGACTCCGACGGCTCCTTCTTCCTCTATAGCAAGCTCACCGTGGACAAGAGCAGGTGG |
| | | CAGCAGGGGAACGTCTTCTCATGCTCCGTGATGCATGAGGCTCTGCACAACCACTACAC |
| | | GCAGAAGAGCCTCTCCCTGTCCCCGGGTAAATGA |
| 436 | 23B3 IgG1.3f | CAGGTGCAGCTGGTGGGATCTGGGGGAGGCTTGGTCAAGCCTGGAGGGTCCCTGAGACT |
| | Heavy Chain | CTCCTGTGCAGCCTCTGGATTCACCTTCAGTGACTACTACATGAGCTGGATCCGCCAGG |
| | | CTCCAGGGAAGGGGCTGGAATGGGTTTCATTCATTAGTGGTAGTGGTAGTATCATATAC |
| | | TACGCAGACTCTGTGAAGGGCCGATTCACCATCTCCAGGGACAACGCCAAGAACTCACT |
| | | GGATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 440 | TIM3.25 (23B3) | CAGGTGCAGCTGGTGGAATCTGGGGGAGGCTTGGTCAAGCCTGGAGGGTCCCTGAGACT |
| | IgG1.3f (G6E, | CTCCTGTGCAGCCTCTGGATTCACCTTCAGTGACTACTACATGAGCTGGATCCGCCAGG |
| | D79Y) Heavy | CTCCAGGGAAGGGGCTGGAATGGGTTTCATTCATTAGTGGTAGTGGTAGTATCATATAC |
| | Chain | TACGCAGACTCTGTGAAGGGCCGATTCACCATCTCCAGGGACAACGCCAAGAACTCACT |
| | | GTATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTA |
| | | AATGA |
| 235 | TIM3.10 (13A3) | CAGCTGCAGCTGCAGGAGTCGGGCCCAGGACTGGTGAAGCCTTCGGAGACCCTGTCCCT |
| | IgG1.3f (N60Q) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACCAACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 236 | TIM3.11 (13A3) | CAGCTGCAGCTGCAGGAGTCGGGCCCAGGACTGGTGAAGCCTTCGGAGACCCTGTCCCT |
| | IgG1.3f (N60S) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACTCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 237 | TIM3.12 (13A3) | CAGCTGCAGCTGCAGGAGTCGGGCCCAGGACTGGTGAAGCCTTCGGAGACCCTGTCCCT |
| | IgG1.3f (N60A) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACGCACCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 238 | TIM3.13 (13A3) | CAGCTGCAGCTGCAGGAGTCGGGCCCAGGACTGGTGAAGCCTTCGGAGACCCTGTCCCT |
| | IgG1.3f (D101E) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 239 | TIM3.14 (13A3) | CAGCTGCAGCTGCAGGAGTCGGGCCCAGGACTGGTGAAGCCTTCGGAGACCCTGTCCCT |
| | IgG1.3f (P102V) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACGTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 240 | TIM3.15 (13A3) | CAGCTGCAGCTGCAGGAGTCGGGCCCAGGACTGGTGAAGCCTTCGGAGACCCTGTCCCT |
| | IgG1.3f (P102Y) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 241 | TIM3.16 (13A3) | CAGCTGCAGCTGCAGGAGTCGGGCCCAGGACTGGTGAAGCCTTCGGAGACCCTGTCCCT |
| | IgG1.3f (P102L) | CACCTGCACTGTCTCTGGTGGCTCCATCAGCAGTAGAAGTTACTACTGGGGCTGGATCC |
| | Heavy Chain (no | GCCAGCCCCCAGGGAAGGGGCTGGAGTGGATTGGGAGTATCTATTATAGTGGGTTCACC |
| | C-terminal K) | TACTACAACCCGTCCCTCAAGAGTCGAGTCACCATATCCGTTGACACGTCCAAGAACCA |
| | | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACCTATGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 242 | TIM3.17 (13A3) | CAGCTGCAGCTGCAGGAGTCGGGCCCAGGACTGGTGAAGCCTTCGGAGACCCTGTCCCT |
| | IgG1.3f | CACCTGCACTGTCTCTGGTGGCTCCATCAGCAGTAGAAGTTACTACTGGGGCTGGATCC |
| | (N60Q/P102Y) | GCCAGCCCCCAGGGAAGGGGCTGGAGTGGATTGGGAGTATCTATTATAGTGGGTTCACC |
| | Heavy Chain (no | TACTACCAACCGTCCCTCAAGAGTCGAGTCACCATATCCGTTGACACGTCCAAGAACCA |
| | C-terminal K) | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGACTACTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 358 | TIM3.18 (13A3) | CAGCTGCAGCTGCAGGAGTCGGGCCCAGGACTGGTGAAGCCTTCGGAGACCCTGTCCCT |
| | IgG1.3f | CACCTGCACTGTCTCTGGTGGCTCCATCAGCAGTAGAAGTTACTACTGGGGCTGGATCC |
| | (N60Q/D101E) | GCCAGCCCCCAGGGAAGGGGCTGGAGTGGATTGGGAGTATCTATTATAGTGGGTTCACC |
| | Heavy Chain (no | TACTACCAACCGTCCCTCAAGAGTCGAGTCACCATATCCGTTGACACGTCCAAGAACCA |
| | C-terminal K) | GTTCTCCCTGAAGCTGAGCTCTGTGACCGCCGCAGACACGGCTGTGTATTATTGTGCGA |
| | | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 374 | TIM3.18 (13A3) | CAGCTGCAGCTGCAGGAGTCGGGCCCAGGACTGGTGAAGCCTTCGGAGACCCTGTCCCT |
| | IgG1.3f | CACCTGCACTGTCTCTGGTGGCTCCATCAGCAGTAGAAGTTACTACTGGGGCTGGATTC |
| | (N60Q/D101E) | GCCAGCCCCCAGGGAAGGGGCTGGAGTGGATTGGGAGTATCTATTATAGTGGGTTCACC |
| | (T168C) Heavy | TACTACCAACCGTCCCTCAAGAGTCGAGTCACCATATCCGTTGACACGTCCAAGAACCA |
| | Chain (no | GTTCTCCCTGAAGCTGAGCTCTGTGACCGCCGCAGACACGGCTGTGTATTATTGTGCGA |
| | C-terminal K) | CAGGGGGGCCCTACGGTGACTACGCCCACTGGTTCGAACCCTGGGGCCAGGGAACCCTG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 243 | TIM3.8 (8B9) | CAGGTGCAGCTGCAGGAGTCGGGCCCAGGACTGGTGAAGCCTTCGGAGACCCTGTCCCT |
| | IgG1.3f (S61P) | CACCTGCACTGTCTCTGGTGGCTCCATCAGTCGTCACTACTGGAACTGGATCCGGCAGC |
| | Heavy Chain (no | CCCCAGGGAAGGGACTGGAGTGGATTGGGTATATCCATTACAGTGGAAGCACCAACTAC |
| | C-terminal K) | AATCCCTCCCTCAAGAGTCGAGTCACCATATCAGTAGACACGTCCAAGAACCAGTTCTC |
| | | CCTGAAGCTGAGCTCTGTGACCGCTGCGGACACGGCCGTGTATTACTGTGCGAGAGATA |
| | | CTGGGTACTACGGTATGGACATCTGGGGCCAAGGGACCACGGTCACCGTCTCCTCAGCT |
| | | AGCACCAAGGGCCCATCGGTCTTCCCCCTGGCACCCTCCTCCAAGAGCACCTCTGGGGG |
| | | CACAGCGGCCCTGGGCTGCCTGGTCAAGGACTACTTCCCCGAACCGGTGACGGTGTCGT |
| | | GGAACTCAGGCGCCCTGACCAGCGGCGTGCACACCTTCCCGGCTGTCCTACAGTCCTCA |
| | | GGACTCTACTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAGAGTTGAGC |
| | | CCAAATCTTGTGACAAAACTCACACATGCCCACCGTGCCCAGCACCTGAAGCCGAAGGG |
| | | GCCCCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGACTGGCTGAA |
| | | TGGCAAGGAGTACAAGTGCAAGGTCTCCAACAAAGCCCTCCCAGCCCCCATCGAGAAAA |
| | | CCATCTCCAAAGCCAAAGGGCAGCCCCGAGAACCACAGGTGTACACCCTGCCCCCATCC |
| | | CGGGAGGAGATGACCAAGAACCAGGTCAGCCTGACCTGCCTGGTCAAAGGCTTCTATCC |
| | | CAGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAGGCTCTGCA |
| | | CAACCACTACACGCAGAAGAGCCTCTCCCTGTCCCCGGGTTGA |
| 244 | TIM3.7 (9F6) | CAGGTGCAGCTGGTGGAGTCTGGGGGAGGCTTGGTCAAGCCTGGAGGGTCCCTGAGACT |
| | IgG1.3f (A108T) | CTCCTGTGCAGCCTCTGGATTCACCTTCAGTGACTACTACATGAGCTGGATCCGCCAGG |
| | Heavy Chain (no | CTCCAGGGAAGGGGCTGGAGTGGGTTTCATTCATTAGTGGTGGTGGTAGTACCATATAC |
| | C-terminal K) | TACGCAGACTCTGTGAAGGGCCGATTCACCATCTCCAGGGACAACGCCAAGAACTCGCT |
| | | GTTTCTGCAAATGAACAGCCTGAGAGTCGAGGACACGGCTGTGTATTACTGTGCGAGAG |
| | | ATGGCTATAGCAGTGGCTGGTACTACTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 433 | TIM3.24/14H7 | CAGGTGCACCTGGTGGAGTCTGGGGGAGGCTTGGTCAAGCCTGGAGGGTCCCTGAGACT |
| | IgG1.3f Heavy | CTCCTGTACAGCCTTCAGTGACTACTACATGAGCTGGATCCGCCAGGCTCCAGGGAAGG |
| | Chain (no C- | GGCTGGAGTGGTTATCATACATTAGTAATAGTGGTAGTATCATATACTACGCAGACTCT |
| | terminal K) | GTGAAGGGCCGATTCACCATCTCCAGGGACAACGCCAAGAACTCAGTGTATCTGCAAAT |
| | | GAACAGCCTGAGAGCCGAGGACACGGCTGTGTATTACTGTGCGAGAGGGCGAATTGGTT |
| | | TTTTTGACTACTGGGGCCCGGGAACCCTGGTCACCGTCTCCTCAGCTAGCACCAAGGGC |
| | | CCATCGGTCTTCCCCCTGGCACCCTCCTCCAAGAGCACCTCTGGGGGCACAGCGGCCCT |
| | | GGGCTGCCTGGTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGACCTACATCTGCAA |
| | | CGTGAATCACAAGCCCAGCAACACCAAGGTGGACAAGAGAGTTGAGCCCAAATCTTGTG |
| | | ACAAAACTCACACATGCCCACCGTGCCCAGCACCTGAAGCCGAAGGGGCCCCGTCAGTC |
| | | TTCCTCTTCCCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACTGGTACGTGG |
| | | ACGGCGTGGAGGTGCATAATGCCAAGACAAAGCCGCGGGAGGAGCAGTACAACAGCACG |
| | | TACCGTGTGGTCAGCGTCCTCACCGTCCTGCACCAGGACTGGCTGAATGGCAAGGAGTA |
| | | CAAGTGCAAGGTCTCCAACAAAGCCCTCCCAGCCCCCATCGAGAAAACCATCTCCAAAG |
| | | CCAAAGGGCAGCCCCGAGAACCACAGGTGTACACCCTGCCCCCATCCCGGGAGGAGATG |
| | | ACCAAGAACCAGGTCAGCCTGACCTGCCTGGTCAAAGGCTTCTATCCCAGCGACATCGC |
| | | CGTGGAGTGGGAGAGCAATGGGCAGCCGGAGAACAACTACAAGACCACGCCTCCCGTGC |
| | | TGGACTCCGACGGCTCCTTCTTCCTCTATAGCAAGCTCACCGTGGACAAGAGCAGGTGG |
| | | CAGCAGGGGAACGTCTTCTCATGCTCCGTGATGCATGAGGCTCTGCACAACCACTACAC |
| | | GCAGAAGAGCCTCTCCCTGTCCCCGGGTTGA |
| 437 | 23B3 IgG1.3f | CAGGTGCAGCTGGTGGGATCTGGGGGAGGCTTGGTCAAGCCTGGAGGGTCCCTGAGACT |
| | Heavy Chain (no | CTCCTGTGCAGCCTCTGGATTCACCTTCAGTGACTACTACATGAGCTGGATCCGCCAGG |
| | C-terminal K) | CTCCAGGGAAGGGGCTGGAATGGGTTTCATTCATTAGTGGTAGTGGTAGTATCATATAC |
| | | TACGCAGACTCTGTGAAGGGCCGATTCACCATCTCCAGGGACAACGCCAAGAACTCACT |
| | | GGATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 441 | TIM3.25 (23B3) | CAGGTGCAGCTGGTGGAATCTGGGGGAGGCTTGGTCAAGCCTGGAGGGTCCCTGAGACT |
| | IgG1.3f (G6E, | CTCCTGTGCAGCCTCTGGATTCACCTTCAGTGACTACTACATGAGCTGGATCCGCCAGG |
| | D79Y) Heavy | CTCCAGGGAAGGGGCTGGAATGGGTTTCATTCATTAGTGGTAGTGGTAGTATCATATAC |
| | Chain (no C- | TACGCAGACTCTGTGAAGGGCCGATTCACCATCTCCAGGGACAACGCCAAGAACTCACT |
| | terminal K) | GTATCTGCAAATGAACAGCCTGAGAGCCGAGGACACGGCTGTGTATTACTGTGCGAGAG |
| | | ACGGTATGGTTCGGGGAATGAACTTCTACGGTATGGACGTCTGGGGCCAAGGGACCACG |
| | | GTCACCGTCTCCTCAGCTAGCACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCG |
| | | GCTGTCCTACAGTCCTCAGGACTCTACTCCCTCAGCAGCGTGGTGACCGTGCCCTCCAG |
| | | CAGCTTGGGCACCCAGACCTACATCTGCAACGTGAATCACAAGCCCAGCAACACCAAGG |
| | | TGGACAAGAGAGTTGAGCCCAAATCTTGTGACAAAACTCACACATGCCCACCGTGCCCA |
| | | GCACCTGAAGCCGAAGGGGCCCCGTCAGTCTTCCTCTTCCCCCCAAAACCCAAGGACAC |
| | | CCTCATGATCTCCCGGACCCCTGAGGTCACATGCGTGGTGGTGGACGTGAGCCACGAAG |
| | | ACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCC |
| | | CAGCCCCCATCGAGAAAACCATCTCCAAAGCCAAAGGGCAGCCCCGAGAACCACAGGTG |
| | | TACACCCTGCCCCCATCCCGGGAGGAGATGACCAAGAACCAGGTCAGCCTGACCTGCCT |
| | | GGTCAAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTCTAT |
| | | AGCAAGCTCACCGTGGACAAGAGCAGGTGGCAGCAGGGGAACGTCTTCTCATGCTCCGT |
| | | GATGCATGAGGCTCTGCACAACCACTACACGCAGAAGAGCCTCTCCCTGTCCCCGGGTT |
| | | GA |
| 162 | TIM3.5 (13A3), | GAAATTGTGTTGACGCAGTCTCCAGGCACCCTGTCTTTGTCTCCAGGGGAAAGAGCCAC |
| | TIM3.2 (17C3), | CCTCTCCTGCAGGGCCAGTCAGAGTGTTAGCAGCAGCTACTTAGCCTGGTACCAGCAGA |
| | TIM3.4 (3G4), | AACCTGGCCAGGCTCCCAGGCTCCTCATCTATGGTGCATCCAGCAGGGCCACTGGCATC |
| | TIM3.25 IgG1 | CCAGACAGGTTCAGTGGCAGTGGGTCTGGGACAGACTTCACTCTCACCATCAGCAGACT |
| | Light Chain | GGAGCCTGAAGATTTTGCAGTGTATTACTGTCAGCAGTATGGTAGCTCACCGATCACCT |
| | | TCGGCCAAGGGACACGACTGGAGATTAAACGTACGGTGGCTGCACCATCTGTCTTCATC |
| | | TTCCCGCCATCTGATGAGCAGTTGAAATCTGGAACTGCCTCTGTTGTGTGCCTGCTGAA |
| | | TAACTTCTATCCCAGAGAGGCCAAAGTACAGTGGAAGGTGGATAACGCCCTCCAATCGG |
| | | GTAACTCCCAGGAGAGTGTCACAGAGCAGGACAGCAAGGACAGCACCTACAGCCTCAGC |
| | | AGCACCCTGACGCTGAGCAAAGCAGACTACGAGAAACACAAAGTCTACGCCTGCGAAGT |
| | | CACCCATCAGGGCCTGAGCTCGCCCGTCACAAAGAGCTTCAACAGGGGAGAGTGTTAG |
| 163 | 8B9, TIM3.6 | GAAATTGTGTTGACGCAGTCTCCAGGCACCCTGTCTTTGTCTCCAGGGGAAAGAGCCAC |
| | (8C4), TIM3.9 | CCTCTCCTGCAGGGCCAGTCAGAGTGTTAGCAGCAGCTACTTAGCCTGGTACCAGCAGA |
| | (17C8) IgG1 | AACCTGGCCAGGCTCCCAGGCTCCTCATCTATGGTGCATCCAGCAGGGCCACTGGCATC |
| | Light Chain | CCAGACAGGTTCAGTGGCAGTGGGTCTGGGACAGACTTCACTCTCACCATCAGCAGACT |
| | | GGAGCCTGAAGATTTTGCAGTGTATTACTGTCAGCAGTATGGTAGCTCACCTCTCACTT |
| | | TCGGCGGAGGGACCAAGGTGGAGATCAAACGTACGGTGGCTGCACCATCTGTCTTCATC |
| | | TTCCCGCCATCTGATGAGCAGTTGAAATCTGGAACTGCCTCTGTTGTGTGCCTGCTGAA |
| | | TAACTTCTATCCCAGAGAGGCCAAAGTACAGTGGAAGGTGGATAACGCCCTCCAATCGG |
| | | GTAACTCCCAGGAGAGTGTCACAGAGCAGGACAGCAAGGACAGCACCTACAGCCTCAGC |
| | | AGCACCCTGACGCTGAGCAAAGCAGACTACGAGAAACACAAAGTCTACGCCTGCGAAGT |
| | | CACCCATCAGGGCCTGAGCTCGCCCGTCACAAAGAGCTTCAACAGGGGAGAGTGTTAG |
| 165 | 9F6 VK1 IgG1 | GCCATCCAGTTGACCCAGTCTCCATCCTCCCTGTCTGCATCTGTAGGAGACAGAGTCAC |
| | Light Chain | CATCACTTGCCGGGCAAGTCAGGGCATTAGCAGTGCTTTAGCCTGGTATCAGCAGAAAC |
| | | CAGGGAAAGCTCCTAAGCTCCTGATCTATGATGCCTCCAGTTTGGAAAGTGGGGTCCCA |
| | | TCAAGGTTCAGCGGCAGTGGATCTGGGACAGATTTCACTCTCACCATCAGCAGCCTGCA |
| | | GCCTGAAGATTTTGCAACTTATTACTGTCAACAGTTTAATAGTTACCCTCGGACGTTCG |
| | | GCCAAGGGACCAAGGTGGAAATCAAACGTACGGTGGCTGCACCATCTGTCTTCATCTTC |
| | | CCGCCATCTGATGAGCAGTTGAAATCTGGAACTGCCTCTGTTGTGTGCCTGCTGAATAA |
| | | CTTCTATCCCAGAGAGGCCAAAGTACAGTGGAAGGTGGATAACGCCCTCCAATCGGGTA |
| | | ACTCCCAGGAGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGCGAAGTCAC |
| | | CCATCAGGGCCTGAGCTCGCCCGTCACAAAGAGCTTCAACAGGGGAGAGTGTTAG |
| 166 | 9F6 VK2 IgG1 | GAAATTGTGTTGACGCAGTCTCCAGGCACCCTGTCTTTGTCTCCAGGGGAAAGAGCCAC |
| | Light Chain | CCTCTCCTGCAGGGCCAGTCAGAGTGTTAGCAGCAGCTACTTAGCCTGGTACCAGCAGA |
| | | AACCTGGCCAGGCTCCCAGGCTCCTCATCTATGGTGCATCCAGCAGGGCCACTGGCATC |
| | | CCAGACAGGTTCAGTGGCAGTGGGTCTGGGACAGACTTCACTCTCACCATCAGCAGACT |
| | | GGAGCCTGAAGATTTTGCAGTGTATTACTGTCAGCAGTATGGTAGCTCACTCACTTTCG |
| | | GCGGAGGGACCAAGGTGGAGATCAAACGTACGGTGGCTGCACCATCTGTCTTCATCTTC |
| | | CCGCCATCTGATGAGCAGTTGAAATCTGGAACTGCCTCTGTTGTGTGCCTGCTGAATAA |
| | | CTTCTATCCCAGAGAGGCCAAAGTACAGTGGAAGGTGGATAACGCCCTCCAATCGGGTA |
| | | ACTCCCAGGAGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGCGAAGTCAC |
| | | CCATCAGGGCCTGAGCTCGCCCGTCACAAAGAGCTTCAACAGGGGAGAGTGTTAG |
| 164 | 9F6 VK3 IgG1 | GAAATTGTGTTGACGCAGTCTCCAGGCACCCTGTCTTTGTCTCCAGGGGAAAGAGCCAC |
| | Light Chain | CCTCTCCTGCAGGGCCAGTCAGAGTGTTAGCAGCAGCTACTTAGCCTGGTACCAGCAGA |
| | | AACCTGGCCAGGCTCCCAGGCTCCTCATCTATGGTGCATCCAGCAGGGCCACTGGCATC |
| | | CCAGACAGGTTCAGTGGCAGTGGGTCTGGGACAGACTTCACTCTCACCATCAGCAGACT |
| | | GGAGCCTGAAGATTTTGCAGTGTATTACTGTCAGCAGTATGGTAGCTCACCGCTCACTT |
| | | TCGGCGGAGGGACCAAGGTGGAGATCAAACGTACGGTGGCTGCACCATCTGTCTTCATC |
| | | TTCCCGCCATCTGATGAGCAGTTGAAATCTGGAACTGCCTCTGTTGTGTGCCTGCTGAA |
| | | TAACTTCTATCCCAGAGAGGCCAAAGTACAGTGGAAGGTGGATAACGCCCTCCAATCGG |
| | | GTAACTCCCAGGAGAGTGTCACAGAGCAGGACAGCAAGGACAGCACCTACAGCCTCAGC |
| | | AGCACCCTGACGCTGAGCAAAGCAGACTACGAGAAACACAAAGTCTACGCCTGCGAAGT |
| | | CACCCATCAGGGCCTGAGCTCGCCCGTCACAAAGAGCTTCAACAGGGGAGAGTGTTAG |
| 442 | TIM3.24 (14H7) | GAAATTGTGTTGACGCAGTCTCCAGGCACCCTGTCTTTGTCTCCAGGGGAAAGAGCCAC |
| | IgG1 Light | CCTCTCCTGCAGGGCCAGTCAGAGTGTTAGCAGCAGCTACTTAGCCTGGTACCAGCAGA |
| | Chain | AACCTGGCCAGGCTCCCAGGCTCCTCATCTATGGTGCATCCAGCAGGGCCACTGGCATC |
| | | CCAGACAGGTTCAGTGGCAGTGGGTCTGGGACAGACTTCACTCTCACCATCAGCAGACT |
| | | GGAGCCTGAAGATTTTGCAGTGTATTACTGTCAGCAGTATGGTAGCTCACCTCTCACTT |
| | | TCGGCCCTGGGACCAAAGTGGATATCAAACGAACTGTGGCTGCACCATCTGTCTTCATC |
| | | TTCCCGCCATCTGATGAGCAGTTGAAATCTGGAACTGCCTCTGTTGTGTGCCTGCTGAA |
| | | TAACTTCTATCCCAGAGAGGCCAAAGTACAGTGGAAGGTGGATAACGCCCTCCAATCGG |
| | | GTAACTCCCAGGAGAGTGTCACAGAGCAGGACAGCAAGGACAGCACCTACAGCCTCAGC |
| | | AGCACCCTGACGCTGAGCAAAGCAGACTACGAGAAACACAAAGTCTACGCCTGCGAAGT |
| | | CACCCATCAGGGCCTGAGCTCGCCCGTCACAAAGAGCTTCAACAGGGGAGAGTGT |
| 443 | 23B3 IgG1 Light | GAAATTGTGTTGACGCAGTCTCCAGGCACCCTGTCTTTGTCTCCAGGGGAAAGAGCCAC |
| | Chain (VK1) | CCTCTCCTGCAGGGCCAGTCAGAGTGTTAGCAGCAGCTACTTAGCCTGGTACCAGCAGA |
| | | AACCTGGCCAGGCTCCCAGGCTCCTCATCTATGGTGCATCCAGCAGGGCCACTGGCATC |
| | | CCAGACAGGTTCAGTGGCAGTGGGTCTGGGACAGACTTCACTCTCACCATCAGCAGACT |
| | | GGAGCCTGAAGATTTTGCAGTGTATTACTGTCAGCAGTATGGTAGCTCACCGATCACCT |
| | | TCGGCCAAGGGACACGACTGGAGATTAAACGAACTGTGGCTGCACCATCTGTCTTCATC |
| | | TTCCCGCCATCTGATGAGCAGTTGAAATCTGGAACTGCCTCTGTTGTGTGCCTGCTGAA |
| | | TAACTTCTATCCCAGAGAGGCCAAAGTACAGTGGAAGGTGGATAACGCCCTCCAATCGG |
| | | GTAACTCCCAGGAGAGTGTCACAGAGCAGGACAGCAAGGACAGCACCTACAGCCTCAGC |
| | | AGCACCCTGACGCTGAGCAAAGCAGACTACGAGAAACACAAAGTCTACGCCTGCGAAGT |
| | | CACCCATCAGGGCCTGAGCTCGCCCGTCACAAAGAGCTTCAACAGGGGAGAGTGT |
| 444 | 23B3 IgG1 Light | GAAATTGTGTTGACGCAGTCTCCAGGCACCCTGTCTTTGTCTCCAGGGGAAAGAGCCAC |
| | Chain (VK2) | CCTCTCCTGCAGGGCCAGTCAGAGTGTTAGCAGCAGCTACTTAGCCTGGTACCAGCAGA |
| | | AACCTGGCCAGGCTCCCAGGCTCCTCATCTATGGTGCATCCAGCAGGGCCACTGGCATC |
| | | CCAGACAGGTTCAGTGGCAGTGGGTCTGGGACAGACTTCACTCTCACCATCAGCAGACT |
| | | GGAGCCTGAAGATTTTGCAGTGTATTACTGTCAGCAGTATGGTAGCTCACCTTTCGGCG |
| | | GAGGGACCAAGGTGGAGATCAAACGAACTGTGGCTGCACCATCTGTCTTCATCTTCCCG |
| | | CCATCTGATGAGCAGTTGAAATCTGGAACTGCCTCTGTTGTGTGCCTGCTGAATAACTT |
| | | CTATCCCAGAGAGGCCAAAGTACAGTGGAAGGTGGATAACGCCCTCCAATCGGGTAACT |
| | | CCCAGGAGAGTGTCACAGAGCAGGACAGCAAGGACAGCACCTACAGCCTCAGCAGCACC |
| | | CTGACGCTGAGCAAAGCAGACTACGAGAAACACAAAGTCTACGCCTGCGAAGTCACCCA |
| | | TCAGGGCCTGAGCTCGCCCGTCACAAAGAGCTTCAACAGGGGAGAGTGT |

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); ); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1 Anti-TIM3 Antibody Monotherapy

A clinical study of a TIM3 antibody, administered as a single agent, will be performed in patients with selected advanced solid tumors. The patient progression after the administration will be assessed.

### Example 2 Anti-TIM3 Antibody Combination Therapy

A clinical study of a TIM3 antibody, administered in combination with an anti-PD1 antibody, e.g., nivolumab, will be performed in patients with selected advanced solid tumors. The patient progression after the administration will be assessed.

### Example 3 Anti-TIM3 Antibody Combination Therapy with Nivolumab

A phase 1/phase 2 clinical study of a TIM3 antibody, administered in combination with an anti-PD1 antibody, e.g., nivolumab, is ongoing in patients with selected advanced solid tumors. The purpose of this study is to determine whether an anti-TIM3 antibody both by itself and in combination with an anti-PD-1 antibody (e.g., nivolumab) is safe and tolerable in the treatment of advanced malignant tumors.

The primary outcome measures of this unmasked, non-randomized study include: the incidence of adverse events (AEs), the incidence of serious adverse events (SAEs), the incidence of AEs leading to discontinuation and deaths, and the incidence of AEs meeting protocol defined dose-limiting toxicities (DLTs) criteria. Primary outcome measures will be monitored over approximately 2 years.

Secondary outcome measures include objective response rate (ORR), median duration of response (mDOR), progression free survival rate (PFSR), maximum observed serum concentration (Cₘₐₓ), time of maximum observed serum concentration (Tₘₐₓ), area under the serum concentration-time curve from time zero to time of last quantifiable concentration [AUC(0-T)], observed concentration at the end of a dosing interval (Cₜₐᵤ), area under the serum concentration-time curve in one dosing interval [AUC(TAU)], trough observed serum concentration at the end of the dosing interval (C_{trough}), concentration at the end of infusion (Cₑₒᵢ), and incidence of anti-drug antibody (ADA) to the anti-TIM3 antibody. The secondary outcome measures ORR, mDOR, and PFSR will be measured for up to 12 months of treatment, and Cₘₐₓ, Tₘₐₓ, AUC(0-T), Cₜₐᵤ, AUC(TAU), C_{trough}, Cₑₒᵢ, and ADA will be measured for approximately 2 years.

The clinical study has two arms: Arm A and Arm B. Subjects in Arm A are administered an anti-TIM3 antibody monotherapy at a specified dose on a specified day. Subjects in Arm B are administered an anti-TIM3 antibody and an anti-PD-1 antibody (e.g., nivolumab) combination therapy, wherein the anti-TIM3 antibody is administered at a specific dose on a specific day and the anti-PD-1 antibody (e.g., nivolumab) is administered at a specific dose on a specific day.

Eligible subjects include males and females that are 18 years or older with histologic or cytologic confirmation of a solid tumor that is advanced (metastatic, recurrent, and/or unresectable) with measurable disease, at least one lesion accessible for biopsy, and an Eastern Cooperative Oncology Group Performance Status of 0 or 1. Participants must have received, and then progressed, relapsed, or been intolerant to at least one standard treatment regimen in the advanced or metastatic setting according to solid tumor histologies.

Exclusion criteria include participants with an active, known or suspected autoimmune disease; treatment with a cytotoxic agent, unless at least four weeks have elapsed from the last dose of prior anti-cancer therapy and initiation of the study therapy; and participants with another active malignancy requiring concurrent intervention. Other protocol defined inclusion/exclusion criteria could apply.

This PCT application claims priority benefit of U.S. Provisional Application Nos. 62/617,828, filed Jan. 16, 2018; 62/618,561, filed Jan. 17, 2018; and 62/633,477, filed Feb. 21, 2018, each of which is incorporated herein by reference in its entirety.

What is claimed:

1. A method of treating a subject afflicted with a tumor comprising administering to the subject a therapeutically effective amount of an antibody that binds specifically to a human T-cell immunoglobulin and mucin-domain containing-3 (TIM3) and, e.g., inhibits TIM3 activity (“anti-TIM3 antibody”), wherein the anti-TIM3 antibody is administered at a flat dose ranging from about 4 mg to about 960 mg or a weight-based dose ranging from about 0.05 mg/kg to about 12 mg/kg.

2. The method of claim 1, wherein the anti-TIM3 antibody is administered at a flat dose ranging from about 8 mg to about 800 mg, about 24 mg to about 800 mg, about 72 mg to about 800 mg, about 200 mg to about 800 mg, about 240 mg to about 800 mg, about 300 mg to about 800 mg, about 360 mg to about 800 mg, about 400 mg to about 800 mg, about 480 mg to about 800 mg, 8 mg to about 640 mg, about 24 mg to about 640 mg, about 72 mg to about 640 mg, about 200 mg to about 640 mg, about 240 mg to about 640 mg, about 300 mg to about 640 mg, about 360 mg to about 640 mg, about 400 mg to about 640 mg, about 480 mg to about 640 mg, 8 mg to about 500 mg, about 24 mg to about 500 mg, about 72 mg to about 500 mg, about 200 mg to about 500 mg, about 240 mg to about 500 mg, about 300 mg to about 500 mg, about 360 mg to about 500 mg, about 400 mg to about 500 mg, about 480 mg to about 500 mg, about 240 mg to about 480 mg, or about 360 mg to about 480 mg.

3. The method of claim 1, wherein the anti-TIM3 antibody is administered at a weight-based dose ranging from about 0.1 mg/kg to about 10 mg/kg, about 0.3 mg/kg to about 10 mg/kg, 0.9 mg/kg to about 10 mg/kg, about 1 mg/kg to about 10 mg/kg, about 2.5 mg/kg to about 10 mg/kg, about 3 mg/kg to about 10 mg/kg, about 4 mg/kg to about 10 mg/kg, about 5 mg/kg to about 10 mg/kg, about 6 mg/kg to about 10 mg/kg, about 7 mg/kg to about 10 mg/kg, about 8 mg/kg to about 10 mg/kg, about 9 mg/kg to about 10 mg/kg, about 0.1 mg/kg to about 8 mg/kg, about 0.3 mg/kg to about 8 mg/kg, 0.9 mg/kg to about 8 mg/kg, about 1 mg/kg to about 8 mg/kg, about 2.5 mg/kg to about 8 mg/kg, about 3 mg/kg to about 8 mg/kg, about 4 mg/kg to about 8 mg/kg, about 5 mg/kg to about 8 mg/kg, about 6 mg/kg to about 8 mg/kg, about 7 mg/kg to about 8 mg/kg, about 0.1 mg/kg to about 6 mg/kg, about 0.3 mg/kg to about 6 mg/kg, 0.9 mg/kg to about 6 mg/kg, about 1 mg/kg to about 6 mg/kg, about 2.5 mg/kg to about 6 mg/kg, about 3 mg/kg to about 6 mg/kg, about 4 mg/kg to about 6 mg/kg, or about 5 mg/kg to about 6 mg/kg.

4. The method of claim 1, wherein the anti-TIM3 antibody is administered at a flat dose of about 8 mg, about 24 mg, about 50 mg, about 72 mg, about 100 mg, about 150 mg, about 200 mg, about 240 mg, about 250 mg, about 300 mg, about 350 mg, about 360 mg, about 400 mg, about 450 mg, about 480 mg, about 500 mg, about 540 mg, about 560 mg, about 600 mg, about 640 mg, about 650 mg, about 660 mg, about 700 mg, about 720 mg, about 750 mg, about 760 mg, or about 800 mg.

5. The method of claim 1, wherein the anti-TIM3 antibody is administered at a weight-based dose of about 0.1 mg/kg, about 0.3 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, or about 12 mg/kg.

6. The method of any one of claims 1 to 5, further comprising administering a therapeutically effective amount of an anti-PD-1 antibody.

7. The method of claim 6, wherein the anti-PD-1 antibody is administered at a flat dose ranging from about 80 mg to about 640 mg or a weight-based dose ranging from about 1 mg/kg to about 8 mg/kg.

8. The method of claim 6 or 7, wherein the anti-PD-1 antibody is administered at a flat dose ranging from about 100 mg to about 640 mg, about 120 mg to about 640 mg, about 150 mg to about 640 mg, about 160 mg to about 640 mg, about 180 mg to about 640 mg, about 240 mg to about 640 mg, about 300 mg to about 640 mg, about 320 mg to about 640 mg, about 360 mg to about 640 mg, about 400 mg to about 640 mg, about 420 mg to about 640 mg, about 480 mg to about 640 mg, about 540 mg to about 640 mg, about 100 mg to about 540 mg, about 120 mg to about 540 mg, about 150 mg to about 540 mg, about 160 mg to about 540 mg, about 180 mg to about 540 mg, about 240 mg to about 540 mg, about 300 mg to about 540 mg, about 320 mg to about 540 mg, about 360 mg to about 540 mg, about 400 mg to about 540 mg, about 420 mg to about 540 mg, about 480 mg to about 540 mg, about 100 mg to about 480 mg, about 120 mg to about 480 mg, about 150 mg to about 480 mg, about 160 mg to about 480 mg, about 180 mg to about 480 mg, about 240 mg to about 480 mg, about 300 mg to about 480 mg, about 320 mg to about 480 mg, about 360 mg to about 480 mg, about 400 mg to about 480 mg, about 420 mg to about 480 mg, about 240 mg to about 400 mg, about 300 mg to about 400 mg, about 320 mg to about 400 mg, or about 360 mg to about 400 mg.

9. The method of any one of claims 6 to 8, wherein the anti-PD-1 antibody is administered at a flat dose of about 160 mg, about 200 mg, about 240 mg, about 300 mg, about 360 mg, about 420 mg, about 450 mg, about 480 mg, about 500 mg, about 540 mg, about 600 mg, or about 640 mg.

10. The method of claim 6 or 7, wherein the anti-PD-1 antibody is administered at a weight-based dose ranging from about 1 mg/kg to about 7 mg/kg, about 1 mg/kg to about 6 mg/kg, about 1 mg/kg to about 5 mg/kg, about 1 mg/kg to about 4 mg/kg, about 1 mg/kg to about 3 mg/kg, about 1 mg/kg to about 2 mg/kg, about 2 mg/kg to about 7 mg/kg, about 2 mg/kg to about 6 mg/kg, about 2 mg/kg to about 5 mg/kg, about 2 mg/kg to about 4 mg/kg, about 2 mg/kg to about 3 mg/kg, about 3 mg/kg to about 7 mg/kg, about 3 mg/kg to about 6 mg/kg, about 3 mg/kg to about 5 mg/kg, about 3 mg/kg to about 4 mg/kg, about 4 mg/kg to about 7 mg/kg, about 4 mg/kg to about 6 mg/kg, about 4 mg/kg to about 5 mg/kg, about 5 mg/kg to about 7 mg/kg, about 5 mg/kg to about 6 mg/kg, or about 6 mg/kg to about 7 mg/kg.

11. The method of any one of claims 6, 7, and 10, wherein the anti-PD-1 antibody is administered at a weight-based dose of about 1 mg/kg, about 2 mg/kg, about 3 mg/kg, about 4 mg/kg, about 5 mg/kg, about 6 mg/kg, about 7 mg/kg, or about 8 mg/kg.

12. The method of any one of claims 1 to 11, wherein the anti-TIM3 antibody is administered at a dosing interval of about 1, 2, 3, 4, 5, or 6 weeks.

13. The method of any one of claims 6 to 11, wherein the anti-PD-1 antibody is administered at a dosing interval of about 1, 2, 3, 4, 5, or 6 weeks.

14. The method of any one of claims 6 to 9, wherein the anti-TIM3 antibody is administered at a flat dose of about 200 mg and the anti-PD-1 antibody is administered at a flat dose of about 480 mg.

15. The method of any one of claims 6 to 9, wherein the anti-TIM3 antibody is administered at a flat dose of about 480 mg and the anti-PD-1 antibody is administered at a flat dose of about 480 mg.

16. The method of any one of claims 6 to 9, wherein the anti-TIM3 antibody is administered at a flat dose of about 800 mg and the anti-PD-1 antibody is administered at a flat dose of about 480 mg.

17. The method of any one of claims 14 to 16, wherein the anti-TIM3 antibody is administered at a dosing interval of 4 weeks.

18. The method of any one of claims 14 to 16, wherein the anti-PD-1 antibody is administered at a dosing interval of 4 weeks.

19. The method of any one of claims 6 to 9, wherein the anti-TIM3 antibody is administered at a flat dose of about 4 mg at a dosing interval of 4 weeks and the anti-PD-1 antibody is administered at a flat dose of about 480 mg at a dosing interval of 4 weeks.

20. The method of any one of claims 6 to 9, wherein the anti-TIM3 antibody is administered at a flat dose of about 8 mg at a dosing interval of 4 weeks and the anti-PD-1 antibody is administered at a flat dose of about 480 mg at a dosing interval of 4 weeks.

21. The method of any one of claims 6 to 9, wherein the anti-TIM3 antibody is administered at a flat dose of about 72 mg at a dosing interval of 4 weeks and the anti-PD-1 antibody is administered at a flat dose of about 480 mg at a dosing interval of 4 weeks.

22. The method of any one of claims 6 to 9, wherein the anti-TIM3 antibody is administered at a flat dose of about 150 mg at a dosing interval of 4 weeks and the anti-PD-1 antibody is administered at a flat dose of about 480 mg at a dosing interval of 4 weeks.

23. The method of any one of claims 6 to 9, wherein the anti-TIM3 antibody is administered at a flat dose of about 480 mg at a dosing interval of 4 weeks and the anti-PD-1 antibody is administered at a flat dose of about 480 mg at a dosing interval of 4 weeks.

24. The method of any one of claims 6 to 23, wherein the anti-TIM3 antibody is administered to the subject prior to the administration of the anti-PD-1 antibody.

25. The method of any one of claims 6 to 23, wherein the anti-TIM3 antibody is administered to the subject after the administration of the anti-PD-1 antibody.

26. The method of any one of claims 6 to 23, wherein the anti-TIM3 antibody and the anti-PD-1 antibody are administered concurrently in separate compositions.

27. The method of any one of claims 6 to 23, wherein the anti-TIM3 antibody and the anti-PD-1 antibody are admixed as a single composition for concurrent administration.

28. The method of any one of claims 1 to 27, wherein the tumor is derived from a cancer selected from the group consisting of a bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, renal cancer, lung cancer, stomach cancer, germ cell cancer, bone cancer, liver cancer, thyroid cancer, skin cancer, neoplasm of the central nervous system, lymphoma, leukemia, myeloma, sarcoma, virus-related cancer, and any combinations thereof.

29. The method of claim 28, wherein the cancer is an advanced, recurring, metastatic, and/or refractory cancer.

30. The method of claim 28 or 29, wherein the cancer is a renal cancer (e.g., renal cell carcinoma).

31. The method of claim 28 or 29, wherein the cancer is a colorectal cancer (e.g., colorectal carcinoma).

32. The method of claim 28 or 29, wherein the cancer is a lung cancer (e.g., non-small cell lung cancer).

33. The method of claim 28 or 29, wherein the cancer is a head and neck cancer (e.g., squamous carcinoma of the head and neck).

34. The method of claim 28 or 29, wherein the cancer is a breast cancer (e.g., triple negative breast cancer).

35. The method of claim 28 or 29, wherein the cancer is a skin cancer (e.g., melanoma).

36. The method of claim 28 or 29, wherein the cancer is a bladder cancer (e.g., urothelial carcinoma).

37. The method of claim 28 or 29, wherein the cancer is a lymphoma (e.g., classical Hodgkin's lymphoma).

38. The method of claim 28 or 29, wherein the cancer is a liver cancer (e.g., hepatocellular carcinoma).

39. The method of any one of claims 28 to 38, wherein the cancer is refractory to a prior cancer therapy selected from the group consisting of an anti-angiogenic therapy regimen (e.g., sunitinib, sorafenib, pazopanib, axitinib, tivozanib, and bevacizumab), a standard systemic therapy for metastatic and/or unresectable disease (e.g., Oxaliplatin and Irinotecan), platinum-based chemotherapy, anti-PD(L)-1 therapy, and any combinations thereof.

40. The method of any one of claims 1 to 39, wherein the tumor comprises one or more cells that express human TIM3.

41. The method of any one of claims 1 to 40, wherein the tumor comprises one or more cells that express PD-L1, PD-L2, or both.

42. The method of any one of claims 1 to 41, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

43. The method of any one of claims 1 to 42, wherein the administration reduces the size of the tumor relative to the size of the tumor prior to the administration.

44. The method of claim 43, wherein the size of the tumor is reduced by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as compared to the size of the tumor prior to the administration.

45. The method of any one of claims 1 to 44, wherein the administration induces a proliferation of tumor infiltrating lymphocytes (TILs) in the tumor.

46. The method of any one of claims 6 to 45, wherein the anti-PD-1 antibody cross-competes with nivolumab.

47. The method of any one of claims 6 to 45, wherein the anti-PD-1 antibody is nivolumab.

48. The method of any one of claims 1 to 47, wherein the anti-TIM3 antibody cross-competes for binding to human TIM3 with a reference antibody selected from Table 2.

49. The method of any one of claims 1 to 48, wherein the anti-TIM3 antibody binds to human TIM3 at a same epitope as the reference antibody, as determined by HDX.

50. The method of any one of claims 1 to 49, wherein the anti-TIM3 antibody comprises a heavy chain CDR1, CDR2, and CDR3 and a light chain CDR1, CDR2, and CDR3, wherein
(i) the heavy chain CDR1 comprises an amino acid sequence selected from the group consisting of SEQ ID NO: 41, SEQ ID NO: 42, SEQ ID NO: 43, SEQ ID NO: 44, and SEQ ID NO: 45;
(ii) the heavy chain CDR2 comprises an amino acid sequence selected from the group consisting of SEQ ID NO: 46, SEQ ID NO: 122, SEQ ID NO: 123, SEQ ID NO: 124, SEQ ID NO: 47, SEQ ID NO: 125, SEQ ID NO: 48, SEQ ID NO: 49, SEQ ID NO: 50, SEQ ID NO: 51, SEQ ID NO: 52, SEQ ID NO: 413, and SEQ ID NO: 415;
(iii) the heavy chain CDR3 comprises an amino acid sequence selected from the group consisting of SEQ ID NO: 53, SEQ ID NO: 126, SEQ ID NO: 127, SEQ ID NO: 128, SEQ ID NO: 129, SEQ ID NO: 128, SEQ ID NO: 54, SEQ ID NO: 55, SEQ ID NO: 56, SEQ ID NO: 57, SEQ ID NO: 58, SEQ ID NO: 59, SEQ ID NO: 414, and SEQ ID NO: 416;
(iv) the light chain CDR1 comprises an amino acid sequence selected from the group consisting of SEQ ID NO: 64 and SEQ ID NO: 65;
(v) the light chain CDR2 comprises an amino acid sequence selected from the group consisting of SEQ ID NO: 66 and SEQ ID NO: 67; and/or
(vi) the light chain CDR3 comprises an amino acid sequence selected from the group consisting of SEQ ID NO: 68, SEQ ID NO: 69, SEQ ID NO: 70, SEQ ID NO: 71; and SEQ ID NO: 419.

51. The method of any one of claims 1 to 50, wherein the anti-TIM3 antibody comprises a heavy chain CDR1, CDR2, and CDR3 and a light chain CDR1, CDR2, and CDR3,
(a1) the heavy chain CDR1, CDR2, and CDR3 comprises the amino acid sequences of SEQ ID NOs: 41, 46, and 53, respectively, and the light chain CDR1, CDR2, and CDR3 comprises the amino acid sequences of SEQ ID NOs: 64, 66, and 68, respectively;
(a2) the heavy chain CDR1, CDR2, and CDR3 comprises the amino acid sequences of SEQ ID NOs: 41, 122, and 53, respectively, and the light chain CDR1, CDR2, and CDR3 comprises the amino acid sequences of SEQ ID NOs: 64, 66, and 68, respectively;
(a3) the heavy chain CDR1, CDR2, and CDR3 comprises the amino acid sequences of SEQ ID NOs: 41, 123, and 53, respectively, and the light chain CDR1, CDR2, and CDR3 comprises the amino acid sequences of SEQ ID NOs: 64, 66, and 68, respectively;
(a4) the heavy chain CDR1, CDR2, and CDR3 comprises the amino acid sequences of SEQ ID NOs: 41, 124, and 53, respectively, and the light chain CDR1, CDR2, and CDR3 comprises the amino acid sequences of SEQ ID NOs: 64, 66, and 68, respectively;
(a5) the heavy chain CDR1, CDR2, and CDR3 comprises the amino acid sequences of SEQ ID NOs: 41, 46, and 126, respectively, and the light chain CDR1, CDR2, and CDR3 comprises the amino acid sequences of SEQ ID NOs: 64, 66, and 68, respectively;
(a6) the heavy chain CDR1, CDR2, and CDR3 comprises the amino acid sequences of SEQ ID NOs: 41, 46, and 127, respectively, and the light chain CDR1, CDR2, and CDR3 comprises the amino acid sequences of SEQ ID NOs: 64, 66, and 68, respectively;
(a7) the heavy chain CDR1, CDR2, and CDR3 comprises the amino acid sequences of SEQ ID NOs: 41, 46, and 128, respectively, and the light chain CDR1, CDR2, and CDR3 comprises the amino acid sequences of SEQ ID NOs: 64, 66, and 68, respectively;
(a8) the heavy chain CDR1, CDR2, and CDR3 comprises the amino acid sequences of SEQ ID NOs: 41, 46, and 129, respectively, and the light chain CDR1, CDR2, and CDR3 comprises the amino acid sequences of SEQ ID NOs: 64, 66, and 68, respectively;
(a9) the heavy chain CDR1, CDR2, and CDR3 comprises the amino acid sequences of SEQ ID NOs: 41, 122, and 128, respectively, and the light chain CDR1, CDR2, and CDR3 comprises the amino acid sequences of SEQ ID NOs: 64, 66, and 68, respectively;
(a10) the heavy chain CDR1, CDR2, and CDR3 comprises the amino acid sequences of SEQ ID NOs: 41, 122, and 126, respectively, and the light chain CDR1, CDR2, and CDR3 comprises the amino acid sequences of SEQ ID NOs: 64, 66, and 68, respectively;
(b1) the heavy chain CDR1, CDR2, and CDR3 comprises the amino acid sequences of SEQ ID NOs: 42, 47, and 54, respectively, and the light chain CDR1, CDR2, and CDR3 comprises the amino acid sequences of SEQ ID NOs: 64, 66, and 69, respectively;
(b2) the heavy chain CDR1, CDR2, and CDR3 comprises the amino acid sequences of SEQ ID NOs: 42, 125, and 54, respectively, and the light chain CDR1, CDR2, and CDR3 comprises the amino acid sequences of SEQ ID NOs: 64, 66, and 69, respectively;
(c) the heavy chain CDR1, CDR2, and CDR3 comprises the amino acid sequences of SEQ ID NOs: 43, 48, and 55, respectively, and the light chain CDR1, CDR2, and CDR3 comprises the amino acid sequences of SEQ ID NOs: 64, 66, and 69, respectively;
(d) the heavy chain CDR1, CDR2, and CDR3 comprises the amino acid sequences of SEQ ID NOs: 44, 49, and 56, respectively, and the light chain CDR1, CDR2, and CDR3 comprises the amino acid sequences of SEQ ID NOs: 64, 66, and 68, respectively;
(e1) the heavy chain CDR1, CDR2, and CDR3 comprises the amino acid sequences of SEQ ID NOs: 45, 50, and 57, respectively, and the light chain CDR1, CDR2, and CDR3 comprises the amino acid sequences of SEQ ID NOs: 64, 66, and 69, respectively;
(e2) the heavy chain CDR1, CDR2, and CDR3 comprises the amino acid sequences of SEQ ID NOs: 45, 50, and 57, respectively, and the light chain CDR1, CDR2, and CDR3 comprises the amino acid sequences of SEQ ID NOs: 64, 66, and 71, respectively;
(e3) the heavy chain CDR1, CDR2, and CDR3 comprises the amino acid sequences of SEQ ID NOs: 45, 50, and 57, respectively, and the light chain CDR1, CDR2, and CDR3 comprises the amino acid sequences of SEQ ID NOs: 65, 67, and 70, respectively;
(f) the heavy chain CDR1, CDR2, and CDR3 comprises the amino acid sequences of SEQ ID NOs: 45, 51, and 58, respectively, and the light chain CDR1, CDR2, and CDR3 comprises the amino acid sequences of SEQ ID NOs: 64, 66, and 68, respectively;
(g) the heavy chain CDR1, CDR2, and CDR3 comprises the amino acid sequences of SEQ ID NOs: 45, 52, and 59, respectively, and the light chain CDR1, CDR2, and CDR3 comprises the amino acid sequences of SEQ ID NOs: 64, 66, and 69, respectively;
(h) the heavy chain CDR1, CDR2, and CDR3 comprises the amino acid sequences of SEQ ID NOs: 45, 413, and 414, respectively, and the light chain CDR1, CDR2, and CDR3 comprises the amino acid sequences of SEQ ID NOs: 64, 66, and 69, respectively;
(i1) the heavy chain CDR1, CDR2, and CDR3 comprises the amino acid sequences of SEQ ID NOs: 45, 415, and 416, respectively, and the light chain CDR1, CDR2, and CDR3 comprises the amino acid sequences of SEQ ID NOs: 64, 66, and 68, respectively; or
(i2) the heavy chain CDR1, CDR2, and CDR3 comprises the amino acid sequences of SEQ ID NOs: 45, 415, and 416, respectively, and the light chain CDR1, CDR2, and CDR3 comprises the amino acid sequences of SEQ ID NOs: 64, 66, and 419, respectively.

52. The method of any one of claims 1 to 51, wherein the anti-TIM3 antibody comprises:
(1) a heavy chain variable region comprising an amino acid sequence selected from the group consisting of SEQ ID NO: 34, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 364, SEQ ID NO: 35, SEQ ID NO: 120, SEQ ID NO: 36, SEQ ID NO: 37, SEQ ID NO: 38, SEQ ID NO: 121; SEQ ID NO: 39, SEQ ID NO: 40, SEQ ID NO: 410, SEQ ID NO: 411, and SEQ ID NO: 412; and/or
(2) a light chain variable region comprising an amino acid sequence selected from the group consisting of SEQ ID NO: 60, SEQ ID NO: 61, SEQ ID NO: 62, SEQ ID NO: 63; SEQ ID NO: 417, and SEQ ID NO: 418.

53. The method of any one of claims 1 to 52, wherein the anti-TIM3 antibody is selected from the group consisting an IgG1, an IgG2, an IgG3, an IgG4, and a variant thereof.

54. The method of claim 53, wherein the anti-TIM3 antibody is an IgG1 antibody.

55. The method of claim 54, wherein the anti-TIM3 antibody comprises an effectorless IgG1 Fc.

56. The method of any one of claims 1 to 55, wherein the anti-TIM3 antibody comprises:
(1) a heavy chain comprising an amino acid sequence selected from the group consisting of SEQ ID NO: 15 (or 22), SEQ ID NO: 92 (or 102), SEQ ID NO: 93 (or 103), SEQ ID NO: 94 (or 104), SEQ ID NO: 95 (or 105), SEQ ID NO: 96 (or 106), SEQ ID NO: 97 (or 107), SEQ ID NO: 98 (or 108), SEQ ID NO: 99 or (109), SEQ ID NO: 351 (or 352), SEQ ID NO: 16 (or 23), SEQ ID NO: 100 or (110), SEQ ID NO: 17 (or 24), SEQ ID NO: 18 (or 25), SEQ ID NO: 19 (or 26), SEQ ID NO: 101 (or 111), SEQ ID NO: 20 (or 27), SEQ ID NO: 21 (or 28), SEQ ID NO: 390 (or 391), SEQ ID NO: 398 (or 399), and SEQ ID NO: 404 (or 405); and/or
(2) a light chain comprising an amino acid sequence selected from the group consisting of SEQ ID NO: 29, SEQ ID NO: 30, SEQ ID NO: 33, and SEQ ID NO: 408.

57. The method of any one of claims 1 to 56, wherein the anti-TIM3 antibody comprises a heavy chain and a light chain, wherein:
(a1) the heavy chain comprises the amino acid sequence of SEQ ID NO: 15 (or 22) and the light chain comprises the amino acid sequence of SEQ ID NO: 29;
(a2) the heavy chain comprises the amino acid sequence of SEQ ID NO: 92 (or 102) and the light chain comprises the amino acid sequence of SEQ ID NO: 29;
(a3) the heavy chain comprises the amino acid sequence of SEQ ID NO: 93 (or 103) and the light chain comprises the amino acid sequence of SEQ ID NO: 29;
(a4) the heavy chain comprises the amino acid sequence of SEQ ID NO: 94 (or 104) and the light chain comprises the amino acid sequence of SEQ ID NO: 29;
(a5) the heavy chain comprises the amino acid sequence of SEQ ID NO: 95 (or 105) and the light chain comprises the amino acid sequence of SEQ ID NO: 29;
(a6) the heavy chain comprises the amino acid sequence of SEQ ID NO: 96 (or 106) and the light chain comprises the amino acid sequence of SEQ ID NO: 29;
(a7) the heavy chain comprises the amino acid sequence of SEQ ID NO: 97 (or 107) and the light chain comprises the amino acid sequence of SEQ ID NO: 29;
(a8) the heavy chain comprises the amino acid sequence of SEQ ID NO: 98 (or 108) and the light chain comprises the amino acid sequence of SEQ ID NO: 29;
(a9) the heavy chain comprises the amino acid sequence of SEQ ID NO: 99 or (109) and the light chain comprises the amino acid sequence of SEQ ID NO: 29;
(a10) the heavy chain comprises the amino acid sequence of SEQ ID NO: 351 (or 352) and the light chain comprises the amino acid sequence of SEQ ID NO: 29;
(b1) the heavy chain comprises the amino acid sequence of SEQ ID NO: 16 (or 23) and the light chain comprises the amino acid sequence of SEQ ID NO: 30;
(b2) the heavy chain comprises the amino acid sequence of SEQ ID NO: 100 or (110) and the light chain comprises the amino acid sequence of SEQ ID NO: 30;
(c) the heavy chain comprises the amino acid sequence of SEQ ID NO: 17 (or 24) and the light chain comprises the amino acid sequence of SEQ ID NO: 30;
(d) the heavy chain comprises the amino acid sequence of SEQ ID NO: 18 (or 25) and the light chain comprises the amino acid sequence of SEQ ID NO: 29;
(e1) the heavy chain comprises the amino acid sequence of SEQ ID NO: 19 (or 26) and the light chain comprises the amino acid sequence of SEQ ID NO: 33;
(e2) the heavy chain comprises the amino acid sequence of SEQ ID NO: 101 (or 111) and the light chain comprises the amino acid sequence of SEQ ID NO: 33;
(f) the heavy chain comprises the amino acid sequence of SEQ ID NO: 20 (or 27) and the light chain comprises the amino acid sequence of SEQ ID NO: 29;
(g) the heavy chain comprises the amino acid sequence of SEQ ID NO: 21 (or 28) and the light chain comprises the amino acid sequence of SEQ ID NO: 30;
(h) the heavy chain comprises the amino acid sequence of SEQ ID NO: 390 (or 391) and the light chain comprises the amino acid sequence of SEQ ID NO: 408;
(i1) the heavy chain comprises the amino acid sequence of SEQ ID NO: 398 (or 399) and the light chain comprises the amino acid sequence of SEQ ID NO: 29; or
(i2) the heavy chain comprises the amino acid sequence of SEQ ID NO: 404 (or 405) and the light chain comprises the amino acid sequence of SEQ ID NO: 29.

58. The method of any one of claims 1 to 57, wherein the administration is performed in combination with an additional therapeutic agent.

59. The method of claim 58, wherein the additional therapeutic agent is selected from the group consisting of a chemotherapy, radiation, surgery, hormone deprivation, angiogenesis inhibitors, additional immune checkpoint inhibitors, and any combinations thereof.

60. The method of claim 59, wherein the additional immune checkpoint inhibitors comprise an anti-LAG-3 antibody, an anti-CTLA-4 antibody, an anti-GITR antibody, or an anti-PD-L1 antibody.

61. The method of any one of claims 1 to 60, wherein the subject has received, and then progressed, relapsed, or been intolerant to at least one standard treatment regimen, e.g., in the advanced or metastatic setting according to solid tumor histologies.

62. The method of any one of claims 1 to 61, wherein the tumor comprises a solid tumor.

63. The method of any one of claims 1 to 62, wherein the tumor comprises a solid tumor that is advanced.

64. The method of any one of claims 1 to 63, wherein the tumor comprises a solid tumor that has spread.

65. The method of any one of claims 1 to 64, wherein the tumor comprises an advanced malignant tumor.
